(19) United States
(12) Patent Application Publication
(10) Pub. No.: US 2010/0069441 A1
(43) Pub. Date: Mar. 18, 2010

(54) ANTIMICROBIAL INDOLINE COMPOUNDS FOR TREATMENT OF BACTERIAL INFECTIONS

(76) Inventors: Mikhail Fedorovich Gordeev, Castro Valley, CA (US); Zhengyu Yuan, Palo Alto, CA (US); Jinqian Liu, Palo Alto, CA (US); Qiang Wang, Union City, CA (US)

Correspondence Address: JONES DAY, 222 EAST 41ST ST, NEW YORK, NY, 10017, US

(21) Appl. No.: 12/552,245

(22) Filed: Sep. 1, 2009

Related U.S. Application Data
(60) Provisional application No. 61/093,696, filed on Sep. 2, 2008.

(51) Int. Cl. A61K 31/4439; C07D 413/10; A61P 31/04; A61P 31/12

(52) U.S. Cl. 514/338; 546/271.7

(57) ABSTRACT

The present invention provides indoline heterocyclic compounds of the following formula I:

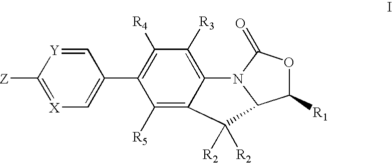

or pharmaceutically acceptable salts, prodrugs, solvates, or hydrates thereof useful as antibacterial agents, pharmaceutical compositions containing them, methods for their use, and methods for preparing these compounds.

### RELATED APPLICATIONS

This application claims the benefit under 35 U.S.C. §119 of U.S. Provisional Application No. 61/093,696, filed Sep. 2, 2008, incorporated herein by reference in its entirety.

### FIELD OF THE INVENTION

The present invention provides novel indoline heterocyclic compounds with useful antimicrobial properties, pharmaceutical compositions thereof, methods for their use, and methods for preparing of the same. These compounds have potent activities against pathogenic bacterial species.

### BACKGROUND OF THE INVENTION

Due to an increasing antibiotic resistance, novel classes of antibacterial compounds are acutely needed for the treatment of bacterial infections. The antibacterials should possess useful levels of activity against certain human and veterinary pathogens, including gram-positive aerobic bacteria such as multiply-resistant staphylococci and streptococci, select anaerobes such as bacteroides and clostridia species, and acid-fast microorganisms such as Mycobacterium tuberculosis and Mycobacterium avium.

It is also important that such antibacterial agents should offer sufficient safety with a minimal toxicity and adverse effects that can preclude or limit the therapy.

Among newer antibacterial agents, heterocyclic oxazolidinone compounds are the most recent synthetic class of antimicrobials that are active against several key pathogenic microbes, including methicillin-resistant Staphylococcus aureus (MRSA). To date, a sole antibacterial of this class linezolid (Zyvox^{R}) has been approved for a treatment of select gram-positive infections. While the drug is widely used in the antimicrobial therapy, its indications are limited, in part, due to a modest activity against fastidious gram-negative pathogens, such as Haemophilus influenzae. Furthermore, an emergence of linezolid-resistant bacteria such as linezolid-resistant Enterococcus faecium and Staphylococcus aureus strains, have been described, for example, in The Lancet, 2001, p. 207. Typical for all antimicrobial agents, the resistance is expected to become more problematic with a continued linezolid use. Thus, newer agents with an improved activity and bacterial spectrum are needed.

The utility of oxazolidinone antibacterials also can be restricted due to serious adverse effects. Among these, monoamine oxidase inhibition and myelosuppression or bone marrow toxicity are among key factors limiting linezolid utility, as reflected in warnings in the prescribing information for linezolid (Zyvox^{R}) The bone marrow suppression (also referred to as hematopoietic toxicity or myelosuppression) was reported, for example, by Monson et al. in Clinical Infectious Diseases, 2002, vol. 35, pp. e29-31. Several adverse effects for linezolid (Zyvox^{R}) (including anemia, leukopenia, pancytopenia, and thrombocytopenia) have been ascribed to this phenomenon.

None of aforementioned publications specifically contemplates compounds of the present invention, their beneficial safety profiles, their combination therapies, or their novel compositions.

### SUMMARY OF THE INVENTION

The present invention provides novel indoline oxazolidinone compounds with useful antibacterial activity. The activity for compounds of this invention includes antibacterial activity against gram-positive microorganisms, such as Staphylococcus aureus, Streptococcus pneumoniae, Enterococcus faecalis, and Enterococcus faecium, as well as some linezolid-resistant and fastidious gram-negative strains. Within the scope of the invention, heterocyclic structures comprising an oxazolidinone ring fused to a substituted indoline fragment (featuring at least one unique heterocyclic substituent) can provide therapeutically useful compounds with high antimicrobial activity

Surprisingly, certain compounds of the present invention are active against select multi-drug resistant bacteria, including MRSA, and against linezolid-resistant gram-positive bacteria, such as linezolid-resistant Enterococcus faecium or Staphylococcus aureus. Furthermore, certain compounds of the present invention are also active against fastidious gram-negative pathogens, such as Haemophilus influenzae. The compounds provided herein can be useful as antibacterial agents for treatment of infections including, but not limited to, skin infections, soft tissue infections, bacteremia, respiratory tract infections, urinary tract infections, bone infections, and eye infections.

In addition, certain compounds of this invention also offer a beneficially reduced propensity for monoamine oxidase and/or myelosuppression.

The present invention provides a compound of the following formula A:

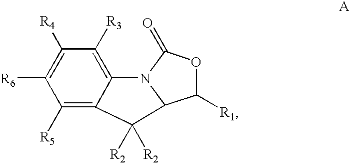

or a pharmaceutically acceptable salt, prodrug, solvate, or hydrate thereof wherein:
R¹ is CH₂OH, CH₂NHC(═O)C₁₋₅ alkyl, CH₂NHC(═O)OC₁₋₅alkyl, CH₂NH-Het¹, CH₂O-Het¹, CH₂Het¹, CH₂Het², CH₂OPO₃H₂, CH₂OC(═O)CH₂(CH₂)ₘOPO₃H₂, or CH₂OC(═O)CH(NH₂)C₁₋₄alkyl, wherein m is 1, 2, or 3
each R² is independently H or F;
R³, R⁴, and R⁵ are independently H, F, Cl, CN, CH₃, or OH; and
R⁶ is substituted or unsubstituted aryl, biaryl, Het¹, Het², or 4 to 7 membered heterocyclic group.

The alkyl, alkenyl, or cycloalkyl groups at each occurrence above independently can be optionally substituted with one, two, or three substituents each independently selected from the group consisting of halo, aryl, Het¹, and Het². Het¹ at each occurrence can be independently a C-linked 5 or 6 membered heterocyclic ring having 1 to 4 heteroatoms each independently selected from the group consisting of oxygen, nitrogen, and sulfur within the ring. Het² at each occurrence can be independently a N-linked 5 or 6 membered heterocyclic ring having 1 to 4 nitrogen and optionally having one oxygen or sulfur within the ring.

In certain aspects, R¹ in a compound of formula A is CH₂OH, CH₂OPO₃H₂, CH₂OC(═O)CH₂CH₂CH₂OPO₃H₂, or CH₂OC(═O)CH(NH₂)C₁₋₄alkyl.

In certain aspects, R¹ in a compound of formula A is CH₂OH.

In certain aspects, R¹ in a compound of formula A is CH₂NHC(═O)OC₁₋₃alkyl or CH₂NHC(═O)Het¹.

In certain aspects, R¹ in a compound of formula A is (4-R⁷-1,2,3-triazol-1-yl)methyl, wherein R⁷ is H, C₁₋₃alkyl, halo, or CN.

In certain aspects, R¹ in a compound of formula A is (5-R⁷-isoxazol-3-yl)aminomethyl or (5-R⁷-isoxazol-3-yl)oxymethyl, wherein R⁷ is H, C₁₋₅alkyl, halo, or CN.

In certain aspects, R¹ in a compound of formula A is (4-R⁷-1,2,3-triazol-1-yl)methyl, (5-R⁷-isoxazol-3-yl)aminomethyl or (5-R⁷-isoxazol-3-yl)oxymethyl, wherein R⁷ is H, C₁₋₃alkyl, halo, or CN.

In certain aspects, R² in a compound of formula A is H.

In certain aspects, R³, R⁴, and R⁵ in a compound of formula A are each independently selected from H and F.

In certain aspects, a compound of formula A has a stereochemistry represented by the following formula B:

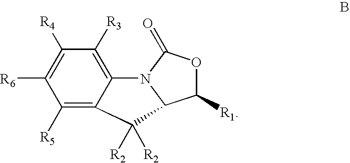

In certain aspects of formula B, R⁶ is 4-Het¹-phenyl, 4-Het²-phenyl, 2-Het¹-pyridin-5-yl, 2-Het²-pyridin-5-yl, 2-Het¹-pyrimidin-5-yl, or 2-Het²-pyrimidin-5-yl.

In certain aspects of formula B, R⁶ is 4-(Het¹-CH₂—W—CH₂)-phenyl, 4-(Het²-CH₂—W—CH₂)-phenyl, 2-(Het¹-CH₂—W—CH₂)-pyridine-5-yl, or 2-(Het²-CH₂—W—CH₂)-pyridin-5-yl; wherein W is NH, NC₁₋₅alkyl, NC(═O)OC₁₋₅alkyl, NC(═O)C₁₋₅alkyl, NC(═O)Het¹, O, S, S(O)ₙ, and wherein n is 0, 1, or 2.

In another aspect, compounds of formula A are selected from formula I

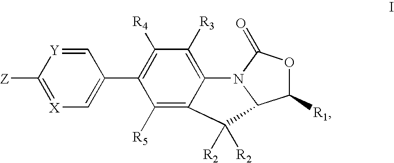

wherein:

R¹ is CH₂OH, CH₂NHC(═O)C₁₋₅alkyl, CH₂NHC(═O)OC₁₋₅alkyl, CH₂NH-Het¹CH₂O-Het¹CH₂Hetⁱ, CH₂Het², CH₂OPO₃H₂, CH₂OC(═O)CH₂CH₂CH₂OPO₃H₂, or CH₂OC(═O)CH(NH₂)C₁₋₄alkyl;

R² is H or F;

R³, R⁴, and R⁵ are each independently H, F, Cl, CN, CH₃, or OH;

X and Y are each independently CH, CF, N, or N⁺—O⁻; and

Z is Het¹, Het², 4 to 7-membered heterocyclic group, CN, CONH₂, CONHC₁₋₆alkyl, NH—C(═O)H, NH—C(═O)C₁₋₆alkyl, NH—SO₂C₁₋₆alkyl, NH—C(═O)OC₁₋₆alkyl, NHC(═O)NHC₁₋₆alkyl, Het¹-CH₂—W—CH₂ or Het²-CH₂—W—CH₂—, wherein W is CH₂, NH, NC₁₋₅alkyl, NC(═O)OC₁₋₅alkyl, NC(═O)C₁₋₅alkyl, NC(═O)Het¹O, S, or S(O), and wherein n is 0, 1, or 2.

In certain embodiments, R¹ is CH₂OH, CH₂NHC(═O)OC₁₋₅alkyl, CH₂NH-Het¹CH₂O-Het¹CH₂Het¹, CH₂Het², CH₂OPO₃H₂, CH₂OC(═O)CH₂CH₂CH₂OPO₃H₂, or CH₂OC(═O)CH(NH₂)C₁₋₄alkyl.

In certain aspects, compounds of formula I are selected with a proviso that when R¹ is CH₂NHCOR′,

wherein R′ is selected from
H;
C₁₋₁₂alkyl,
C₁₋₁₂alkyl optionally substituted with 1-3 Cl;
CH₂OH;
CH₂OC₁₋₁₂alkyl;
C₃₋₁₂cycloalkyl;
phenyl optionally substituted with 1-3 of groups OH, OMe, OEt, NO₂, halo, COOH, SO₃H, or NR″R′″, wherein R″ and R′″ are selected from H and C₁₋₁₂alkyl;
furanyl;
tetrahydrofuranyl;
2-thiophene;
pyrrolidinyl;
pyridinyl;
OC₁₋₁₂alkyl;
NH₂;
NHC₁₋₂alkyl;
NHPh;
COPh; and
   X is CH or CF, and Y is N or N⁺—O⁻; then
   Z is other than H, C₁₋₄alkyl, NO₂, NH₂, NHC(═O)C₁₋₄ alkyl, CN, COOH, OC₁₋₄alkyl, or halo.

In certain aspects, X in compounds of formula I is CH or CF, and Y in compounds of formula I is N or N⁺—O⁻, with a proviso that

when R¹ is CH₂NHCOR′, wherein R′ is selected from
H;
C₁₋₁₂alkyl,
C₁₋₁₂alkyl optionally substituted with 1-3 Cl;
CH₂OH;
CH₂OC₁₋₁₂alkyl;
C₃₋₁₂cycloalkyl;
phenyl optionally substituted with 1-3 of groups OH, OMe, OEt, NO₂, halo, COOH, SO₃H, or NR″R′″, wherein R″ and R′″ are selected from H and C₁₋₁₂alkyl;
furanyl;
tetrahydrofuranyl;
2-thiophene;
pyrrolidinyl;
pyridinyl;
OC₁₋₁₂alkyl;
NH₂;
NHC₁₋₁₂alkyl;
NHPh; and
COPh; then
   Z is other than H, C₁₋₄alkyl, NO₂, NH₂, NHC(═O)C₁₋₄alkyl, CN, COOH, OC₁₋₄alkyl, or halo.

In certain embodiments, Z is Het¹, Het², 4 to 7-membered heterocyclic group, CONH₂, CONHC₁₋₆alkyl, NH—C(═O)H, NH—SO₂C₁₋₆alkyl, NH—C(═O)OC₁₋₆alkyl, NHC(═O)NHC₁₋₆alkyl, Het¹-CH₂—W—CH₂, or Het²-CH₂—W—CH₂—; wherein W is CH₂, NH, NC₁₋₅alkyl, NC(═O)OC₁₋₅alkyl, NC(═O)C₁₋₅alkyl, NC(═O)Het¹, O, S, or S(O), and wherein n is 0, 1, or 2.

In certain embodiments, the compound provided herein is not N-(((1S,9aS)-7-(6-cyanopyridin-3-yl)-3-oxo-1,3,9,9a-tetrahydrooxazolo[3,4-a]indol-1-yl)methyl)acetamide. In certain embodiments, the compound provided herein is not N-(((1S,9aS)-7-(4-cyanophenyl)-3-oxo-1,3,9,9a-tetrahydrooxazolo[3,4-a]indol-1-yl)methyl)acetamide.

In certain aspects, Z in compounds of formula I is selected from Het¹ or Het².

In certain other aspects, Z in compounds of formula I is selected from Het¹-CH₂—W—CH₂—, and Het²-CH₂—W—CH₂—, wherein W is CH₂, NH, NC₁₋₅alkyl, NC(═O)OC₁₋₅alkyl, NC(═O)C₁₋₅alkyl, NC(═O)Het¹, O, S, S(O), and wherein n is 0, 1, or 2.

In another aspect, compounds of formula I are selected from formula II

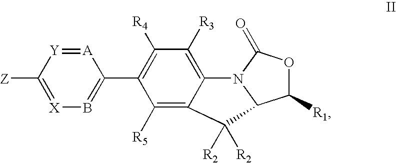

wherein A, B, X, and Y are independently CH, CF, or N.

In another aspect, the present invention provides a pharmaceutical composition comprising a compound of any of formulas A, B, I, or II, or a pharmaceutically acceptable salt thereof, and a pharmaceutically acceptable carrier.

In another aspect, the present invention provides a method for treating microbial infection in a mammal by administering to the mammal in need a therapeutically effective amount of a compound of any of formulas A, B, I, or II, or a pharmaceutically acceptable salt thereof.

In certain aspects, the microbial infection is a gram-positive microbial infection.

In certain aspects, the microbial infection is a gram-positive microbial infection caused by linezolid-resistant bacteria.

In certain aspects, the microbial infection is a fastidious gram-negative microbial infection.

The compounds of formulas A, B, I, or II may be administered orally, parenterally, transdermally, topically, rectally, or intranasally.

The compounds of formulas A, B, I, or II may be administered once-daily in an amount of from about 1 to about 75 mg/kg of body weight/day.

In certain aspects, provided herein is a compound according to any one of formulas A, B, I, or II for use in therapy.

In certain aspects, provided herein is a compound according to any one of formulas A, B, I, or II for use in the treatment of a microbial infection in a mammal in need thereof.

In certain aspects, provided herein is use of a compound according to any one of formulas A, B, I, or II in the manufacture of a medicament for therapy.

In certain aspects, provided herein is use of a compound according to any one of formulas A, B, I, or II in the manufacture of a medicament for treatment of a bacterial infection in a mammal in need thereof. In another aspect, the compounds of formulas A, B, I, or II can be used in combinations with other bioactive agents, such as anti-infective or anti-inflammatory agents. For example, to achieve an optimal therapeutic effect (such as a broad spectrum of action), compounds of formulas A, B, I, or II may be co-administered in a combination with an antimicrobial agent active against gram-negative bacteria (e.g., quinolone, beta-lactam, aminoglycoside, colistin, macrolide agent, etc.), an agent active against pathogenic fungi or yeast (e.g., allylamine, terbinafine, azole, etc.), or in combination with an antiviral agent (such as an entry-blocker, viral protease or DNA inhibitor, antiretroviral agent, etc.).

In yet another aspect, the present invention provides novel intermediates and processes for preparing compounds of formulas A, B, I, and II.

### DETAILED DESCRIPTION OF THE INVENTION

Unless otherwise stated, the following terms used in the specification and Claims have the meanings given below:

The carbon atom content of various hydrocarbon-containing moieties is indicated by a prefix designating the minimum and maximum number of carbon atoms in the moiety, i.e., the prefix Cᵢ₋ⱼ indicates a moiety of the integer to the integer “j” carbon atoms, inclusive. Thus, for example, C₁₋₇ alkyl refers to alkyl of one to seven carbon atoms, inclusive.

Group R^{#} is same as R_{#}: R¹ is same as R₁, etc.

The terms “alkyl,” “alkenyl,” etc. refer to both straight and branched groups, but reference to an individual radical such as “propyl” embraces only the straight chain radical, a branched chain isomer such as “isopropyl” being specifically referred to. The alkyl, alkenyl, etc., group may be optionally substituted with one, two, or three substituents selected from the group consisting of halo, CN, OH, NH₂, aryl, Het¹, or Het².

The term “cycloalkyl” means a cyclic saturated monovalent hydrocarbon group of three to six carbon atoms, e.g., cyclopropyl, cyclohexyl, and the like. The cycloalkyl group may be optionally substituted with one, two, or three substituents selected from the group consisting of halo, CN, OH, NH₂, aryl, Het¹, or Het².

The term “heteroalkyl” means an alkyl or cycloalkyl group, as defined above, having a substituent containing a heteroatom selected from N, O, or S(O), where n is an integer from 0 to 2, including, hydroxy (OH), C₁₋₄alkoxy, amino, thio (—SH), and the like. Representative substituents include —NRₐR_{b}, —ORₐ, or —S(O)ₙR_{c}, wherein Rₐ is hydrogen, C₁₋₄alkyl, C₃₋₆cycloalkyl, optionally substituted aryl, optionally substituted heterocyclic, or —COR (where R is C₁₋₄alkyl); R_{b} is hydrogen, C₁₋₄alkyl, —SO₂R (where R is Cᵢ alkyl or C₁₋₄hydroxyalkyl), —SO₂NRR′ (where R and R′ are independently of each other hydrogen or C₁₋₄alkyl), —CONR′R″ (where R′ and R″ are independently of each other hydrogen or C₁₋₄alkyl); n is an integer from 0 to 2; and R_{c} is hydrogen, C₁₋₄alkyl, C₃₋₆cycloalkyl, optionally substituted aryl, or NRₐR_{b} where Rₐ and R_{b} are as defined above. Representative examples include, but are not limited to, 2-methoxyethyl (—CH₂CH₂OCH₃), 2-hydroxyethyl (—CH₂CH₂OH), hydroxymethyl (—CH₂OH), 2-aminoethyl (—CH₂CH₂NH₂), 2-dimethylaminoethyl (—CH₂CH₂NHCH₃), benzyloxymethyl, thiophen-2-ylthiomethyl, and the like.

The term “halo” refers to fluoro (F), chloro (Cl), bromo (Br), or iodo (I).

The term “aryl” refers to phenyl, biphenyl, or naphthyl, optionally substituted with 1 to 3 substituents independently selected from halo, —C₁₋₄alkyl, —OH, —OC₁₋₄alkyl, —S(O)_{N}C₁₋₄alkyl wherein n is 0, 1, or 2, —C₁₋₄alkylNH₂, —NHC₁₋₄alkyl, —C(═O)H, or —C═N—OR_{D} wherein R_{d} is hydrogen or —C₁₋₄alkyl. Likewise, the term phenyl refers to the phenyl group optionally substituted as above.

The term “heterocyclic ring” refers to an aromatic ring or a saturated or unsaturated ring that is not aromatic of 3 to 10 carbon atoms and 1 to 4 heteroatoms selected from the group consisting of oxygen, nitrogen, and S(O)ₙ within the ring, where n is defined above. The heterocyclic ring may be optionally substituted with halo, —C₁₋₄alkyl, —OH, —OC₁₋₄ alkyl, —S(O)ₙC₁₋₄alkyl wherein n is 0, 1, or 2, —C₁₋₄alkylNH₂, —NHC₁₋₄alkyl, —C(═O)H, or —C═N—OR_{d} wherein R_{d} is hydrogen or C₁₋₄alkyl.

Examples of heterocylic rings include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isoxazolinone, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiadiazole tetrazole, thiazolidine, thiophene, benzo[b]thiophene, morpholinyl, thiomorpholinyl (also referred to as thiamorpholinyl), piperidinyl, pyrrolidine, tetrahydrofuranyl, 1,3-benzoxazine, 1,4-oxazine-3-one, 1,3-benzoxazine-4-one, pyrrolidine, pyrrolidine-2-one, oxazolidine-2-one, azepine, perhydroazepine, perhydroazepine-2-one, perhydro-1,4-oxazepine, perhydro-1,4-oxazepine-2-one, perhydro-1,4-oxazepine-3-one, perhydro-1,3-oxazepine-2-one and the like. Heterocyclic rings include unsubstituted and substituted rings.

Specifically, Het¹ (same as het¹, Het₁ or hetᵢ) refers to a C-linked five- (5) or six- (6) membered heterocyclic ring, including bicyclic rings. Each Het¹ may be optionally substituted with 1 to 3 substituents independently selected from alkyl, haloalkyl, hydroxyalkyl, aminoalkyl, or cyanoalkyl, halo, CN, or NH₂. Representative examples of substituents include, but are not limited to, fluoromethyl, difluoromethyl, 2-fluoroethyl, trifluoroethyl, hydroxymethyl, 2-hydroxypropyl, aminomethyl, and cyanomethyl.

Representative examples of “Het¹” include, but are not limited to, pyridine, thiophene, furan, pyrazole, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, 1,2,4-dithiazolone, 3-azabicyclo[3.1.0]hexan-6-yl, 2-C₁₋₄alkyl-tetrazole, 1-C₁₋₁₄alkyl-tetrazole, 1,2,3-triazole, 1-C₁₋₄alkyl-triazole, 2-C₁₋₄-alkyl-triazole, 4-C₁₋₄alkyl-1,2,3-triazole, 5-C₁₋₄alkyl-1,2,3-triazole, 1,2,4-triazole, 2-C₁₋₄alkyl-1,2,4-triazole, 3-C₁₋₄alkyl-1,2,4-triazole, 1-C₁₋₄alkyl-1,2,4-triazole, 3-C₁₋₄alkyl-1,2,4-triazole, 4-C₁₋₄alkyl-1,2,4-triazole, 5-C₁₋₄alkyl-1,2,4-triazole, 1,3,4-oxadiazole, 2-C₁₋₄alkyl-1,3,4-oxadiazole, 5-C₁₋₄alkyl-1,3,4-oxadiazole, 1,3,4-thiadiazole, 2-C₁₋₄alkyl-1,3,4-thiadiazole, 5-C₁₋₄alkyl-1,3,4-thiadiazole, 1,2,4-oxadiazole, 3-C₁₋₄alkyl-1,2,4-oxadiazole, 5-C₁₋₄alkyl-1,2,4-oxadiazole, oxazole, 2-C₁₋₄alkyl-oxazole, 4-C₁₋₄alkyl-oxazole, 5-C₁₋₄alkyl-oxazole, thiazole, 2-C₁₋₄-alkyl-thiazole, 4-C₁₋₄alkyl-thiazole, 5-C₁₋₄alkyl-thiazole, isoxazole, 3-C₁₋₄alkyl-isoxazole, 4-C₁₋₄alkyl-isoxazole, 5-C₁₋₄alkyl-isoxazole, isoxazoline, 3-C₁₋₄alkyl-isoxazoline, 4-C₁₋₄alkyl-isoxazoline, 5-C₁₋₄alkyl-isoxazoline, isothiazole, 3-C₁₋₄alkyl-isothiazole, 4-C₁₋₄alkyl-isothiazole, or 5-C₁₋₄alkyl-isothiazole, pyrazole, 1-C₁₋₄alkylpyrazole, 3-C₁₋₄alkyl pyrazole, 4-C₁₋₄alkylpyrazole, 5-C₁₋₄alkylpyrazole. For example, Het¹ can be independently a carbon-connected tetrazole, 1,2,3-triazole, 1,2,4-triazole, 1,3,4-oxadiazole, 1,3,4-thiadiazole, 1,2,4-oxadiazole, oxazole, thiazole, isoxazole, isothiazole, isoxazoline, or pyrazole group.

Het² (same as het², Het₂, or het₂) refers to an N-linked five- (5) or six- (6) membered heterocyclic ring having 1 to 4 nitrogen atoms, and optionally having one oxygen or sulfur atom, including bicyclic rings. Each Het² may be optionally substituted with 1 to 3 substituents independently selected from alkyl, haloalkyl, hydroxyalkyl, aminoalkyl, cyanoalkyl, halo, CN, or NH₂. Representative examples of substituents include, but are not limited to, fluoromethyl, difluoromethyl, 2-fluoroethyl, trifluoroethyl, hydroxymethyl, 2-hydroxypropyl, aminomethyl, and cyanomethyl.

Representative examples of “Het²” include, but are not limited to pyrrolyl, imidazolyl, pyrazolyl, 1,2,3-triazolyl, 1,2,4-triazolyl, 1,2,3,4-tetrazolyl, isoxazolidinonyl group, 3-azabicyclo[3.1.0]hexan-3-yl, 1,3,9,9a-tetrahydrooxazolo[3,4-a]indol-1-yl, 2-alkylpyrrolo[3,4-c]pyrazol-5(2H,4H,6H)-yl, and 5H-pyrrolo[3,4-b]pyridin-6(7H)-yl, tetrazole, 5-C₁₋₄alkyl-tetrazole, 1,2,3-triazole, 4-C₁₋₄alkyl-1,2,3-triazole, 5-C₁₋₄alkyl-1,2,3-triazole, 4-C₁₋₄alkyl-5-C₁₋₄alkyl-1,2,3-triazole 1,2,4-triazole, 3-C₁₋₄alkyl-1,2,4-triazole, 5-C₁₋₄alkyl-1,2,4-triazole, oxazolidinone, 4-C₁₋₄alkyl-oxazolidinone, 5-C₁₋₄alkyl-oxazolidinone, pyrrolidin-2-one, 3-C₁₋₄alkyl-pyrrolidin-2-one, 4-C₁₋₄alkyl-pyrrolidin-2-one, 5-C₁₋₄alkyl-pyrrolidin-2-one, imidazolidin-2-one, 3-C₁₋₄alkyl-imidazolidin-2-one, 4-5-C₁₋₄alkyl-imidazolidin-2-one, imidazole, 2-C₁₋₄alkyl-imidazole, 4-C₁₋₄alkyl-imidazole, 5-C₁₋₄alkyl-imidazole, pyrazole, 3-C₁₋₄alkyl-pyrazole, 4-C₁₋₄alkyl-pyrazole, 5-C₁₋₄-alkyl-pyrazole, 2-C₁₋₄alkyl-3-C₁₋₄-alkyl-imidazole. For example, Het² can be independently a nitrogen-connected tetrazole, 1,2,3-triazole, 1,2,4-triazole, oxazolidinone, pyrrolidin-2-one, imidazolidin-2-one, pyrazole, or imidazole group.

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

Compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and Claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A hydrogen (H) or carbon (C) substitution for compounds of the formula I include a substitution with any isotope of the respective atom. Thus, a hydrogen (H) substitution includes a ¹H, ²H (deuterium), or ³H (tritium) isotope substitution, as may be desired, for example, for a specific therapeutic, diagnostic therapy, or metabolic study application. Optionally, a compound of this invention may incorporate a known in the art radioactive isotope or radioisotope, such as ³H, ¹⁵O, ¹⁴C, or ¹³N isotope, to afford a respective radiolabeled compound of formula I.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It is understood that a specific salt form can be advantageous compared to an acidic form of a compound of Claim 1. For example, a less acidic sodium salt of a phosphoric acid compound can be advantageous for an appropriate therapeutic application (e.g., intravenous injection or infusion). Furthermore, a specific salt form could be advantageous to maximize compound shelf life and stability. Thus, a mono-sodium or mono-amine (i.e. mono-ammonium) salt of a phosphoric acid compound in certain instances may be more stable than a di-sodium or di-amine salt of said phosphoric acid compound.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

“Infection” means any microbial infection, such as gram-positive, gram-negative, or fungal infection.

A “therapeutically effective amount” means the amount of a compound that, when administered to a mammal for treating a disease, is sufficient to effect such treatment for the disease. The therapeutically effective amount will vary depending on the compound, the disease and its severity and the age, weight, etc., of the mammal to be treated. Therapeutically effective amount may also be referred to as any amount of a compound that is sufficient to achieve the desired beneficial effect, including preventing the disease, inhibiting the disease, or relieving the disease, as described above in (1)-(3). For example, the amount of a compound can range between 0.1-250 mg/kg, or preferably, 0.5-100 mg/kg, or more preferably, 1-50 mg/kg, or even more preferably, 2-20 mg/kg. More preferably, said amount of a compound is administered to a mammal once-daily. Even more preferably, said amount of a compound is administered to a mammal once-weekly or once-biweekly.

“Leaving group” has the meaning conventionally associated with it in synthetic organic chemistry, i.e., an atom or group capable of being displaced by a nucleophile and includes halogen, C₁₋₄alkylsulfonyloxy, ester, or amino such as chloro, bromo, iodo, mesyloxy, tosyloxy, trifluorosulfonyloxy, methoxy, N,O-dimethylhydroxyl-amino, and the like.

“Prodrug” means any compound which releases an active parent drug according to a compound of the subject invention in vivo when such prodrug is administered to a mammalian subject. Various prodrugs have been described, for example, in the following publications: Alexander et al. J. Med. Chem. 1988, p. 318; Alexander et al. J. Med. Chem., 1991, p. 78; Murdock et al. J. Med. Chem., 1993, p. 2098; Davidsen et al. J. Med. Chem., 1994, p. 4423; Robinson et al. J. Med. Chem., 1996, p. 10; Keyes et al. J. Med. Chem., 1996, p. 508; Krise et al. J. Med. Chem., 1999, p. 3094; Rahmathullah et al. J. Med. Chem., 1999, p. 3994; Zhu et al. Bioorg. Med. Chem. Lett., 2000, p. 1121; Sun et al., J. Med. Chem., 2001, p. 2671; Ochwada et al., Bioorg. Med. Chem. Lett., 2003, p. 191; Sohma et al. Med. Chem., 2003, p. 4124; Ettmayer et al. J. Med. Chem., 2004, p. 2393; Stella et al., Adv. Drug Delivery Rev., 2007, p. 677, Josyula et al. International Patent Publication No. WO 2005/028473; Rhee et al. International Patent Publication No. WO 2005/058886, and EP 1,683,803. Following methods of these publications and refs. cited therein, respective prodrugs of the compounds of the present invention can be likewise prepared. Thus, prodrugs of compounds of the formula I are prepared by modifying functional groups present in a compound of the subject invention in such a way that the modifications may be cleaved in vivo to release the parent compound. Said prodrugs can be used, for example, to improve aq. solubility, oral, transdermal, or ocular bioavailability, to achieve a controlled (e.g., extended) release of the drug moiety, to improve tolerability, etc. Prodrugs include compounds of the subject invention wherein a hydroxy, sulfhydryl, amido or amino group in the compound is bonded to any group that may be cleaved in vivo to regenerate the free hydroxyl, amido, amino, or sulfhydryl group, respectively. Examples of prodrugs include, but are not limited to esters (e.g., acetate, formate, benzoate, phosphate or phosphonate derivatives), carbamates (e.g., N,N-dimethylaminocarbonyl), N-phosphoramides, of hydroxyl or amine-derived functional groups in compounds of the subject invention. Prodrug derivative can be used either as a neutral prodrug form (e.g. acid or amine), or a respective salt form thereof [e.g. sodium salt of a phosphate prodrug, or an amine salt (e.g. hydrochloride, citrate, etc.) for an amine group-bearing prodrug], or a zwitterionic form if both positively and negatively charged/ionizable functions are present. Prodrug groups may be incorporated at various sites of the formula I, provided that at least one appropriate functionality is available for a prodrug group installation.

Several preferred compounds of the present invention are illustrated below.

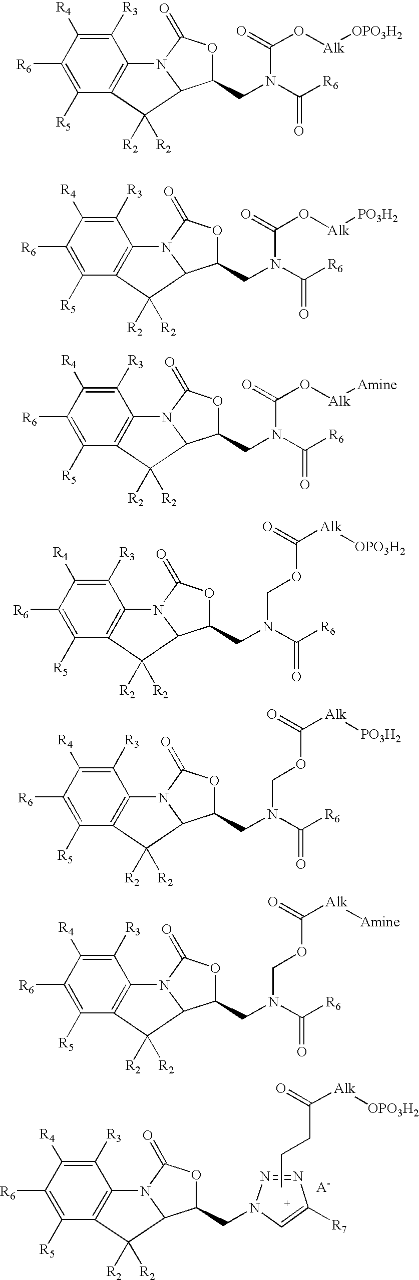

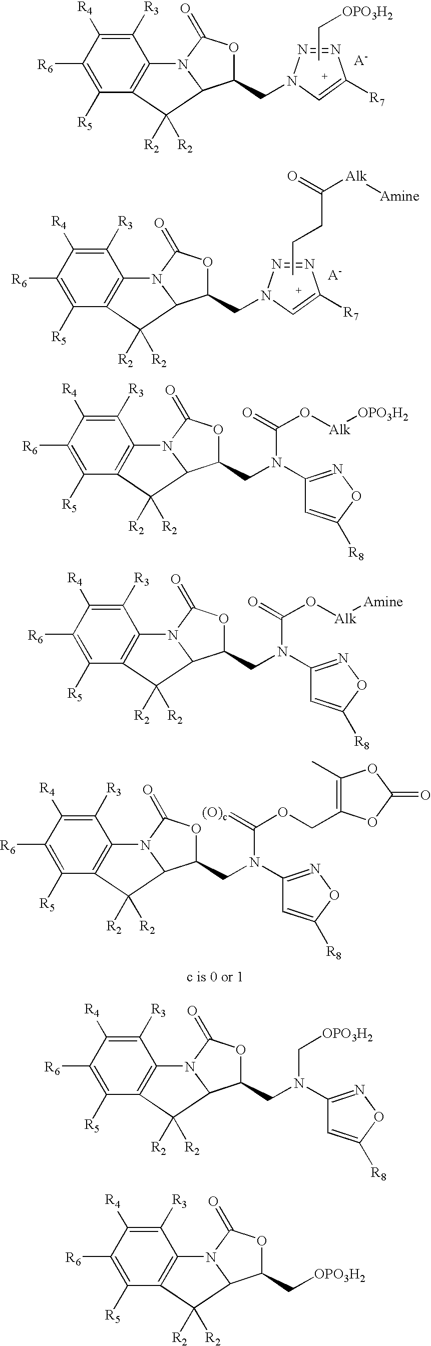

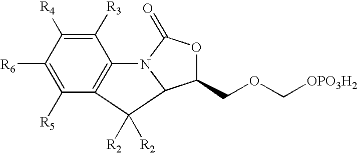

Additional preferred compounds of the present invention are illustrated below.

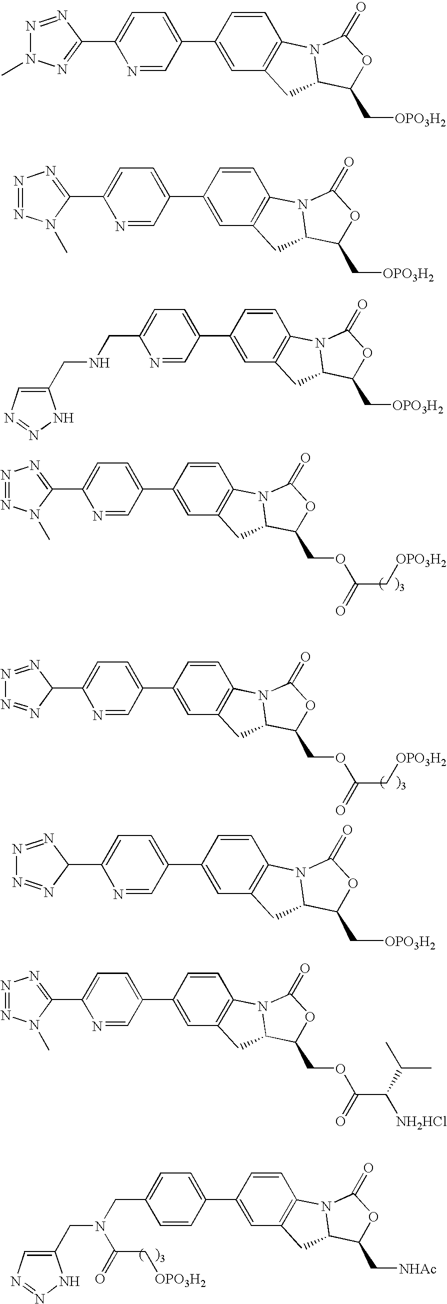

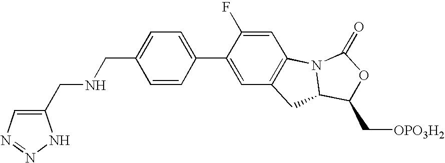

Additional preferred compounds of the present invention are illustrated below.

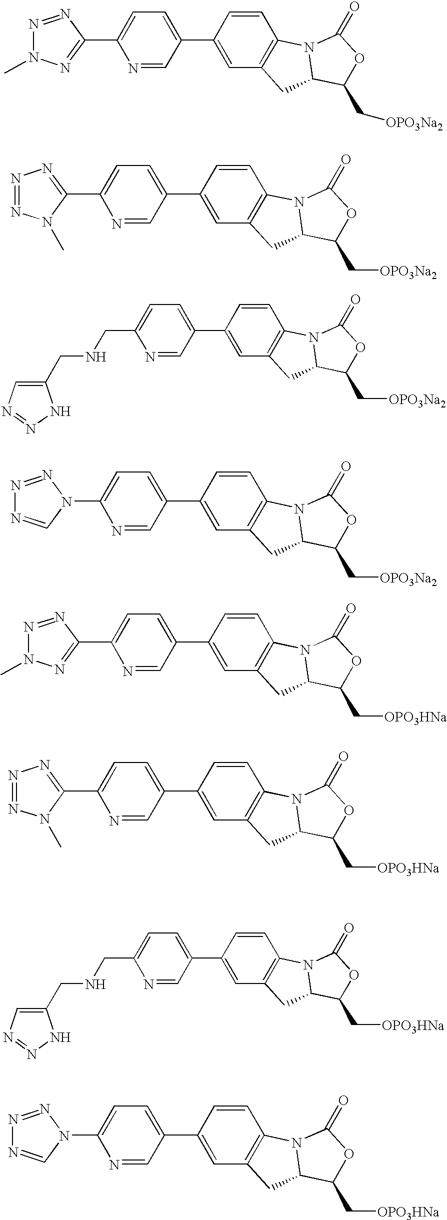

The term “mammal” refers to all mammals including humans, livestock, and companion animals.

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “r.t.” for room temperature).

### Illustrative Aspects

Within the broadest definition of the present invention, certain compounds of the compounds of formula I may be preferred. Specific and preferred values listed below for radicals, substituents, and ranges, are for illustration only; they do not exclude other defined values or other values within defined ranges for the radicals and substituents.

In some preferred compounds of the present invention C₁₋₄alkyl can be methyl, ethyl, propyl, isopropyl, butyl, iso-butyl, sec-butyl, and isomeric forms thereof.

In some preferred compounds of the present invention C₂₋₄alkenyl can be vinyl, propenyl, allyl, butenyl, and isomeric forms thereof (including cis and trans isomers).

In some preferred compounds of the present invention C₃₋₆ cycloalkyl can be cyclopropyl, cyclobutyl, cyclopentyl, cyclohexyl, and isomeric forms thereof.

In some preferred compounds of the present invention C₁₋₄ heteroalkyl can be hydroxymethyl, hydroxyethyl, and 2-methoxyethyl.

In some preferred compounds of the present invention halo can be fluoro (F) or chloro (Cl).

In some preferred compounds of the present invention, Z can be Het¹ or Het².

In some preferred compounds of the present invention R¹ can be CH₂NHC(═O)C₁₋₄alkyl or CH₂NHC(═O)OC₁₋₄alkyl.

In some preferred compounds of the present invention R¹ can be (4-R⁷-1,2,3-triazol-1-yl)methyl, (5-R⁷-isoxazol-3-yl)aminomethyl, or (5-R⁷-isoxazol-3-yl)oxymethyl, wherein R⁷ is H, C₁₋₃alkyl, halo, or CN.

In some preferred aspects, R¹ in a compound of formula A is CH₂OH, CH₂OPO₃H₂, CH₂OC(═O)CH₂CH₂CH₂OPO₃H₂, or CH₂OC(═O)CH(NH₂)C₁₋₄alkyl.

In some preferred aspects, group R¹ is CH₂OH.

In some preferred aspects, group R¹ is selected from CONH₂ or CONHMe.

In some preferred aspects, group R¹ is selected from CH₂NHC(═O)Me, CH₂NHC(═O)Et, or CH₂NHC(═O)OMe.

In some preferred aspects, group R¹ is selected from CH₂(1,2,3-triazol-1-yl) or CH₂(4-methyl-1,2,3-triazol-1-yl).

In some preferred aspects, group R¹ is selected from CH₂NH(isoxazol-3-yl), CH₂O(isoxazol-3-yl), CH₂NH(pyridin-2-yl), or CH₂O(pyridin-2-yl), CH₂NH(pyridin-3-yl), or CH₂O(pyridin-3-yl).

In some preferred aspects, group R¹ is selected from CH₂NH(isoxazol-5-yl) or CH₂O(isoxazol-5-yl),

In some preferred aspects, groups R², R³ and R⁴ are independently selected from H or F.

In some preferred aspects, group R² is H, and group R⁴ is F.

In some preferred aspects, R², R³, R⁴ and R⁵ independently can be H or F.

In some preferred aspects, R² is H; and R³, R⁴ and R⁵ are independently selected from H and F.

In some preferred aspects, Z in compounds of formula I is selected from Het¹ or Het².

In some preferred aspects, Z in compounds of formula I is selected from Het¹-CH₂—W—CH₂—, or Het²-CH₂—W—CH₂—, wherein W is NH, NC₁₋₅alkyl, NC(═O)OC₁₋₅alkyl, NC(═O)C₁₋₅alkyl, NC(═O)Het¹, O, S, S(O)ₙ, and wherein n is 0, 1, or 2.

In some preferred aspects, Het¹ can be 2-pyridyl, 3-pyridyl, 4-pyridyl, 3-isoxazolyl, 4-isoxazolyl, 5-isoxazolyl, 1,2,3-triazol-1-yl, 1,2,5-thiadiazol-3-yl, 1,3,4-oxadiazolyl-, 1,2,4-oxadiazolyl-, and isoxazolidin-3-yl group.

In some preferred aspects, Het² can be pyrrolyl, imidazolyl, pyrazolyl, 3-cyanopyrazolyl, 1,2,3-triazolyl, 3-cyano-1,2,3-triazolyl, 1,2,4-triazolyl, 1,2,3,4-tetrazolyl, and isoxazolidin-3-yl group.

It will also be appreciated by those skilled in the art that compounds of the present invention may have additional chiral centers and be isolated in optically active and racemic forms. The present invention encompasses any racemic, optically active, tautomeric, or stereoisomeric form, or mixture thereof, of a compound of the invention.

One preferred group of compounds of the present invention is illustrated by:

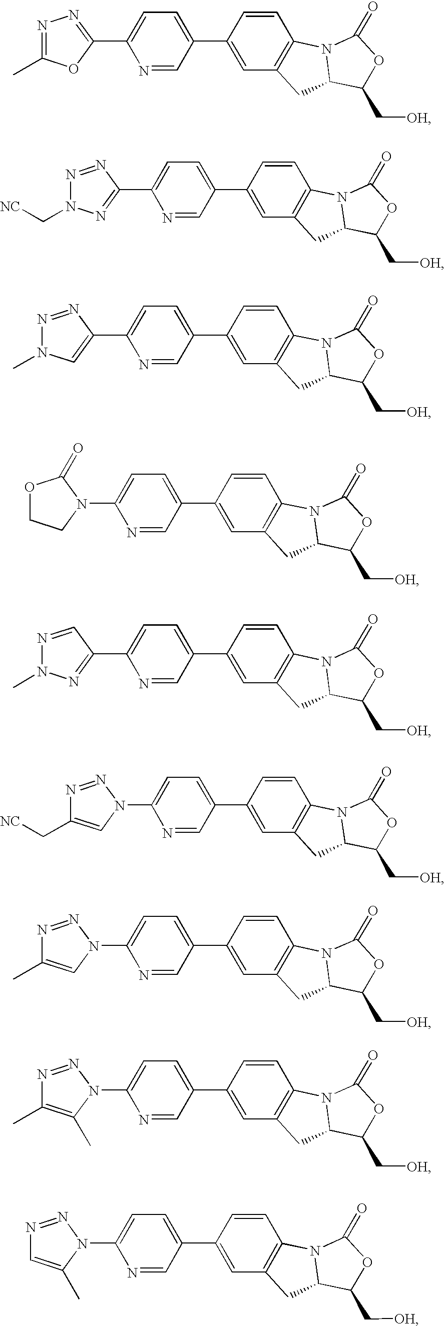

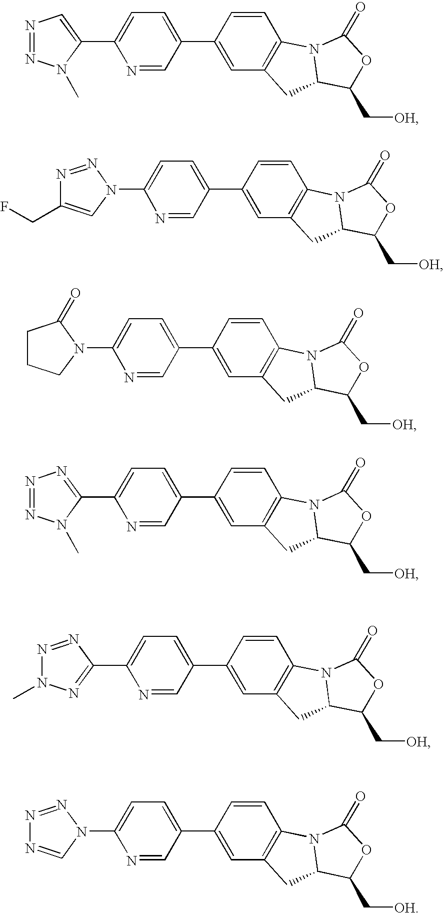

Another preferred group of compounds of the present invention is illustrated by:

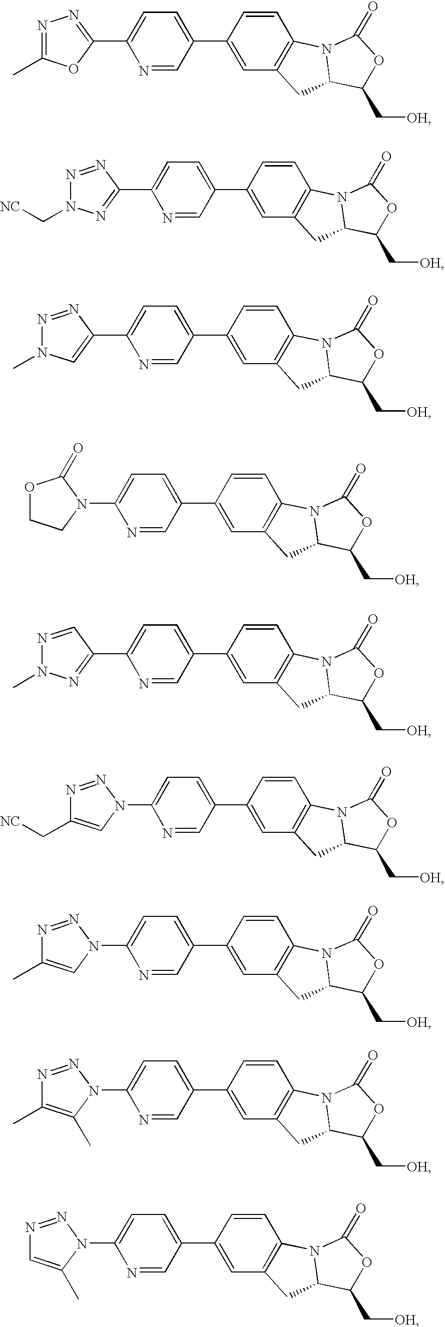

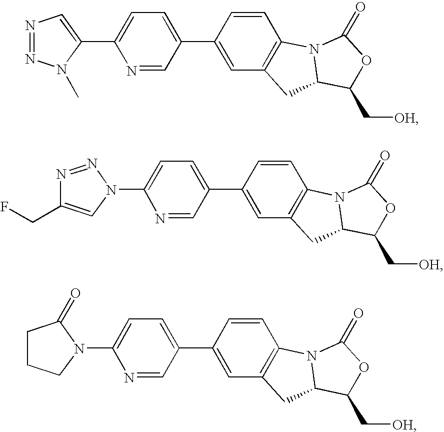

Another preferred group of compounds of the present invention is illustrated by:

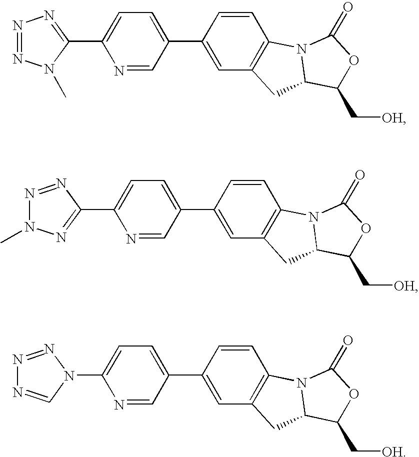

Another preferred group of compounds of the present invention is illustrated by:

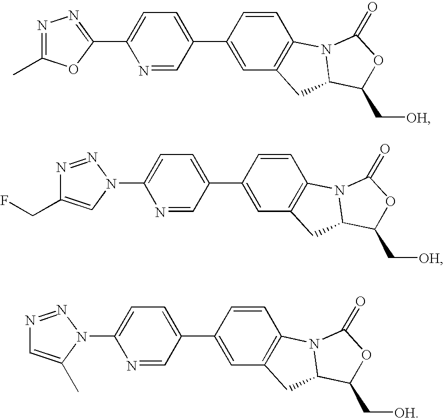

Another preferred group of compounds of the present invention is illustrated by:

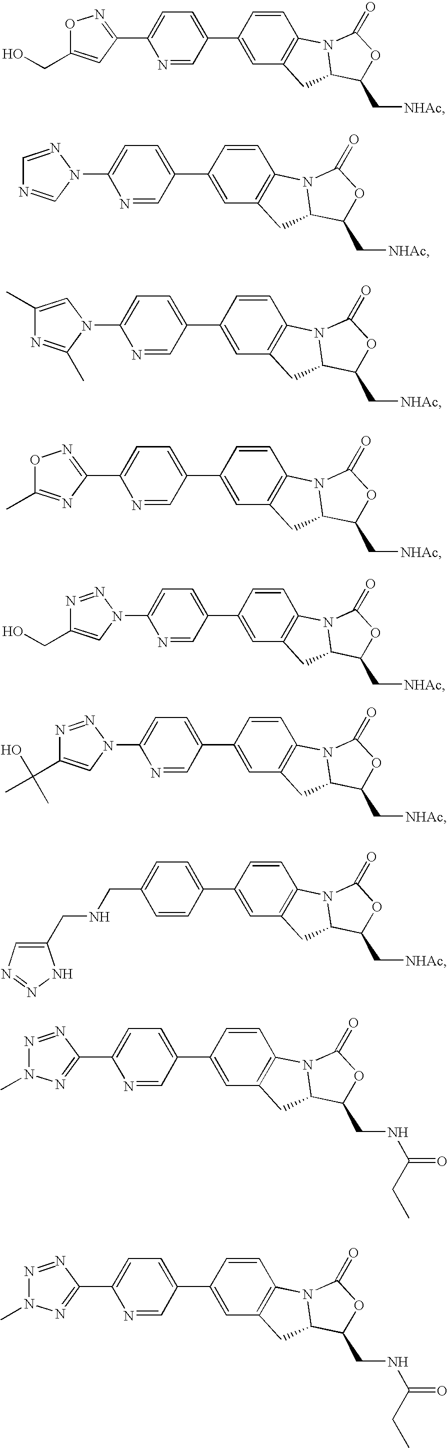

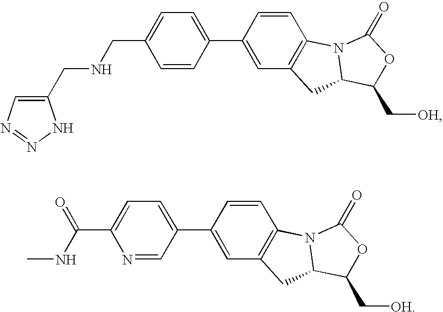

Another preferred group of compounds of the present invention is illustrated by:

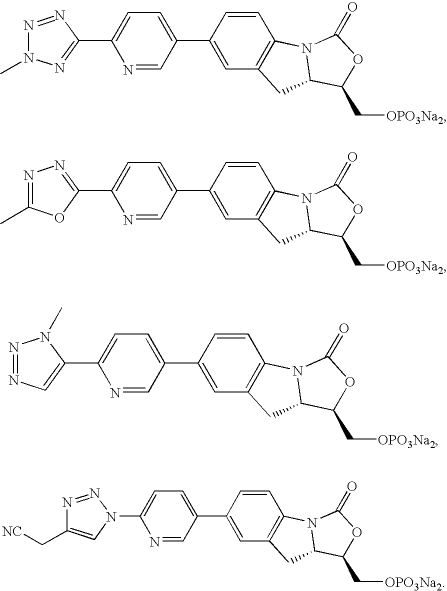

Another preferred group of compounds of the present invention is illustrated by:

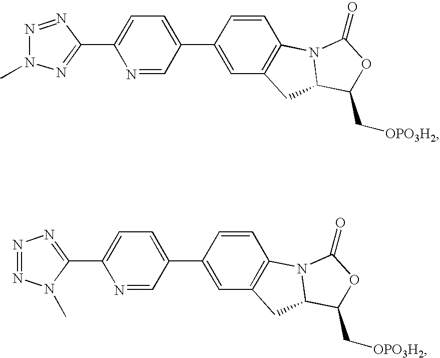

Another preferred group of compounds of the present invention is illustrated by:

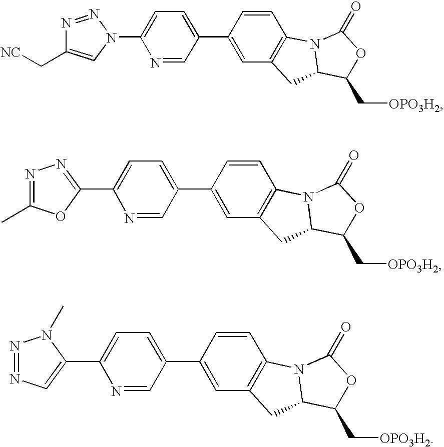

Another preferred group of compounds of the present invention is illustrated by:

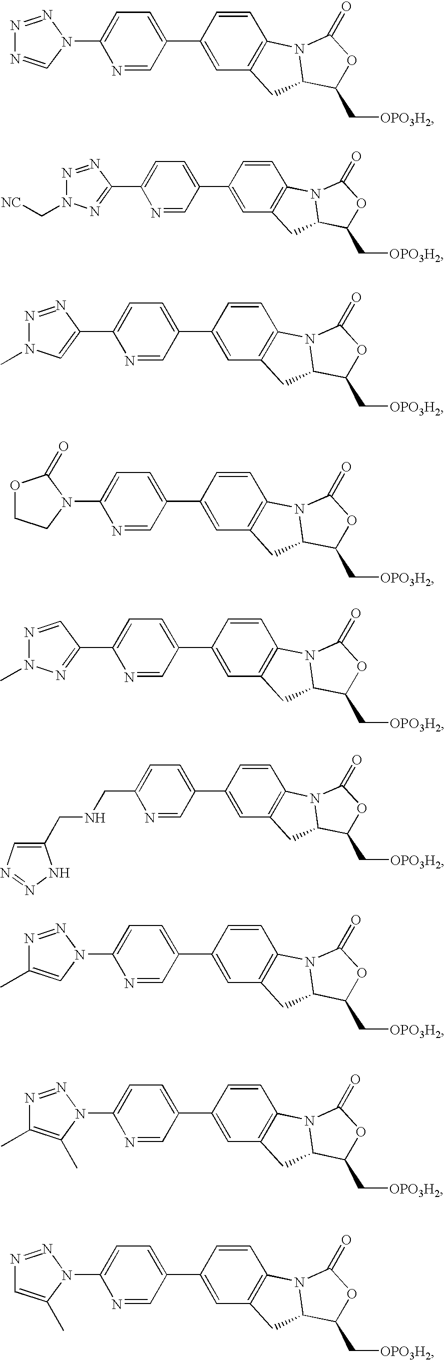

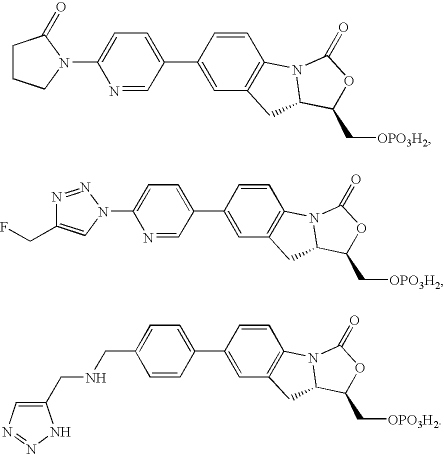

Another preferred group of compounds of the present invention is illustrated by:

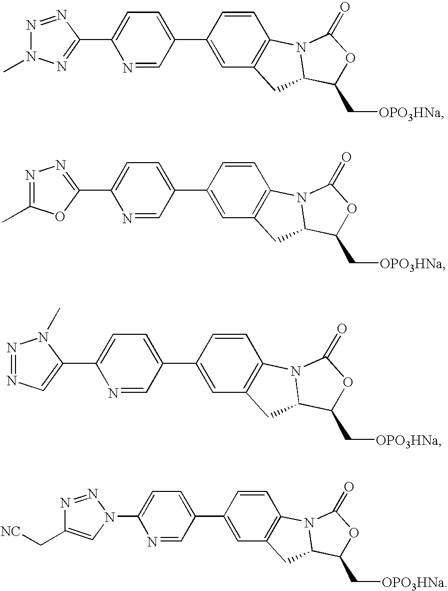

Another preferred group of compounds of the present invention is illustrated by:

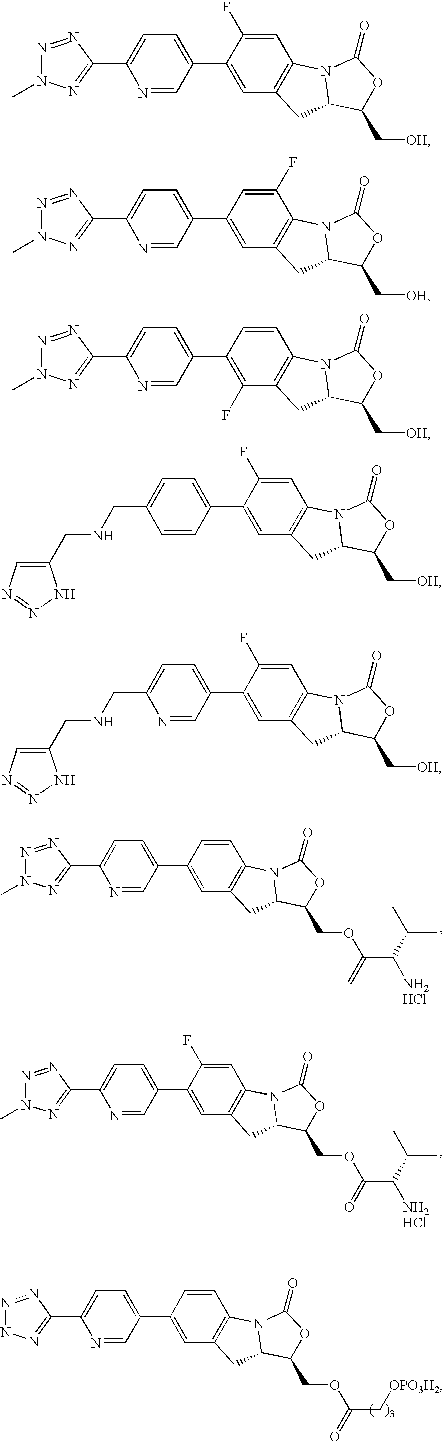

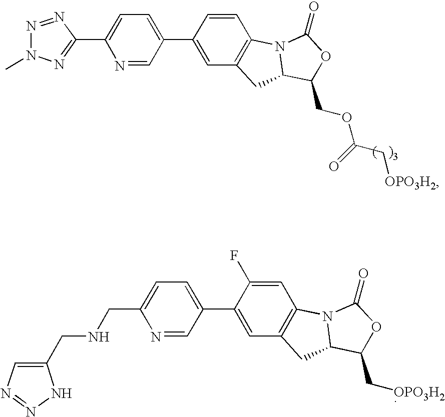

Another preferred group of compounds of the present invention is illustrated by:

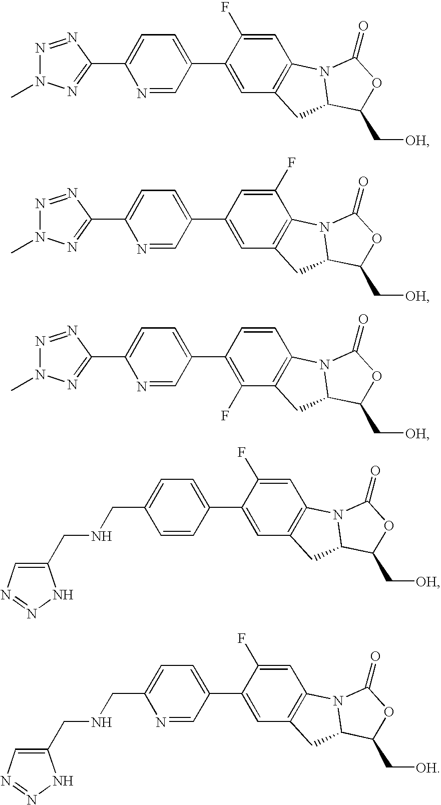

Another preferred group of compounds of the present invention is illustrated by:

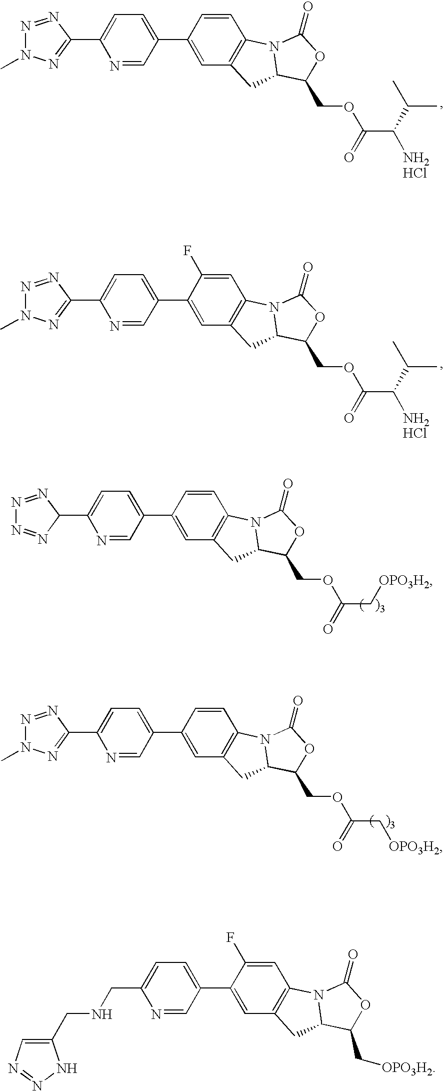

### General Synthetic Methods

The compounds of this invention can be prepared in accordance with one or more of Schemes discussed below. Synthesis of indoline compounds of this invention may generally follow known in the heterocyclic art methods described for certain other heterocyclic derivatives, including des-indoline compounds (i.e., those lacking the indoline structure). In one aspect of this invention, reagents more generally utilized for preparation of des-indoline heterocyclic compounds are intentionally replaced for a specific chemical(s) containing appropriate functionality(ies) amenable to a transformation into the indoline heterocyclic compounds invented herein. In another aspect of this invention, commercially available indoline derivatives or likewise reagents (prepared following general synthetic literature) are functionalized to install additional structural elements desired in the compounds of the present invention.

One general approach to the compounds of this invention is illustrated in general Scheme 1. Specific steps of illustrative Schemes below have a relevant analogy in the general synthetic heterocyclic art. Thus, various general transformations of carboxylic acids into aldehydes (analogous to steps (b) and (c) of Scheme 1) have been reviewed, for example, in Handbook of Reagents for Organic Synthesis: Oxidizing and Reducing Agents, John Wiley & Sons, 2000.

Methods for conversion of a carbonyl group into an olefinic group (analogous to step (d) of Scheme 1) have been more generally described, for example, in J. Amer. Chem. Soc., 1978, p. 3611; Chem. Lett. 1987, p. 2085; Chem. Eur. J., 2001, p. 4811; Chem. Commun., 2003, p. 442; and Tetrahedron, 2007, p. 8746.

General methods for oxidative transformation of olefinic compounds into diols (analogous to step (e) of Scheme 1) have been described, for example, in Handbook of Reagents for Organic Synthesis: Oxidizing and Reducing Agents, John Wiley & Sons, 2000.

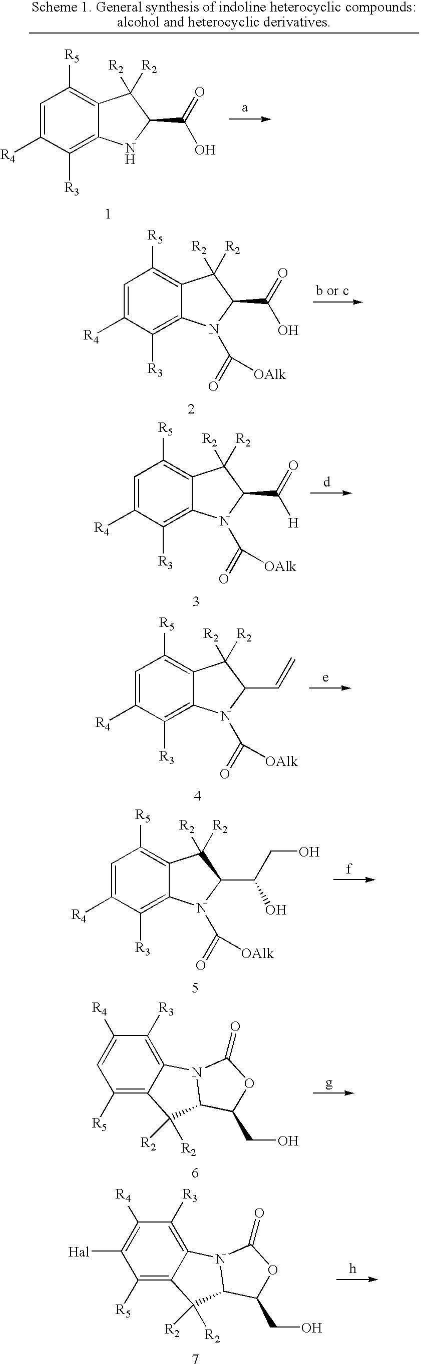

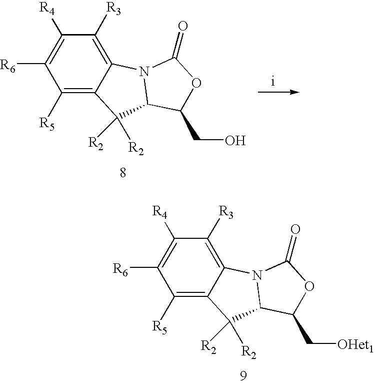

a) Carbamate forming reagent(s): AlkOC(═O)Cl, or AlkOCOC₆F₅, or alike; base: NaOH, NaH, Py, triethylamine (TEA) or alike; b) two sequential reactions: i) alcohol forming reducing agent(s): LiAlH₄ (LAH), or NaBH₄, or alike; ii) oxidizing reagent(s): oxalyl chloride/DMSO, or triphosgene/DMSO, or Py-SO₃, or Dess-Martin periodinane, or alike; c) two sequential reactions: i) Weinreb amide forming reagent(s): N,N′-carbonyldiimidazole (CDI), MeONHMe, base (Py, triethylamine (TEA) or alike); ii) reducing reagent(s): diisobutylaliminum hydride (DIBAL-H), or LAH, or alike; d) methylene forming reagent(s): Ph₃P═CH₂, or Tebbe's reagent, or CH₂I₂/Ti(OPr-i)₄/Zn, or CH₂I₂/Me₃Al/Zn, or alike; e) diol forming oxidant(s): N-methylmorholine N-oxide (NMO)/OsO₄, or K₂OsO₂(OH)₄/K₃Fe(CN)₆/MeSO₂NH₂, or Sharpless AD-mix-alpha or AD-mix-alpha reagent(s), MeSO₂NH₂, or alike; f) base: K₂CO₃, LiOH, TEA, 1,8-diazabicyclo[5.4.0]undec-7-ene (DBU), or alike; g) halogenating reagent(s): e.g., N-halosuccinimide, Br₂, tetrabutylammonium tribromide, or alike; h) arylating or heteroarylating reagent(s): Ar—B(OH)₂, or Ar—B(OAlk′)₂ or Het¹-B(OH)₂, or Het¹-B(OAlk′)₂, or Het²-B(OH)₂, or Het²-B(OAlk′)₂ selected from boronic acid, boronic acid ester (e.g. (picolinato)boron ester) or alike, Pd catalyst [e.g. PdCl₂(dppf)DCM, Pd(PPh₃)₄ or alike]; i) Het¹ OH or Het²OH, Mitsunobu reagents: e.g., Ph₃P, DIAD, base.

Methods for metal-mediated transformations analogous to step (h) of Scheme 1 have been more generally reviewed, for example, in Synthesis, 2004, p. 2419. The boron coupling chemistry illustrated for above step (h) may be supplanted by other metal-mediated couplings, such as tin-coupling chemistry similar to that more generally described, for example, in Tetrahedron Lett., 1988, p. 2135.

Another general synthesis of compounds of the present invention is illustrated by Scheme 2. The triazole forming chemistry analogous to step (c) of Scheme 2 has been more generally described, for example, in Org. Lett., 2008, p. 497.

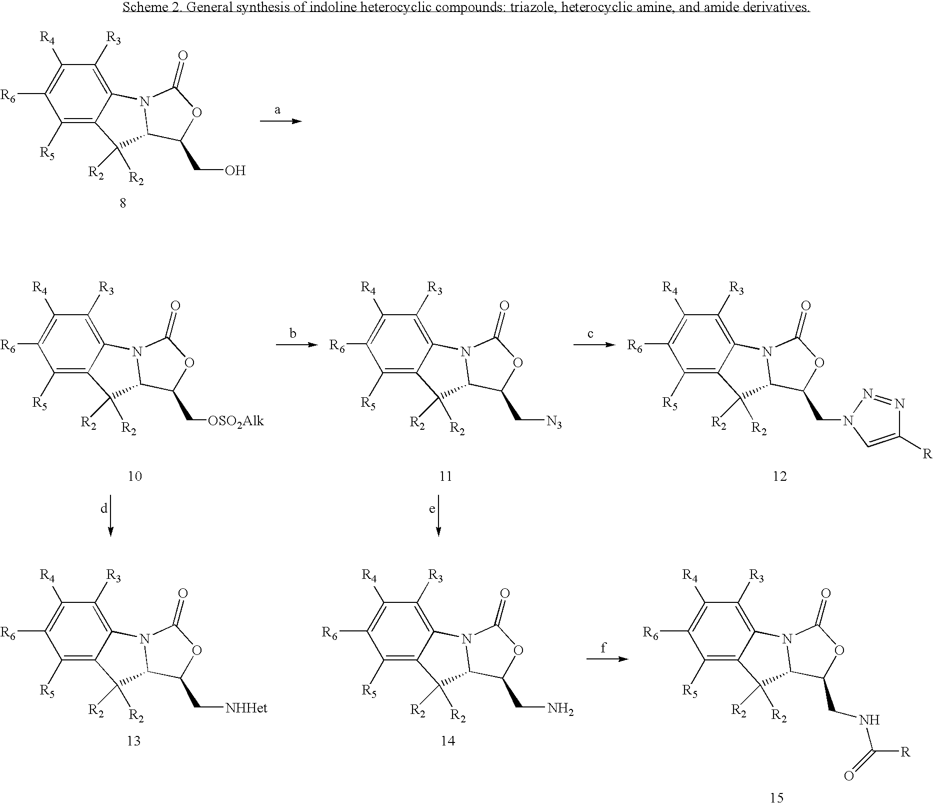

a) AlkSO₂Cl, base; b) NaN₃, LiN₃, or alike; optional TfN₃, base in place of steps (a) and (b); c) triazole forming reagent: e.g. R—C≡C—H, norbornadiene, or alike; d) HetNH₂, base; optional two-step sequence, e.g. i) 3-BocNH-isoxazole, NaH or KOBu-t; ii) HCl or trifluoroacetic acid; e) reducing agent(s): H₂/Pd/C, Ph₃P/water, or alike; f) acylating agent: e.g. RC(═O)Cl, RC(═)OC₆F₅, or RCOOH/HATU; base: K₂CO₃, TEA or alike.

Another general synthesis of compounds of the present invention is illustrated by Scheme 3.

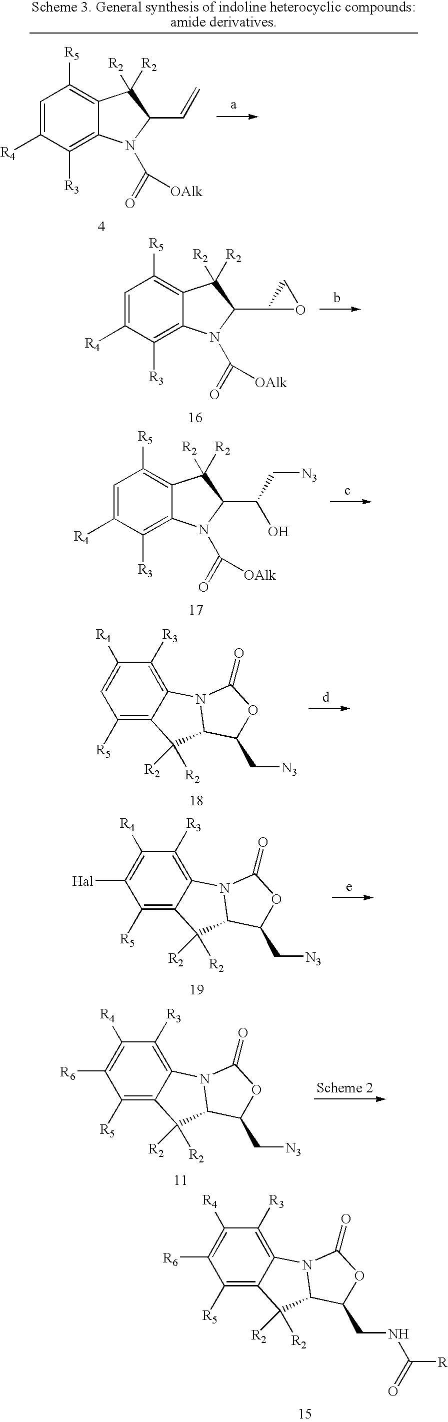

a) epoxide forming reagent(s): 3-ClC₆H₄CO₃H, or tert-BuOOH/Ti(OPr-i)₄/diethyl tartrate, or Oxone^{R}/ketone (e.g., acetone, or a chiral ketone) reagent; or Jacobsen epoxidation reagent(s); b) NaN₃, LiN₃, or alike; c) base: K₂CO₃, LiOH, TEA, 1,8-diazabicyclo[5.4.0]undec-7-ene (DBU), or alike; d) halogenating reagent(s): e.g., N-halosuccinimide, Br₂, tetrabutylammonium tribromide, or alike; e) arylating or heteroarylating reagent(s): Ar—B(OH)₂, or Ar—B(OAlk′)₂ or Het¹-B(OH)₂, or Het¹-B(OAlk′)₂, or Het²-B(OH)₂, or Het²-B(OAlk′)₂ selected from boronic acid, boronic acid ester (e.g. (picolinato)boron ester) or alike, Pd catalyst [e.g. PdCl₂(dppf)DCM, Pd(PPh₃)₄ or alike].

Sharpless asymmetric epoxidation analogous to step (a) of Scheme 3 has been more generally described, for example, in J. Amer. Chem. Soc., 1987, p. 5765. Synthesis of asymmetric epoxides using Oxone^{R}-chiral ketone oxidants is more generally described, for example, in J. Org. Chem., 2002, p. 2435.

Another general synthesis of compounds of the present invention is illustrated by Scheme 4. Analogous to steps (c) and (d) of Scheme 4, preparation of phosphate prodrug esters have been more generally described, for example, in Org. Proc. Res. Dev., 2002, p. 109.

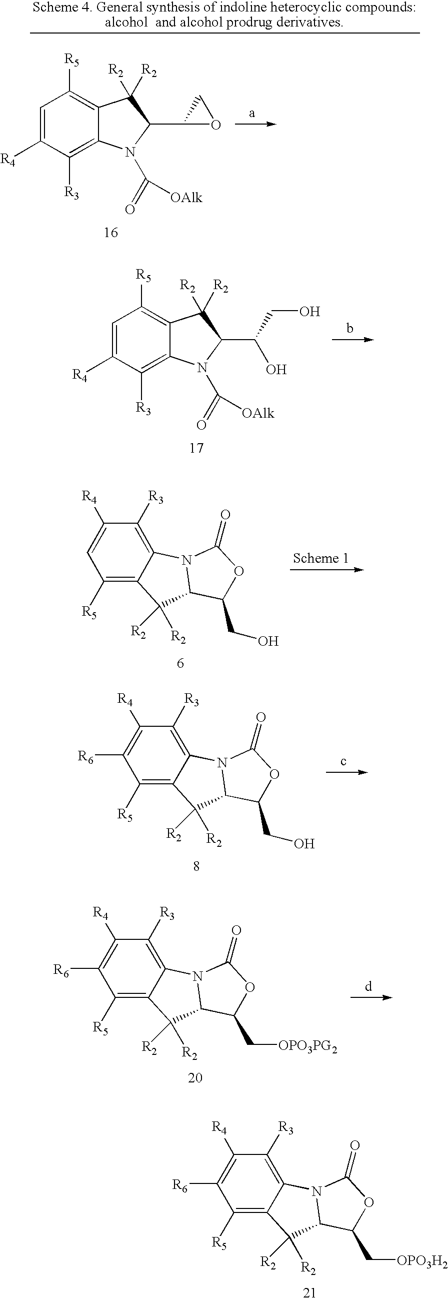

a) aqueous base (e.g., K₂CO₃ or LiOH) or acid (e.g., H₂SO₄, or acidic ion-exchange resin); b) base: K₂CO₃, LiOH, TEA, DBU, or alike; c) protected pyrophosphate reagent (e.g., tetrabenzylpyrophosphate or tetra(tert-butyl)pyrophosphate, or CIP(═O)(OPG)₂, wherein PG is a protective group, base (e.g., NaH, DBU, or alike); or reagents Alk₂NP(OPG)₂, tetrazole, 3-ClC₆H₄CO₃H; d) deprotective reagent(s): e.g., for PG=benzyl: H₂/Pd/C; for PG=tert-Bu: TFA or HCl.

Another general synthesis of compounds of the present invention is illustrated by examples of Scheme 4a. As may be required for a specific therapeutical application, either mono- or di-basic salts 21a could be prepared via a conventional acid-base neutralization chemistry. For example, 1 equivalent of a compound 21 with 1 equivalent of a basic reagent (e.g. inorganic base such as NaHCO₃ or NaOAc, or an organic or inorganic amine base) is generally used to prepare a mono-basic salt (e.g., mono-sodium salt 21a, wherein one of P¹ is H, and another P² is a sodium group, to form the mono-basic salt 21a bearing the group OPO₃HNa). Likewise, 1 equivalent of compound 21 with 2 equivalents of a basic reagent is generally used to prepare a di-basic salt 21a (such as disodium phosphate 21a bearing the group OPO₃Na₂). A multitude of basic reagents without limitation may be generally used to form such derivatives 21a.

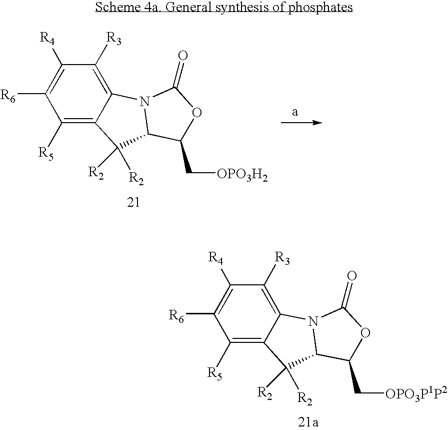

a) inorganic base (e.g. alkali metal base such as NaOH, NaOAc, Na₂CO₃, or NaHCO₃) or an amine base (e.g., ammonia or ammonium acetate, or triethylamine) such that the resulted salt group —OPO₃P¹P² is either mono- or dibasic salt of alkali metal (e.g. sodium) or of amine (e.g. OPO₃P¹P² is OPO₃HNa or OPO₃Na₂).

Likewise, using a fractional amount of a base generally allows a preparation of a mixture of a mono-basic salt 21a and a di-basic salt 21a (when generally less than 2 equivalents of base are used for 1 equivalent of an acid 21), or a mixture of the mono-basic salt 21a with an acidic compound 21 as may be required (when generally less than 1 equivalents of base are used for 1 equivalent of an acid 21). These general methods allow to adjust the desired salt composition and the acidity or basicity (pH) of the resulted pharmaceutical ingredient as could be required for specific therapy and/or storage.

Another general synthesis of compounds of the present invention is illustrated by Scheme 5. General methods for preparation of cyanohydrine derivatives analogous to the chemistry of step (a) of Scheme 5 have been reviewed, for example, in Angew. Chemie, 2004, p. 2753. Likewise, an optically active auxiliary or a catalyst can be optionally employed to produce a desired cyanohydrine isomer in a single step. Thus, a chiral catalytic system (e.g. taddol—Ti(IV), tartrate—Ti(IV), or triol—Ti(IV) complexes, or asymmetric boron, aluminum, tin, or zinc catalyst, or alike) can be employed, just as more generally described in Angew. Chemie, 2004, p. 2753.

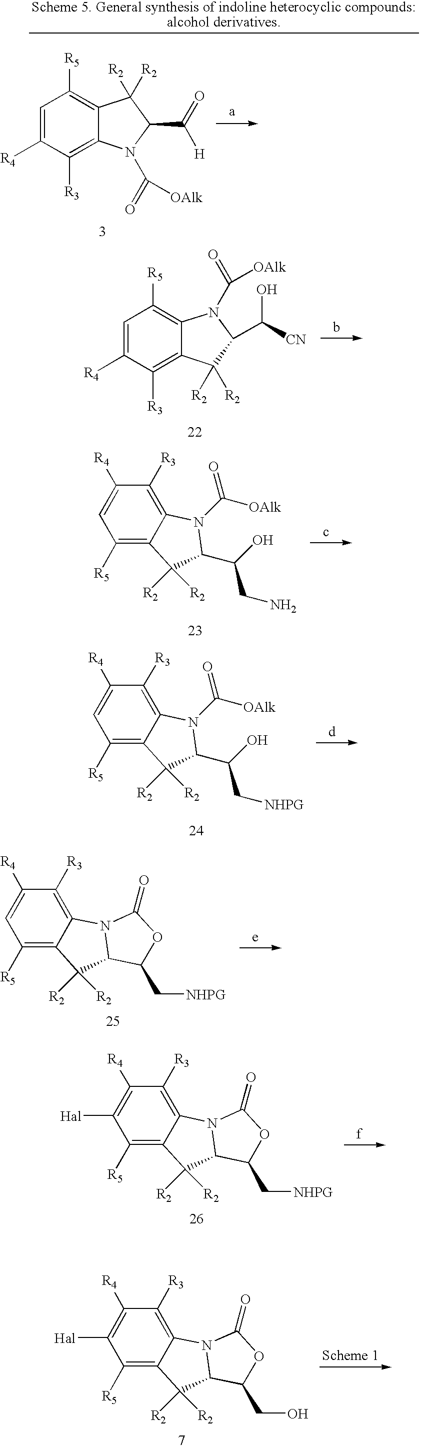

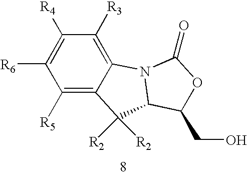

a) cyanohydrine forming reagent(s): KCN, or HCN, or Me₂C(OH)CN, or alike; or 2-step sequence: i) TMSCN, LiF; ii) trifluoroacetic acid (TFA), or tetrabutylammonium fluoride (TBAF), or alike; b) reducing reagent(s): BH₃ Me₂S or H₂, Pt/C or Pd/C, or alike; c) protecting reagents: Boc₂O, or Trt-Hal/base (e.g., K₂CO₃), or Cbz-Cl/base (e.g. Py), or alike; d) base: K₂CO₃, LiOH, TEA, DBU, or alike; e) halogenating reagent(s): e.g., N-halosuccinimide, Br₂, tetrabutylammonium tribromide, or alike; f) sequence of two reactions: i) deprotective reagent(s): TFA for PG=Boc or Trt, or H₂/Pt/C for PG=Cbz; ii) nitrous acid reagent(s): AlkONO/acid (e.g., TFA or AcOH), or NaNO₂/acid (e.g. aq. H₂SO₄), or alike; then optional base (e.g. aq. LiOH or alike).

Optionally, the substituent R⁶ can be installed into the requisite indoline reagent prior to transformations illustrated by Schemes 1-5 or non-critical variations thereof, except that no coupling step to replace the Hal group for R⁶ group is then required.

Certain fluorinated compounds invented herein can be made either using respective fluorine-substituted starting materials per Schemes 1-5 (if one, two, or all of R³, R⁴, or R⁵ are F), or produced via a direct fluorination of appropriate indoline derivatives or precursor thereof. This process may be affected, for example, using electrophilic fluorinating reagents such as Selectfluor^{R} (generally described in J. Fluorine Chem., 2004, p. 543), N-fluorobenzenesulfonamide (generally described in Aldrichimica Acta, 1995, vol. 28, p. 36), CF₃OF (generally described in J. Am. Chem. Soc., 1980, p. 4845), N-fluoropyridinium salts (generally described in J. Am. Chem. Soc., 1990, p. 8563), or alike reagents.

Additional detailed synthetic schemes for the syntheses of specific compounds of the present invention are illustrated by methods described for Examples below.

The invention also contemplates exemplary synthesis, formulation, and methods of use, such as those described in, for example, publication PCT WO 2008/108988, the contents of which are hereby incorporated by reference herein.

### EXAMPLES

Embodiments of the present invention are described in the following examples, which are meant to illustrate, not to limit the scope of this invention. Common abbreviations well known to those with ordinary skills in the synthetic art used throughout. ¹H NMR spectra (δ, ppm) are recorded in CDCl₃ unless specified otherwise. Mass-spectroscopy data for a positive ionization method are provided. Chromatography means silica gel chromatography unless specified otherwise. TLC means thin-layer chromatography, and PTLC means preparative TLC. Unless specified otherwise, all reagents were either from commercial sources, or made by conventional methods described in available literature.

### Example 1

### Compound of Structure

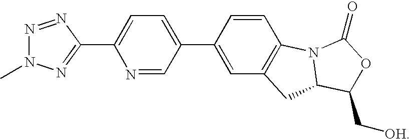

Scheme for Compound of Example 1:

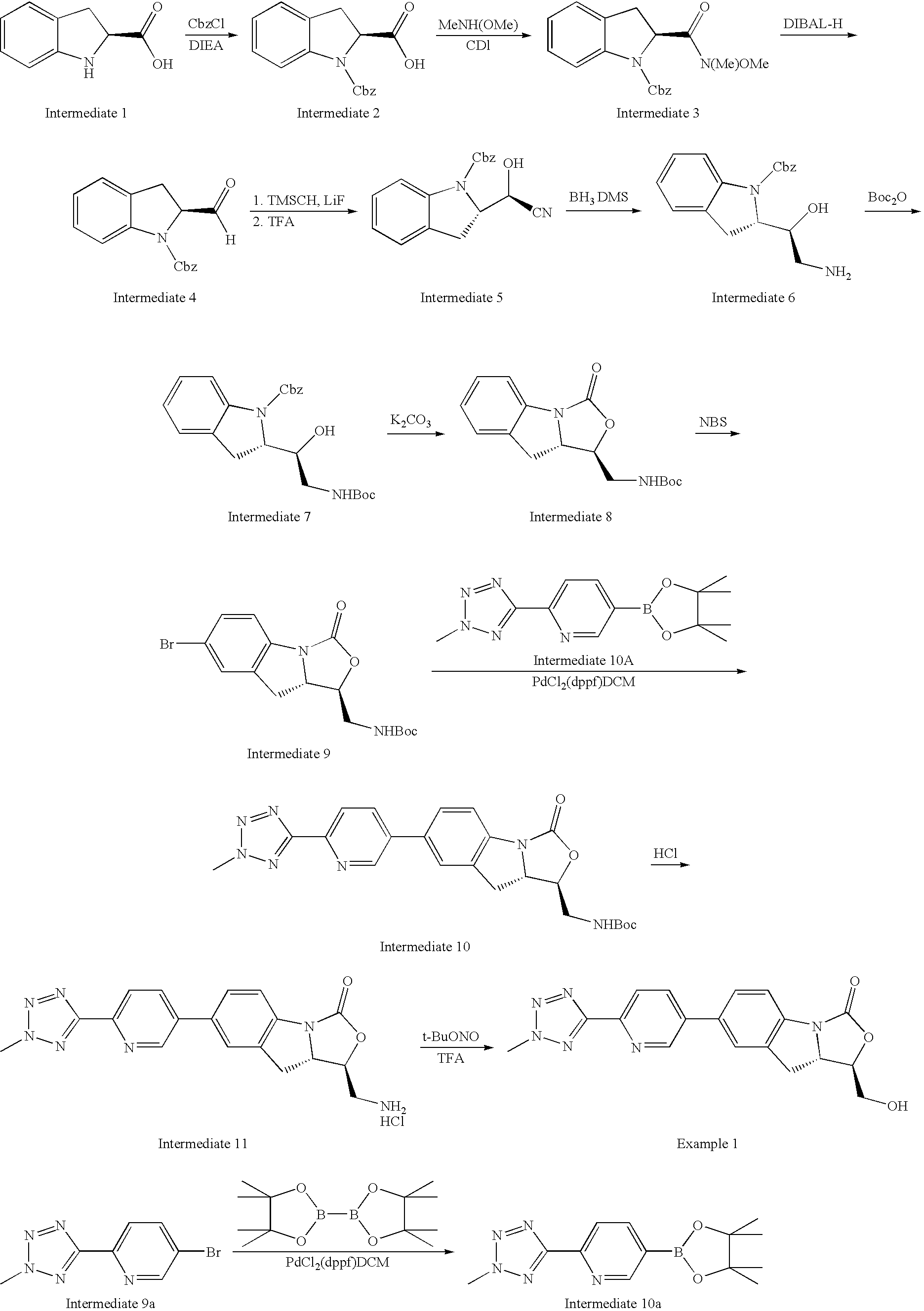

Intermediate 2. Cbz-Cl (20 mL, 0.13 mol) in MeCN (50 mL) was added dropwise to Intermediate 1 (20 g, 0.12 mol) and DIEA (43 mL, 0.25 mol) in MeCN (350 mL) at 5-10° C. over 20 min. The mixture was allowed to warm up r.t. After 3 h, volatiles were removed under vacuum. The oily residue was dissolved in EtOAc and washed with 1% aq. HCl, water, brine, and dried (MgSO₄). Solvent was removed under vacuum to afford the product as thick oil that crystallized in refrigerator into a brownish solid.

Intermediate 3. CDI (14.2 g, 0.087 mol) was added to Intermediate 2 (20 g, 0.067 mol) in DCM (150 mL) at −5° C., and the solution was kept at −5° C. for 1 h. DIEA (15.3 mL, 0.087 mol) was added, followed by N,O-dimethylhydroxylamine hydrochloride (8.5 g, 0.087 mol). The mixture was allowed to warmed up to r.t. and stirred for 30 min, then filtered and washed with EtOAc to obtain the product as a white solid.

Intermediate 4. DIBAL-H in toluene (353 mL, 0.35 mol) was added dropwise with stirring under Ar to Intermediate 3 (40 g, 0.12 mol) in dry THF (800 mL) at −78° C. over 30 min. The reaction mixture was stirred at −78° C. for 1 h, then allowed to warm up to −30° C. over 1 h and stirred for an additional 30 min. Cold MeOH (20 mL) was added, then 2M HCl (200 mL) was added. The product was extracted with EtOAc (1000 mL). The organic layer was washed with 2M HCl (2×200 mL) and brine (200 mL), and dried (MgSO₄). Solvent was evaporated under vacuum. Purification by column chromatography (petroleum ether:EtOAc=8:1) afforded the product as a yellow solid.

Intermediate 5. Intermediate 4 (25.9 g, 0.092 mol) and LiF (3.5 g, 0.183 mol) were dissolved in THF (200 mL) under Ar, then TMSCN (17 mL, 0.183 mol) was added with stirring. The resulting was stirred at r.t. for 5 h under Ar. Volatiles were removed under vacuum, and the crude material was dissolved in THF (ca. 200 mL). TFA (38 mL) was added dropwise with stirring, and the mixture was stirred at r.t. for 12 h. Excess of EtOH was added, and the volatiles removed under vacuum. Purification by chromatography (silica gel, petroleum ether:EtOAc, 8:1) afforded a solid, which was crystallized with petroleum ether:EtOAc (12:1) to afford the product. MS (m/z): 309 [M+H].

Intermediate 6. 2 M Borane-dimethylsulfide in THF (24 mL, 48 mmol) was added to a solution of Intermediate 6 in dry THF (50 mL) at r.t. The reaction mixture was heated to reflux and stirred for an additional 30 min, then cooled to r.t. and carefully quenched with MeOH. Volatiles were removed under vacuum, and the crude product directly used in the next step.

Intermediate 7. To a solution of the crude amine Intermediate 6 (17.53 mmol, 1.0 equiv) in DCM (85 mL) was added (Boc)₂O (5.73 g, 26.3 mmol, 1.5 equiv), and the mixture was stirred at r.t. until the disappearance of the starting amine. The mixture was diluted with DCM (50 mL) and then washed with ca. 1% aq. HCl. Organic layer was dried (MgSO₄) and evaporated under vacuum. The crude product was purified by column chromatography (gradient: hexanes-EtOAc).

Intermediate 8. To a solution of Intermediate 7 (13.5 g, 32.8 mmol, 1.0 equiv) in CH₃CN (150 mL) was added K₂CO₃ (4.52 g, 32.77 mmol, 1.0 equiv), and the mixture was stirred at 45° C. for 12 h, then filtered. The precipitate was washed with EtOAc. Solvent was removed under vacuum, and the crude product purified by column chromatography (gradient hexanes/EtOAc).

Intermediate 9. To a solution of Intermediate 8 (6.0 g, 19.73 mmol, 1.0 equiv) in MeCN (60 mL) were added N-bromosuccinimide (NBS; 4.57 g, 25.66 mmol, 1.3 equiv) and benzoyl peroxide (477 mg, 1.97 mmol, 0.1 equiv), and the mixture was stirred at r.t. o.n. Solvent was removed under vacuum, and the product was purified by column chromatography (gradient hexanes-EtOAc from 2.5% to 30% EtOAc).

Intermediate 10a. To the solution of Intermediate 9a (200 mg, 0.83 mmol, prepared as described in PCT WO 2005/058886) in dioxane (2 mL) was added pinacol diborane (270 mg, 1.06 mmol), KOAc (270 mg, 2.75 mmol) and PdCl₂(dppf)DCM (60 mg, 0.07 mmol), degassed and protected with N2. The mixture was stirred at 80° C. for 2-3 h. The reaction mixture was diluted with DCM (100 mL) and washed with brine (2×100 mL), dried (Na₂SO₄) and evaporated under vacuum, then purified by preparation TLC to give 2-(1-methyl-1H-tetrazol-5-yl)-5-(4,4,5,5-tetramethyl-1,3,2-dioxaborolan-2-yl)pyridine as white solid (160 mg). ¹H NMR (300 MHz, CDCl₃, ppm): 9.07 (s, 1H), 8.22 (m, 2H), 4.58 (s, 3H), 1.39 (s, 12H).

Intermediate 10. Intermediate 10a (352 mg, 1.22 mmol), Intermediate 9 (426 mg, 1.11 mmol) were dissolved in 1,4-dioxane (16 ml). The solution was flushed with N2 and then K₂CO₃ (338 mg, 2.45 mmol), and PdCl₂(dppf) DCM (91 mg, 0.11 mmol) were added. The mixture was heated at 80° C. for 7 h. The reaction was cooled to r.t., filtered, and the filtered residue was washed twice with DCM and then EtOAc. The combined filtrate was concentrated in vacuo, and then chromatographed on a silica gel column eluting with 5% MeOH/DCM to afford the product. ¹H NMR (300 MHz, CDCl₃, ppm): 8.91 (s, 1H), 8.26 (d, J=8.10 Hz, 1H), 7.98 (d, J=8.10 Hz, 1H), 7.52-7.47 (m, 3H), 5.13 (t, 1H), 4.63-4.59 (m, 2H), 4.46 (s, 3H), 3.70-3.54 (m, 2H), 3.43-3.34 (m, 2H), 1.29 (s, 9H). MS (m/z): 464 [M+H].

Intermediate 11. Intermediate 10 (200 mg, 0.43 mmol) was treated with 2N HCl in ether (15 ml), and stirred at r.t. for 3 h. Upon reaction completion, the mixture was concentrated to dryness to afford the crude product. MS (m/z): 364 (M+1).

Compound of Example 1. Intermediate 11 (90 mg, 0.25 mmol) in 1N TFA in 1,4-dioxane (9 ml) was cooled down with ice-water until 1,4-dioxane started to solidify, then t-BuONO (300 μl, 2.52 mmol) was added dropwise with stirring. The reaction was stirred at this temperature for 5 min, then at r.t. for 7 h. The reaction was then quenched with water (3 mL). The solvent was removed under vacuum, and the residue distributed between with EtOAc and water, and then extracted with EtOAc. The organic layers were combined, and concentrated under vacuum. The crude material was purified by preparative TLC eluting with 5-8% MeOH/DCM to afford the product. ¹H NMR (300 MHz, CD₃OD, ppm): 8.55 (s, 1H), 7.91 (d, J=8.10 Hz, 1H), 7.76 (dd, J=8.1 and 2.4 Hz, 1H), 7.24-7.22 (m, 2H), 7.14 (m, 1H), 4.48-4.40 (m, 1H), 4.32-4.29 (m, 1H), 4.14 (s, 3H), 3.64-3.49 (m, 2H), 3.19-3.06 (m, 1H), 2.92-2.82 (m, 1H). MS (m/z): 365 [M+H].

### Example 2

### Compound of Structure

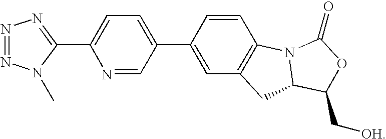

Scheme for Compound of Example 2:

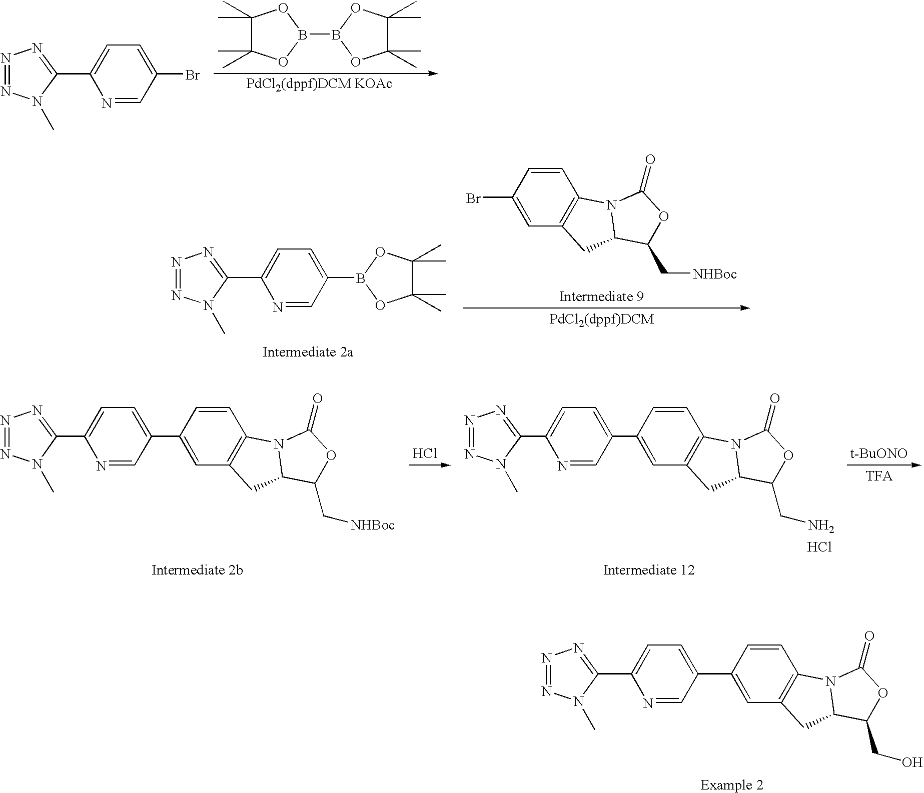

Intermediate 2a. Intermediate 2a was prepared analogously to the procedure for Intermediate 10a just as described in the preparation of Compound of Example 1, except using 5-bromo-2-(1-methyl-1H-tetrazol-5-yl)pyridine (prepared as described in PCT WO 2005/058886) instead of Intermediate 9a. ¹H NMR (300 MHz, CDCl₃, ppm): 9.03 (d, J=1.8 Hz, 1H), 8.27 (dd, J=8.4 and 1.8 Hz, 2H), 4.52 (s, 3H), 1.39 (s, 12H).

Intermediate 2b. Intermediate 2b was prepared analogously to the procedure for Intermediate 10 just as described in the preparation of Compound of Example 1, except using Intermediate 2a instead of Intermediate 10a.

Intermediate 12. Intermediate 12 was prepared analogously to the procedure for Intermediate 11 just as described in the preparation of Compound of Example 1, except using Intermediate 2b instead of Intermediate 10. MS (m/z): 364 (M+1).

Compound of Example 2. The compound of Example 2 was prepared analogously to the procedure for Compound of Example 1, except that Intermediate 11 was substituted for Intermediate 12. ¹H NMR (300 MHz, CD₃OD, ppm): 8.93 (d, J=1.8 Hz, 1H), 8.30 (d, J=8.10 Hz, 114), 8.09 (dd, J=8.1 and 2.4 Hz, 1H), 7.54-7.47 (m, 3H), 4.82-4.74 (m, 1H), 4.66-4.58 (m, 1H), 4.52 (s, 311), 3.98-3.81 (m, 2H), 3.44-3.36 (m, 1H), 3.28-3.18 (m, 1H). MS (m/z): 365 [M+H].

### Example 3

### Compound of Structure

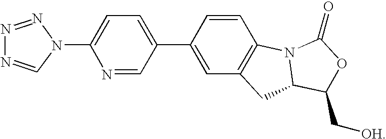

Scheme for Compound of Example 3:

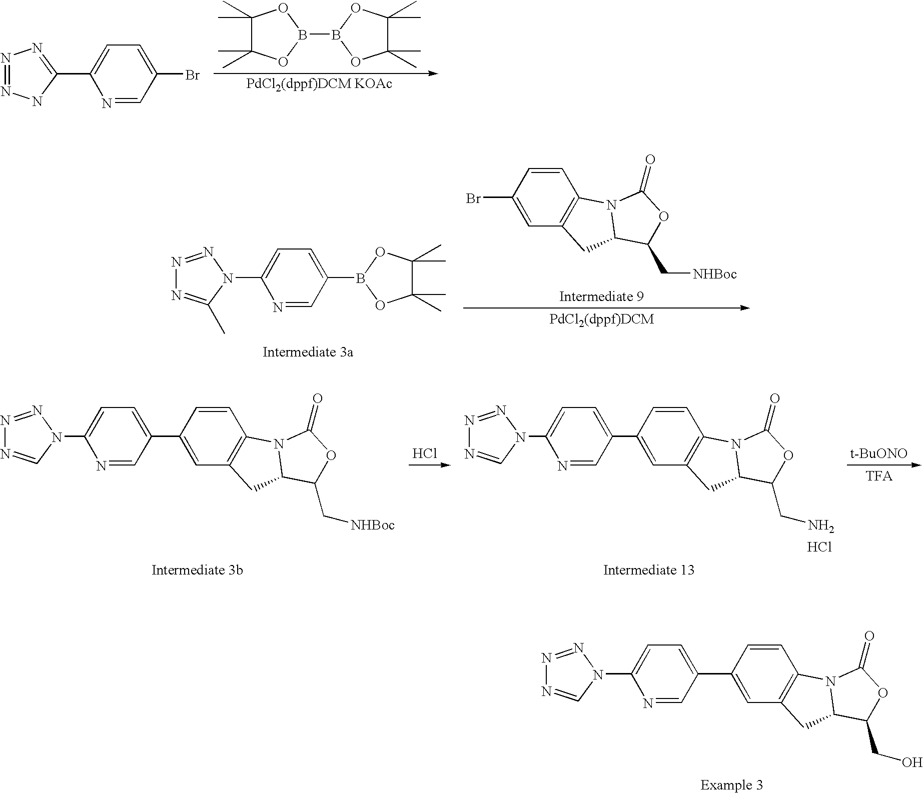

Intermediate 3a. To the solution of 5-bromo-2-(1,2,3,4-tetrazol-1-yl)pyridine (80 mg, 0.36 mmol) in dioxane (3 ml) was added pinacol diborane (100 mg, 0.39 mmol), KOAc (100 mg, 1.08 mmol) and PdCl₂(dppf)DCM (10 mg, 0.01 mmol). The reaction mixture was degassed and protected by N², then stirred at 80° C. for 4 h. The reaction mixture was diluted with DCM (100 mL), filtered, and evaporated under vacuum to give a yellow solid, which was purified by preparative TLC (5-10% Mesh in DCM) to give Intermediate 3a as white solid (48 mg). ¹H NMR (300 MHz, CDCl₃, ppm): 9.58 (s, 1H), 8.83 (m, 1H), 8.32 (dd, J=8.4 and 1.8 Hz, 1H), 8.06 (d, J=8.70 Hz, 1H), 1.38 (s, 12H).

Intermediate 3b. Intermediate 3b was prepared analogously to the procedure for Intermediate 10 just as described in the preparation of Compound of Example 1, except using Intermediate 3a instead of Intermediate 10a.

Intermediate 13. Intermediate 13 was prepared analogously to the procedure for Intermediate 11 just as described in the preparation of Compound of Example 1, except using Intermediate 3b instead of Intermediate 10.

Compound of Example 3. This compound was prepared analogously to the procedure to Compound of Example 1, except that Intermediate 11 was substituted for Intermediate 13. ¹H NMR (300 MHz, CD₃OD, ppm): 9.76 (s, 1H), 8.74 (d, J=1.8 Hz, 1H), 8.22-8.11 (m, 2H), 7.57-7.47 (m, 3H), 4.79-4.74 (m, 1H), 4.67-4.62 (m, 1H), 4.52 (s, 3H), 3.99-3.81 (m, 2H), 3.46-3.37 (m, 1H), 3.28-3.19 (m, 1H). MS (m/z): 351 [M+H].

### Example 4

### Compound of Structure

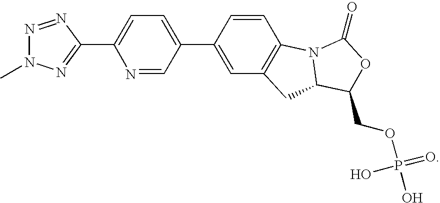

Scheme for Compound of Example 4:

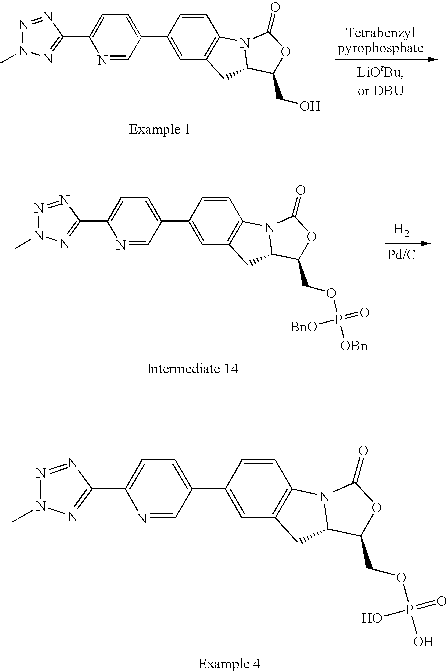

Intermediate 14.

Method A: The solution of Compound of Example 1 (60 mg, 0.16 mmol) and tetrabenzyl pyrophosphate (TBPP; 120 mg, 0.22 mmol) in THF (12 ml) was flushed with N², and cooled down with dry ice-acetone for 15 min, then was added 1 M LiOBu-t in THF (0.42 mL, 0.42 mmol). After the reaction was maintained at this temperature for 3 h, it was quenched with saturated NH₄Cl aq., and extracted with EtOAc. The organic layers were concentrated under vacuum. The residue was purified with 1-5% MeOH/DCM to afford Intermediate 14. ¹H NMR (300 MHz, CDCl₃, ppm): 8.90 (s, 1H), 8.24 (d, J=7.8 Hz, 1H), 7.96 (d, J=8.4 Hz, 1H), 7.50-7.37 (m, 3H), 7.36-7.27 (m, 10H), 5.10-5.05 (m, 4H), 4.57-4.50 (m, 1H), 4.34 (s, 3H), 4.25-4.20 (m, 1H), 3.26-3.18 (m, 1H), 3.12-3.03 (m, 1H). MS (m/z): 625 [M+H].

Method B: DBU (20 μl, 0.13 mmol) was added with stirring to the solution of Compound of Example 1 (10 mg, 0.027 mmol) in DMF (200 μl) at r.t. After ca. 5 min, tetrabenzylpyrophosphate (TBPP) was added. The resulting mixture was stirred overnight. TLC (5% MeOH/DCM) indicated ca. 90% completion. The reaction was quenched with 2N HCl aq., extracted with EtOAc, washed with brine, and purified with PTLC to afford Intermediate 14. MS (m/z): 625 [M+H].

Compound of Example 4. Intermediate 14 (20 mg, 0.032 mmol) was dissolved in EtOAc (10 ml) and MeOH (3 ml). Pd/C (ca 10 mg) was added, and the mixture was stirred under H₂ for 3 h. The mixture was filtered through Celite, and concentrated under vacuum to afford the product. ¹H NMR (300 MHz, D₂O, ppm): 8.44 (br, 1H), 7.86-7.72 (m, 2H), 7.32-7.20 (m, 2H), 7.00 (d, J=8.4 Hz, 1H), 4.64-4.51 (m, 1H), 4.31 (s, 3H), 4.19-3.96 (m, 2H), 3.28-3.14 (m, 1H), 3.08-2.96 (m, 1H). MS (m/z): 445 [M+H].

### Example 5

### Compound of Structure

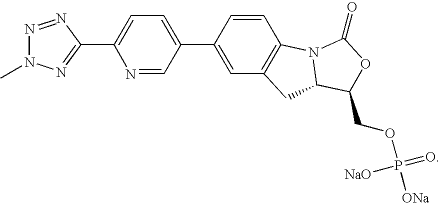

Method A: Scheme for Compound of Example 5:

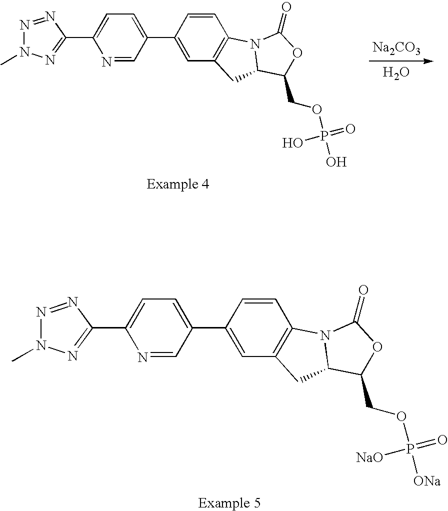

Compound of Example 4 (11.7 mg, 0.026 mmol) was dissolved in water (1-2 ml), and added Na₂CO₃ (2.80 mg, 0.026 mmol). The solution was left at r.t. for 30 min., then filtered and lyophilized to afford the product. ¹H NMR (300 MHz, D₂O, ppm): 8.64 (br, 1H), 8.00-7.91 (m, 2H), 7.45 (s, 1H), 7.40 (d, J=8.4 Hz, 1H), 7.18 (d, J=8.1 Hz, 1H), 4.84-4.76 (m, 1H), 4.52-4.48 (m, 1H), 4.36 (s, 31-1), 4.08-3.94 (m, 2H), 3.34-3.32 (m, 1H), 3.17-3.09 (m, 1H). MS (m/z): 445 [M+H, acid].

Method B: Scheme for Compound of Example 5:

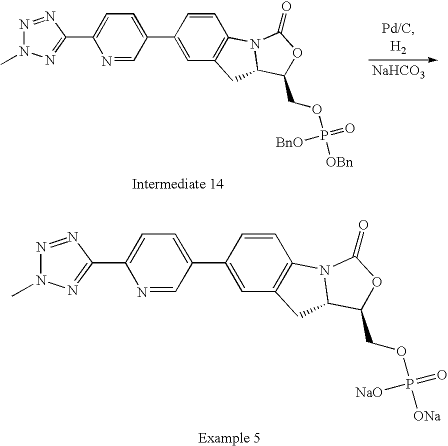

NaHCO₃ (102 mg, 1.22 mmol) in water (10 mL) was added to Pd/C (120 mg) and Intermediate 14 (400 mg, 0.64 mmol) in THF (4 mL). The mixture was hydrogenated for 3 h, then filtered and washed with water. Solvent was removed under vacuum, and the solid was dissolved in water (3 mL). The mixture was filtered, and EtOH (15 mL) was added to the filtrate. Resulted precipitate was filtered off and washed with EtOH and EtOAc, and dried to afford the product as a white solid. MS (m/z): 445 [M-2Na+3H] (in agreement with the ionized diphosphoric acid form).

### Example 6

### Compound of Structure

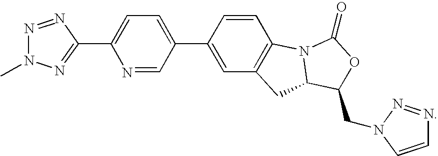

Scheme for Compound of Example 6:

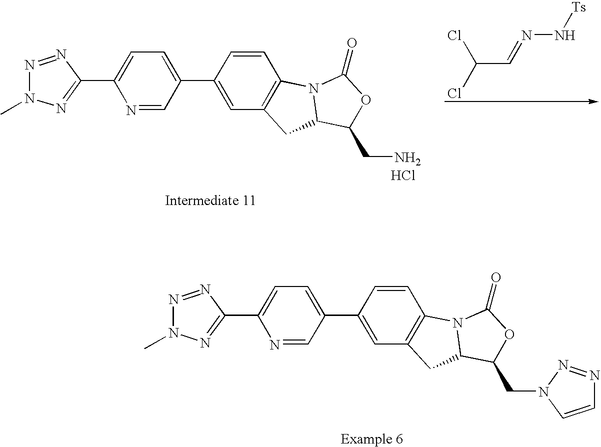

Compound of Example 6. Intermediate 11 (40 mg, 0.11 mmol) was dissolved in MeOH 5 ml, and triethylamine (0.15 ml, 1.07 mmol). After cooled to 0° C., the solution was added 1-[(2,2-dichloro-ethylidene)]-2-(toluenesulfonyl) hydrazide (34 mg, 0.12 mmol). The reaction was left at r.t. overnight, and then quenched with water, extracted with EtOAc. Solvent was removed under vacuum, and the crude material was purified by PTLC eluting with 5% MeOH/DCM to afford Compound of Example 6. ¹H NMR (300 MHz, CD₃OD, ppm): 8.866 (s, 1H), 8.23 (d, J=8.1 Hz, 1H), 8.10-8.07 (dd, J=8.10 and 2.1 Hz, 1H), 8.01 (d, J=0.6 Hz, 1H), 7.76 (m, 1H), 7.55-7.54 (m, 2H), 7.45-7.42 (m, 1H), 5.10-5.02 (m, 1H), 4.98-4.95 (m, 2H), 4.78-4.71 (m, 1H), 4.46 (s, 3H), 3.46-3.32 (m, 1H), 3.27-3.22 (m, 1H). MS (m/z): 416 [M+H].

### Example 7

### Compound of Structure

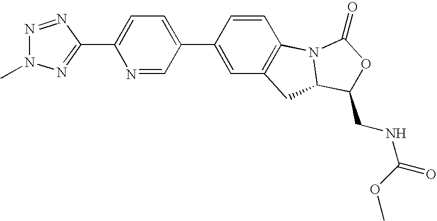

Scheme for Compound of Example 7:

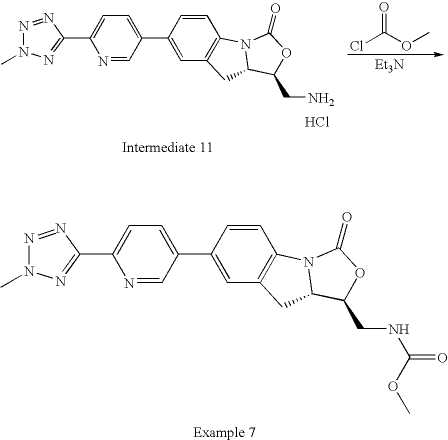

Compound of Example 7. Intermediate 11 (25 mg, 0.069 mmol) and triethylamine (29 μl, 0.21 mmol) were mixed in DCM (ca. 1 mL). Methyl chloroformate (5.9 μl, 0.076 mmol) was added to the solution. The mixture was stirred overnight, and then quenched with EtOAc/water, and extracted with EtOAc. Combined organic layers were concentrated in vacuo. The residue was purified by PTLC to afford Compound of Example 7. ¹H NMR (300 MHz, DMSO-d₆, ppm): 9.00 (t, J=1.2 Hz, 1H), 8.25-8.22 (dd, J=2.4 and 8.4 Hz, 1H), 8.15 (d, J=8.4 Hz, 1H), 7.75 (s, 1H), 7.70 (d, J=8.4 Hz, 1H), 7.59 (t, J=6.0 Hz, 1H), 7.35 (d, J=8.4 Hz, 1H), 4.79-4.68 (m, 1H), 4.62-4.52 (m, 1H), 4.45 (s, 3H), 3.56 (s, 3H), 3.51-3.44 (m, 2H), 3.28-3.22 (m, 2H). MS (m/z): 422 [M+H].

### Example 8

### Compound of Structure

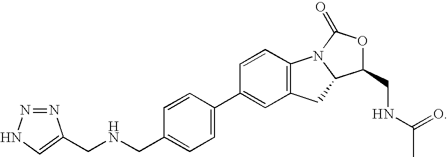

Scheme for Compound of Example 8:

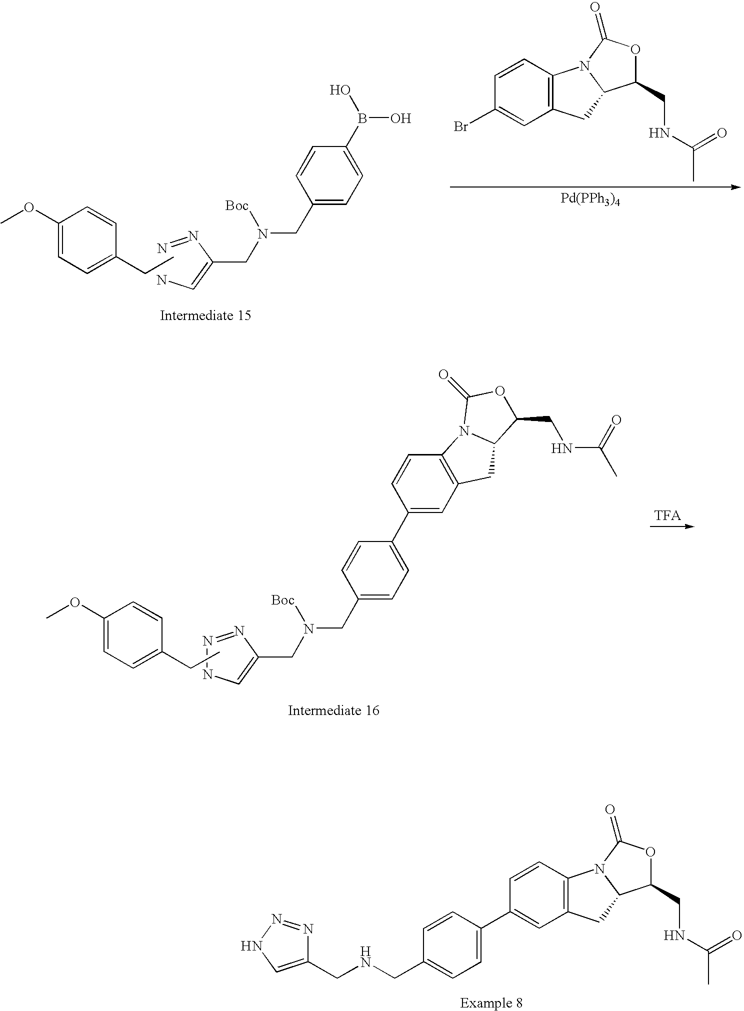

Intermediate 16. A solution of Intermediate 15 (279 mg, 0.62 mmol, prepared according to WO 2006/133397), N-(((1S,9aS)-7-bromo-3-oxo-1,3,9,9a-tetrahydrooxazolo[3,4-a]indol-1-yl)methyl)acetamide (200 mg, 0.62 mmol; prepared as described in PCT WO 91/07409) in 1,4-dioxane (10 mL), after degassed, were added K₂CO₃ (255 mg, 1.85 mmol), and Pd(PPh₃)₄ (71.7 mg, 0.062 mmol). The mixture was flushed with N², and stirred at 78-80° C. overnight. Extra boronic ester (140 mg, 0.31 mmol) and K₂CO₃ (133 mg, 0.93 mmol were added. The mixture was degassed with N2 and stirred at 78-80° C. for 12 h. It was then filtered aiding with EtOAc/DCM. Filtrate was concentrated in vacuo, and the product was purified by column chromatography (2-5% MeOH/DCM). ¹H NMR (300 MHz, CD₃OD-d₄, ppm): 7.58-7.42 (m, 5H), 7.37 (d, J=8.7 Hz, 1H), 7.20 (d, J=8.4 Hz, 2H), 7.15 (d, J=8.4 Hz, 1H), 7.06 (d, J=8.1 Hz, 1H), 6.84 (d, J=8.4 Hz, 1H), 7.37 (d, 1H), 5.47 (s, 3H), 5.43 (s, 1H), 4.74-4.67 (m, 1H), 4.62-4.53 (m, 1H), 4.48 (br.s., 2H), 4.33 (s, 1H), 3.76 (s, 3H), 3.66 (ss, 3H), 3.37-3.28 (m, 1H), 3.24-3.12 (m, 1H), 2.02 (s, 3H); 1.42 (s, 9H). MS (m/z): 653 [M+H].

Compound of Example 8. Intermediate 16 (200 mg, 0.31 mmol) was dissolved in TFA (4 ml), and the mixture was stirred at r.t. overnight, then heated at 55° C. for 1 h. TFA was removed under vacuum, and the residue was redissolved in EtOAc, neutralized with saturated aq. NaHCO₃, and extracted with EtOAc. Combined organic layers were dried (MgSO₄) and concentrated in vacuo. The product was purified by PTLC (0.5-1% triethyl amine in 4-7% MeOH/DCM). ¹H NMR (300 MHz, CD₃OD, ppm): 7.94 (s, br, 1H), 7.71-7.68 (m, 2H), 7.56-7.52 (m, 4H), 7.39 (d, J=8.40 Hz, 1H), 4.74-4.68 (m, 1H), 4.63-4.54 (m, 1H), 4.42 (s, 2H), 4.33 (s, 2H), 3.67 (d, J=4.80 Hz, 2H), 3.40-3.31 (m, 1H), 3.27-3.23 (m, 2H), 2.02 (s, 3H). MS (m/z): 433 [M+14].

### Example 9

### Compound of Structure

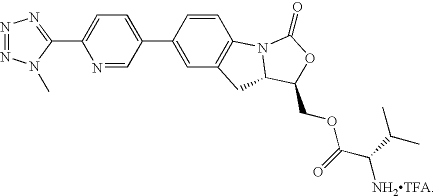

Scheme for Compound of Example 9:

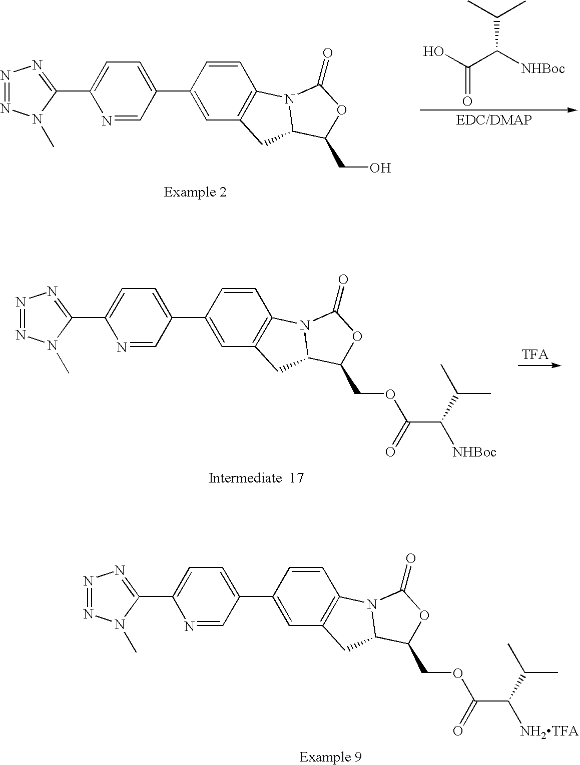

Intermediate 17. The solution of Compound of Example 2 (60 mg, 0.0027 mmol) in DMF (ca. 0.5 mL) is added to a premixed solution containing Boc-L-valine (0.0027 mmol), N-ethyl-N′-dimethylaminopropyl carbodiimide (EDC; 0.0027 mmol), and dimethylaminopyridine (DMAP; 0.0027 mmol) in DMF at r.t. The mixture is stirred overnight. It is then quenched with water and extracted with EtOAc. After removing the solvent, the residue is purified by PTLC to obtain the product.

Compound of Example 9. Intermediate 17 is treated with 20% TFA/DCM for 2 h at r.t. The reaction is concentrated in vacuo, and recrystallized with EtOAc/Hexane to afford the compound of Example 9.

### Example 10

### Compound of Structure

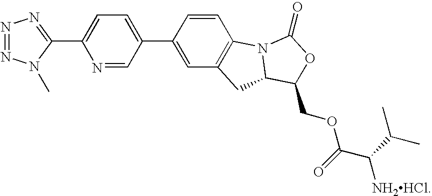

Scheme for Compound of Example 10:

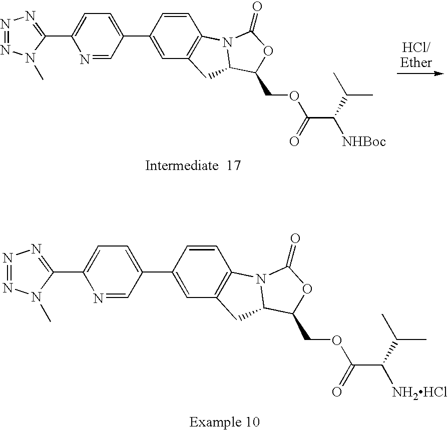

Compound of Example 10. This compound is prepared according to a procedure in the last step for preparation of Compound of Example 9, except using 2N HCl in ether instead of 20% TFA/DCM.

### Example 11

### Compound of Structure

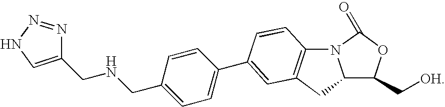

Scheme for Compound of Example 11:

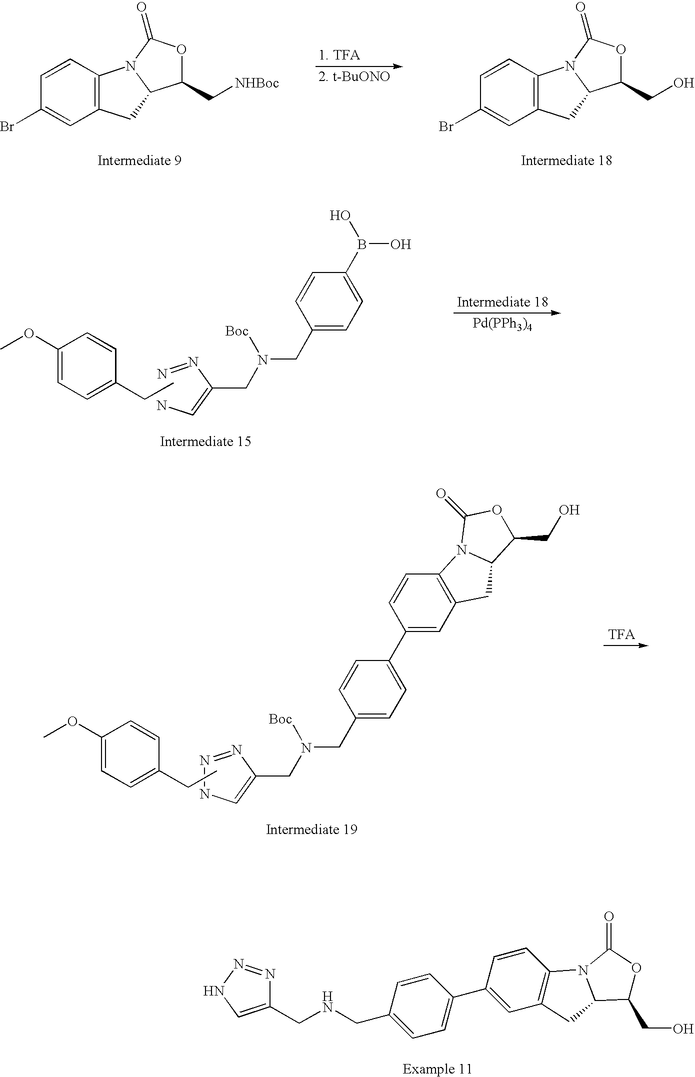

Intermediate 18. TFA (56.6 mL) was added slowly to Intermediate 9 (35.2 g, 91.8 mmol) in dichloroethane (DCE; 153 mL) at 0° C., and the mixture was stirred at r.t. for 1 h. Solvent was removed under vacuum, then DCE (ca. 50 mL) was added, and the mixture was evaporated under vacuum. The latter process was repeated 4 times, and the residue was dried under high vacuum for more than 24 h to afford the amine TFA salt as a brownish foaming solid (37.6 g, 100%).

A 250 mL flask was charged with the above TFA salt (ca. 2.46 g, 6 mmol), PhOH (0.565 g, 6 mmol) and 1,4-dioxane (43 mL) under Ar. TFA (0.924 mL, 12 mmol) was added slowly at 0° C., followed by t-BuONO (6.5 mL, 54 mmol). The reaction mixture was allowed to warm up to r.t. and stirred for 3 h. The reaction mixture was cooled to 0° C., and saturated aq. NaHCO₃ (ca. 19 mL) was added slowly to adjust pH to ca. 8. The mixture was stirred for 30 min and then extracted with EtOAc (76 mL×3). The combined EtOAc layers were dried over anhydrous Na₂SO₄, filtered and condensed under vacuum. The residue was purified by silica gel column using 100% petroleum ether as initial eluent followed with petroleum ether-EtOAc (5:1 to 1:1). The product was obtained as a white solid (1.2 g, 71%). ¹H-NMR (400 MHz, CDCl₃, ppm); δ 7.37 (m, 3H); 4.75 (m, 1H); 4.60 (m, 1H); 4.06 (m, 1H); 3.88 (m, 1H); 3.30 (m, 1H); 3.15 (m, 1H).

Intermediate 19. This intermediate was made according to the same procedure just as described for the preparation of Intermediate 16, except using Intermediate 18 instead of N-(((1S,9aS)-7-bromo-3-oxo-1,3,9,9a-tetrahydrooxazolo[3,4-a]indol-1-yl)methyl)acetamide.

Compound of Example 11. This compound was prepared according to a procedure for preparation of Compound of Example 8, except using Intermediate 19 instead of Intermediate 16. ¹H NMR (300 MHz, CD₃OD/CDCl₃, ppm): 7.95 (s, 1H), 7.84 (s, 1H), 7.81-7.70 (m, 2H), 7.68-7.60 (m, 4H), 4.98-4.93 (m, 1H), 4.74-4.72 (m, 1H), 4.22 (s, 2H), 4.21-4.15 (m, 1H), 4.12 (s, 2H), 3.79-3.60 (m, 2H), 3.38-3.33 (m, 1H). MS (m/z): 392 [M+H].

### Example 12

### Compound of Structure

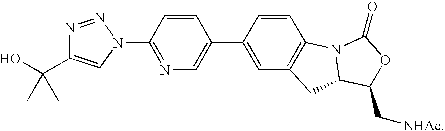

Scheme for Compound of Example 12:

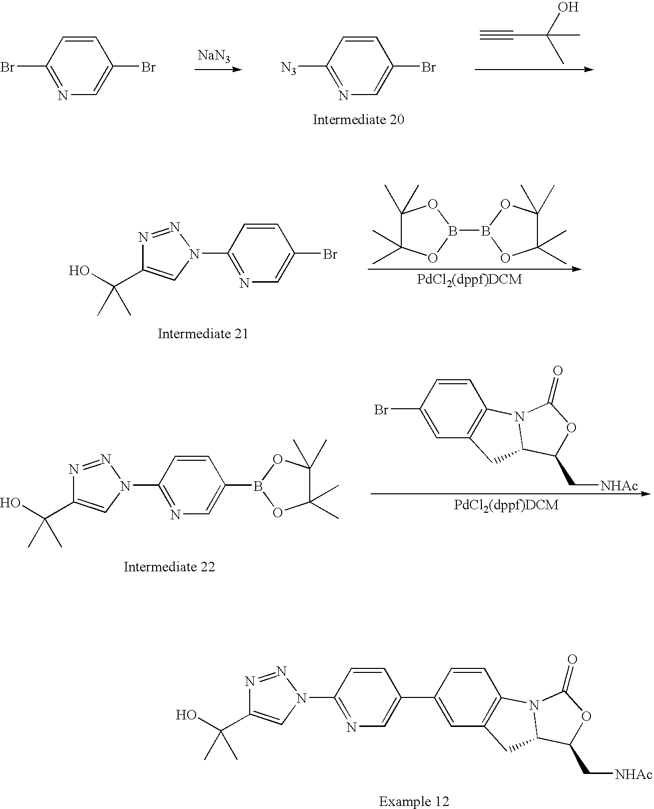

Intermediate 20. Sodium azide (15.0 g, 230.7 mmol) was added to a solution of 2,5-dibromopyridine (12.0 g, 50.6 mmol) DMF (50 mL). The reaction mixture was heated to 90° C. and stirred overnight. After cooling to r.t., the reaction mixture was poured into water (200 mL) and extracted with petroleum ether (200 mL). The aqueous layer was collected and extracted with ethyl acetate (150 mL×3). The organic layers were combined, washed with brine and dried (Na₂SO₄). Solvent was evaporated under vacuum and the product was obtained as a yellowish solid.

Intermediate 21. A 50 mL flask was charged with the Intermediate 20 (500 mg, 2.5 mmol), 1,1-dimethyl propargyl alcohol (370 uL, 3.8 mmol), sodium ascorbate (500 mg, 2.5 mmol) and CuSO₄.5H₂O (150 mg, 0.6 mmol). A mixed solvent of water (10 mL) and alcohol (10 mL) was added. The reaction mixture was stirred overnight at r.t. The mixture was condensed under vacuum and the residue was taken into EtOAc (20 mL) and washed with water, brine, dried (Na₂SO₄), and concentrated. The crude product was purified by column chromatograph (30% EtOAc/petroleum ether) and the product was obtained as a solid. ¹H-NMR (400 MHz, CDCl₃, ppm): 8.58 (d, J=2.0 Hz, 1H); 8.46 (s, 1H); 8.14 (d, J=8.8 Hz, 1H); 8.06 (dd, J=8.8, 2.4 Hz, 1H); 2.15 (br, 1H); 1.73 (s, 6H). MS (m/z): 282.8, 284.8 [M+H].

Intermediate 22. PdCl₂(dppf).DCM (99 mg, 0.13 mmol) was added to a suspension of the Intermediate 21 (250 mg, 0.88 mmol), bis(pinacolato)diborane (340 mg, 1.3 mmol) and KOAc (260 mg, 2.6 mmol) in 1,4-dioxane (3.0 mL). The slurry was degassed under Ar for 20 min., then stirred o.n. at 80° C. The reaction mixture was taken into ethyl acetate (20 mL) and filtered through a short Celite pad. The filtrate was washed with water, brine, dried (Na₂SO₄), and concentrated. The product was purified by column chromatograph (30% EtOAc/petroleum ether). ¹H NMR (400 MHz, CDCl₃, ppm): 8.82 (br, 1H); 8.54 (s, 1H); 8.27 (dd, J=8.0, 1.2 Hz, 1H); 8.16 (d, J=8.0 Hz, 1H); 2.32 (br, 1H); 1.72 (s, 6H); 1.39 (s, 12H).

Compound of Example 12. PdCl₂(dppf).DCM (35 mg, 0.044 mmol) was added to a suspension of Intermediate 22 (106 mg, 0.32 mmol), N-(((1S,9aS)-7-bromo-3-oxo-1,3,9,9a-tetrahydrooxazolo[3,4-a]indol-1-yl)methyl)acetamide (95 mg, 0.30 mmol) and K₂CO₃ (120 mg, 0.88 mmol) in 1,4-dioxane (2.0 mL) and water (0.5 mL). The slurry was degassed under Ar for 20 minutes, then stirred o.n. at 80° C. The reaction mixture was taken into ethyl acetate (20 mL) and filtered through a short Celite pad. The filtrate was washed with water, brine, dried (Na₂SO₄), then filtered and concentrated. The product was purified by column chromatograph (4% MeOH/DCM). ¹H-NMR (400 MHz, DMSO-d₆, ppm): 8.88 (d, J=2.0 Hz, 1H); 8.57 (s, 1H); 8.37 (dd, J=8.8, 2.0 Hz, 1H); 8.31 (t, J=7.2 Hz, 1H); 8.17 (d, J=8.0 Hz, 1H); 7.77 (s, 1H); 7.73 (d, J=8.4 Hz, 1H); 7.39 (d, J=8.0 Hz, 1H); 5.30 (s, 1H); 4.77 (m, 1H); 4.58 (m, 1H); 3.55 (t, J=5.6 Hz, 2H); 3.30 (m, overlapped with DMSO-d₆, 2H); 1.89 (s, 3H); 1.55 (s, 6H). MS (m/z): 449 [M+H].

### Example 13

### Compound of Structure

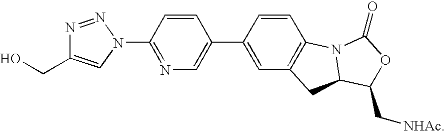

Scheme for Compound of Example 13:

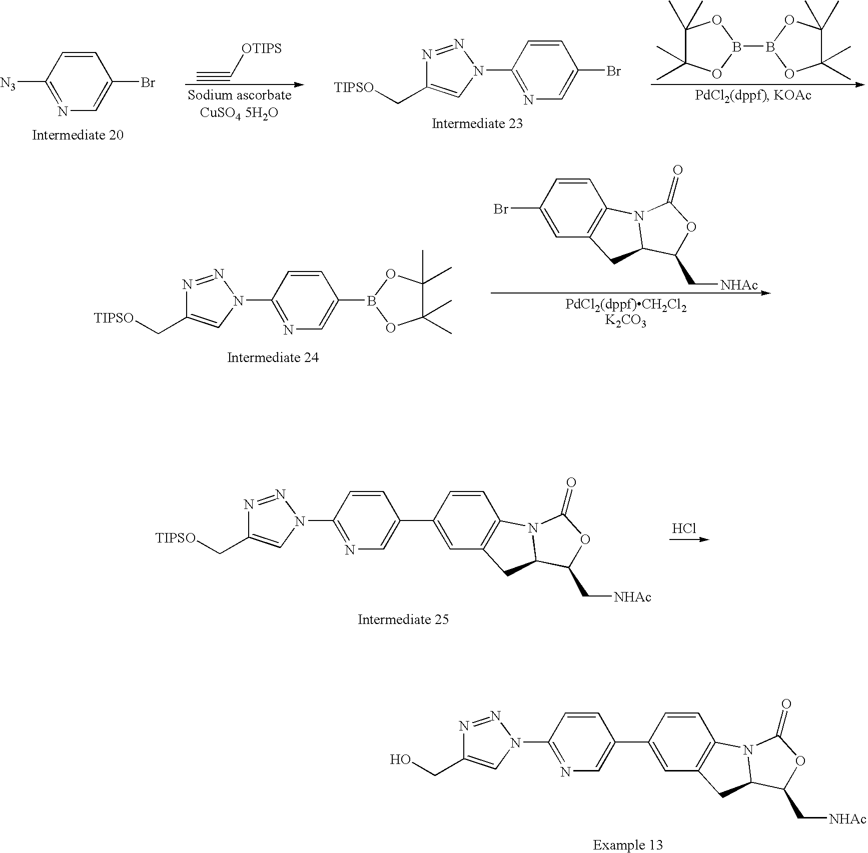

Intermediate 23. 2-Azido-5-bromopyridine (Intermediate 20; 700 mg, 3.5 mmol) and triisopropylsilyl propargyl ether (800 mg, 3.2 mmol) were dissolved in 30 mL of ethanol and 20 mL of water. Sodium ascorbate (200 mg, 0.9 mmol) was added to the mixture, followed by CuSO₄.5H₂O (80 mg, 1.6 mmol). The mixture was stirred at r.t. over the weekend. Solvent was removed under vacuum and the residue was extracted with EtOAc (60 mL×3). The combined organic layers were washed with brine (60 mL), dried (Na₂SO₄) and filtered. The filtrate was condensed and the residue was further purified by passing through a short silica gel column and washed with a solution of 10% EtOAc in petroleum ether. The filtrate was condensed and the product was obtained as oil.

Intermediate 24. Intermediate 23 (400 mg, 0.1 mmol) was dissolved in 8 mL of anhydrous 1,4-dioxane. Bis(pinacolato)diborane (600 mg, 2.4 mmol) was added, followed by KOAc (400 mg, 4.0 mmol) and PdCl₂(dppf).CH₂Cl₂ (75 mg, 1.0 mmol). The reaction mixture was degassed for half an hour, and then heated to 80° C. and stirred overnight. The dark solution was filtered through Celite and washed with 100 mL of ethyl acetate. The filtrate was concentrated and washed with 10% NH₄Cl, brine, and dried (Na₂SO₄). Solvent was removed under vacuum and the residue was dissolved in ether and filtered through a short silica gel pad. The filtrate was concentrated and the formed solid was washed with methanol. The product was obtained as a white solid.

Intermediate 25. N-4(1S,9aS)-7-bromo-3-oxo-1,3,9,9a-tetrahydrooxazolo[3,4-a]indol-1-yl)methyl)acetamide (100 mg, 0.31 mmol) was dissolved in a mixed solvent of dioxane (4 mL), ethanol (1 mL) and water (1 mL). Intermediate 24 (160 mg, 0.35 mmol) was added, followed by PdCl₂(dppf).CH₂Cl₂ (30 mg, 0.04 mmol) and K₂CO₃ (140 mg, 1.0 mmol). The reaction mixture was degassed for half an hour, and then heated to 80° C. and stirred overnight. The reaction mixture was filtered through Celite and washed with 50 mL of ethyl acetate. The filtrate was concentrated and washed with 10% NH₄Cl, brine, and dried (Na₂SO₄). Solvent was removed under vacuum. The residue was purified by preparative TLC (5% MeOH/DCM) and the product was obtained as a white solid.

Compound of Example 13. Intermediate 24 (40 mg, 0.069 mmol) was dissolved in a mixed solvent of DCM (2 mL) and MeOH (2 mL). A solution of HCl in isopropyl ether (4.0 M, 6 mL) was added and the mixture was stirred for 3 hours at r.t. Solvent was removed under vacuum and the residue was purified by preparative TLC (5% MeOH/DCM). The product was obtained as a white solid. NMR (400 MHz, DMSO-d₆, ppm): 8.89 (d, J=2.0 Hz, 1H); 8.70 (s, 1H); 8.37 (m, 2H); 8.18 (d, J=8.4 Hz, 1H); 7.78 (s, 1H); 7.72 (d, J=8.4 Hz, 1H); 7.38 (d, J=8.0 Hz, 1H); 5.36 (t, J=6.0 Hz, 1H); 4.77 (m, 1H); 4.64 (d, J=6.0 Hz, 1H); 4.56 (m, 1H); 3.55 (t, J=7.4 Hz, 1H); 3.31 (m, 1H); 1.88 (d, J=4.8 Hz, 3H). MS (m/z): 421 [M+H].

### Example 14

### Compound of Structure

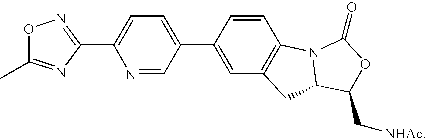

Scheme for Compound of Example 14:

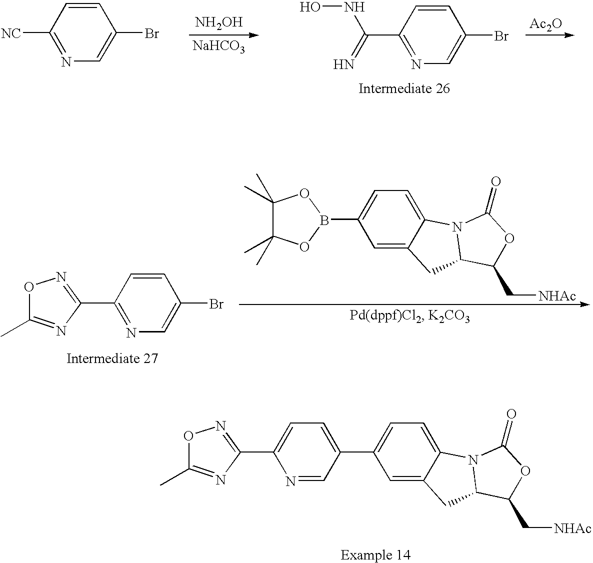

Intermediate 26. 5-Bromopicolinonitrile (1.5 g, 8.2 mmol) was dissolved in 15 mL of EtOH. To this solution was added HCl salt of hydroxylamine (2.9 g, 41.7 mmol), followed by NaHCO₃ (3.5 g, 41.7 mmol). The reaction mixture was heated to reflux for 2 hrs. After cooling to r.t., most of the solvent was removed. Water was added and the mixture was extracted with EtOAc (50 mL×2). The combined organic layers were washed with brine and dried (Na₂SO₄). Solvent was removed and the resulting white solid was used directly for the next step without further purification.

Intermediate 27. Intermediate 26 (1.8 g, 8.3 mmol) was dissolved in 50 mL of acetic anhydride and the mixture was heated to reflux for 2 hrs. The solution turned from clear to dark brown. After cooling to r.t., the reaction mixture was put on pump to remove most of the solvent. The residue was purified by column chromatography (33% EtOAc/petroleum ether) and the product was obtained as a yellow solid.

Compound of Example 14. Intermediate 27 (72 mg, 0.30 mmol) was dissolved in a mixture of dioxane (4 mL), water (1 mL) and ethanol (1 mL). To this mixture was added N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide (75 mg, 0.20 mmol), followed by PdCl₂(dppf).CH₂Cl₂ (23 mg, 0.03 mmol) and K₂CO₃ (83 mg, 0.60 mmol). The reaction mixture was degassed for 30 min before heated to 75° C. After heating overnight, the mixture was passed through a short Celite pad. The filtrate was concentrated and purified by column chromatography (5% MeOH/DCM). The product was obtained as a white solid. ¹H-NMR (400 MHz, DMSO-d₆, ppm): 9.06 (d, J=2.0 Hz, 1H); 8.33 (t, J=6.0 Hz, 1H); 8.27 (dd, J=8.0, 2.4 Hz, 1H); 8.12 (d, J=8.0 Hz, 1H); 7.79 (s, 1H); 7.73 (d, J=8.0 Hz, 1H); 7.38 (d, J=8.0 Hz, 1H); 4.77 (m, 1H); 4.57 (q, J=8.8 Hz, 1H); 3.55 (t, J=5.2 Hz, 1H); 2.71 (s, 3H); 1.89 (s, 3H). MS (m/z): 406 [M+H].

### Example 15

### Compound of Structure

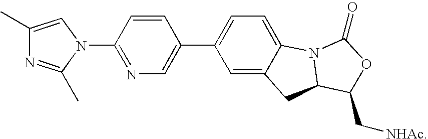

Scheme for Compound of Example 15:

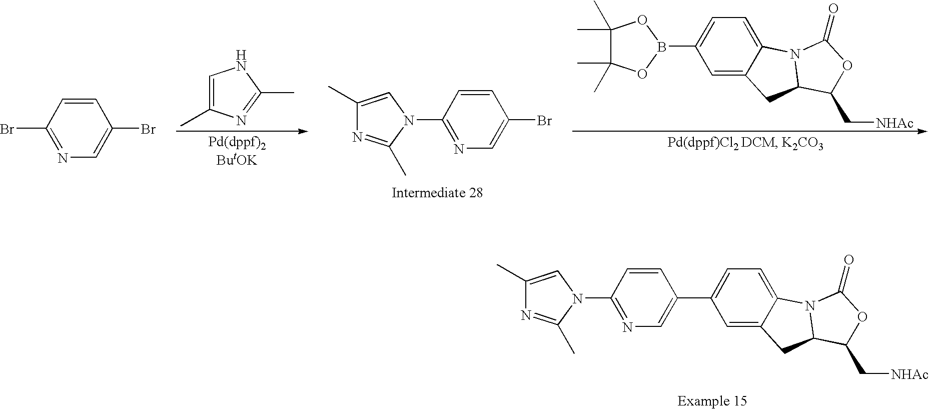

Intermediate 28. 2,5-Dibromopyridine (1.2 g; 5.0 mmol) and 2,4-dimethyl-1H-imidazole (0.5 g; 5.0 mmol) were dissolved in 10 mL of DMF. PdCl₂(dppf).CH₂Cl₂ (20 mg; 0.025 mmol) was added, followed by Bu^{t}OK (0.56 g; 5.0 mmol). The reaction mixture was degassed under Ar for 30 min. and then heated at 100° C. overnight. After cooling to r.t., the mixture was filtered through Celite and washed with EtOAc (50 mL). The filtrate was washed with 10% NH₄Cl and brine, dried (Na₂SO₄) and concentrated. The residue was purified by preparative TLC (3% MeOH/DCM) and the product was obtained as a white solid. ¹H NMR (400 MHz, CDCl₃, ppm): 8.62 (d, J=2.0 Hz, 1H); 7.97 (dd, J=8.0, 2.0 Hz, 1H); 7.23 (d, J=8.0 Hz, 1H); 7.01 (s, 1H); 2.65 (s, 3H); 2.30 (s, 3H). MS (m/z): 254 [M+H].

Compound of Example 15. Intermediate 28 (79 mg, 0.31 mmol) was dissolved in a mixed solvent of dioxane (5 mL) and water (1 mL). N-(41S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide (116 mg, 0.31 mmol) was added, followed by PdCl₂(dppf).CH₂Cl₂ (15 mg, 0.031 mmol) and K₂CO₃ (207 mg, 1.50 mmol). The reaction mixture was degassed for 30 min., and then heated to 80° C. and stirred overnight. The reaction mixture was filtered through Celite and washed with 50 mL of ethyl acetate. The filtrate was concentrated and washed with 10% NH₄Cl, brine, and dried (Na₂SO₄). Solvent was removed under vacuum. The residue was purified by preparative TLC (5% methanol/DCM), and the desired product was obtained as a white solid. NMR (400 MHz, CDCl₃, ppm): 8.73 (d, J=2.0 Hz, 1H); 7.96-8.01 (dd, J=12.0, 2.0 Hz, 1H); 7.47-7.59 (m, 3H); 7.37 (d, J=12.0 Hz, 1H); 7.06 (s, 1H); 6.04 (br, 1H); 4.60 (m, 2H); 3.84 (m, 1H); 3.74 (m, 1H); 3.40 (m, 1H); 3.24 (m, 1H); 2.70 (s, 3H); 2.32 (s, 3H), 2.10 (s, 3H). MS (m/z): 418 [M+H].

### Example 16

### Compound of Structure

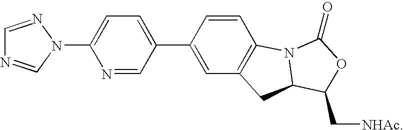

Scheme for Compound of Example 16:

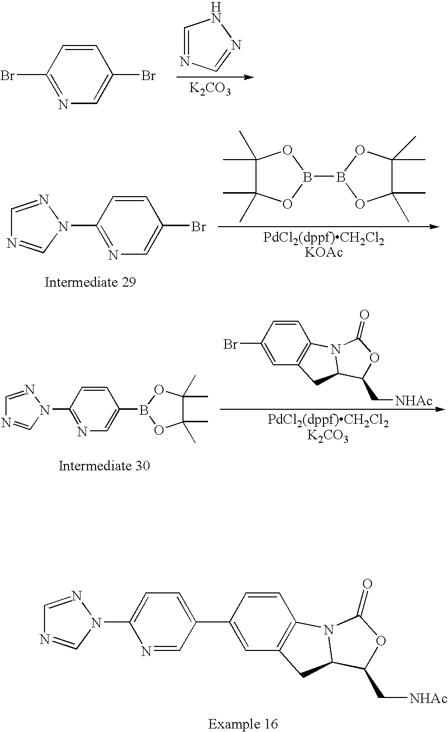

Intermediate 29. 2,5-Dibromopyridine (1.0 g, 4.2 mmol) and 1H-1,2,4-triazole (0.35 g, 5.0 mmol) were dissolved in 10 mL of NMP. Solid K₂CO₃ (1.7 g, 12.5 mmol) was added and the reaction mixture was heated to 100° C. and stirred overnight. TLC showed 2,5-dibromopyridine disappeared. After cooling to r.t., the reaction mixture was poured into water (50 mL) and the product filtered and dried under vacuum. MS (m/z): 227 [M+H].

Intermediate 30. PdCl₂(dppf).CH₂Cl₂ (50 mg, 0.065 mmol) was added to a suspension of Intermediate 29 (300 mg, 1.3 mmol), bis(pinacolato)diborane (518 mg, 2.0 mmol) and KOAc (400 mg, 4.0 mmol) in DMF (2 mL). The slurry was degassed with Ar for 20 min., then stirred overnight at 80° C. After cooling to r.t., the reaction mixture was taken into EtOAc (20 mL) and filtered through a short Celite pad. The filtrate was washed with water, brine, dried (Na₂SO₄) and concentrated. The residue was purified by column chromatograph (30% EtOAc/petroleum ether) and the product was obtained as a white solid. ¹H NMR (400 MHz, CDCl₃, ppm): 9.28 (s, 1H); 8.80 (s, 1H); 8.27 (dd, J=8.0, 1.6 Hz, 1H); 8.14 (s, 1H); 7.92 (d, J=8.0 Hz, 1H); 1.39 (s, 12H). MS (m/z): 273 [M+H].

Compound of Example 16. PdCl₂(dppf).CH₂Cl₂ (35 mg, 0.044 mmol) was added to a suspension of Intermediate 30 (124 mg, 0.45 mmol), N-(((1S,9aS)-7-bromo-3-oxo-1,3,9,9a-tetrahydrooxazolo[3,4-a]indol-1-yl)methyl)acetamide (100 mg, 0.30 mmol) and K₂CO₃ (128 mg, 0.90 mmol) in dioxane (2.0 mL) and water (0.5 mL). The slurry was degassed with Ar for 20 min., then stirred overnight at 80° C. After cooling down to r.t., the reaction mixture was taken into ethyl acetate (20 mL) and filtered through a short Celite pad. The filtrate was washed with water, brine, dried (Na₂SO₄) and concentrated. The residue was purified by column chromatograph (4% MeOH/DCM) and the product was obtained as a white solid. ¹H NMR (400 MHz, DMSO-d₆, ppm): 9.42 (s, 1H); 8.83 (s, 1H); 8.34 (br, 3H); 7.95 (d, J=8.4 Hz, 1H); 7.75 (s, 1H); 7.71 (d, J=8.4 Hz, 1H); 7.39 (d, J=8.0 Hz, 1H), 4.78 (m, 1H); 4.55 (m, 1H); 3.54 (t, J=6.0 Hz, 2H); 3.30 (m, overlapped with DMSO-d₆, 2H); 1.89 (s, 3H). MS (m/z): 391 [M+H].

### Example 17

### Compound of Structure

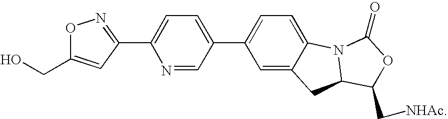

Scheme for Compound of Example 17:

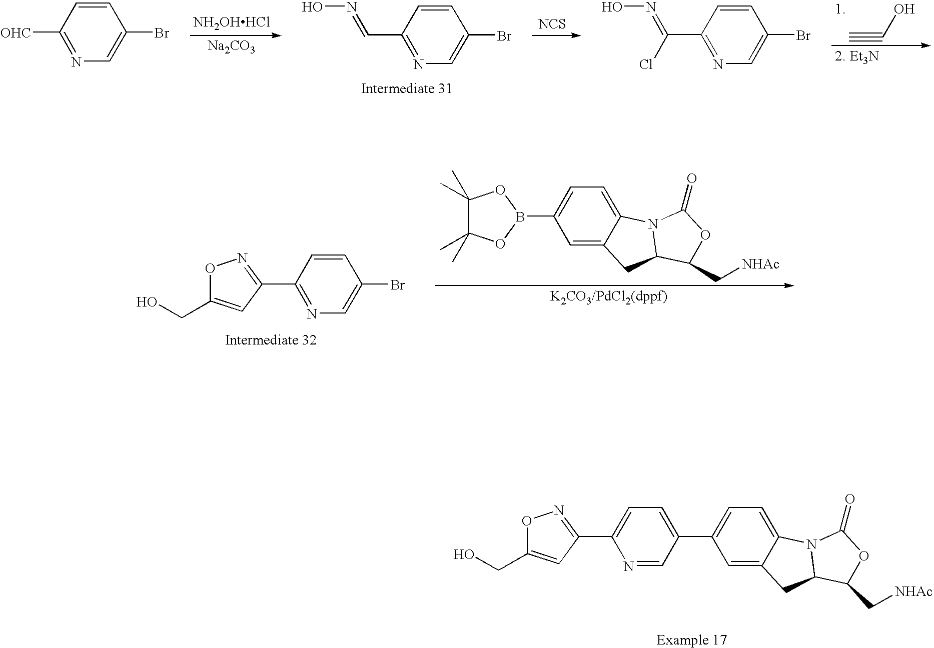

Intermediate 31. 5-Bromopyridine-2-carboxaldehyde (200 mg, 1.08 mmol) was dissolved in a mixed solvent of MeOH (13 mL) and water (8 mL). Na₂CO₃ (285 mg, 2.69 mmol) was added, followed by NH₂OH.HCl (113 mg, 1.62 mmol). The reaction mixture was stirred at room temperature for 30 min. Water (60 mL) was added and the mixture was filtered and washed with water. The collected solid was dried under vacuum. The white solid thus obtained (170 mg, 79%) was used for the next step without further purification.

Intermediate 32. To a solution of Intermediate 31 (100 mg, 0.5 mmol) in DMF (2.5 ml) was added N-chlorosuccinimide (NCS; 80 mg, 0.6 mmol) at r.t. The reaction mixture was heated to 60° C. and stirred for 30 min. before cooling down to 0° C. Prop-2-yne-1-ol (140 mg, 2.5 mmol) was added to the mixture and stirred for 10 min. A mixture of Et₃N in 1 mL of DMF was added and the mixture was stirred for another 30 min. at 0° C. The reaction mixture was warmed up to r.t. and stirred for 1 h. The reaction mixture was then poured into 10 mL of ice water and extracted with EtOAc (40 mL×3). The combined organic layers were washed with brine, dried (Na₂SO₄) and concentrated. The residue was recrystallized from EtOAc and petroleum ether and the product was obtained as a white solid.

Compound of Example 17. 3-(5-Bromopyridin-2-yl)isoxazol-5-yl)methanol (100 mg, 0.39 mmol) was dissolved in a mixed solvent of dioxane (4 mL), ethanol (1 mL) and water (1 mL). N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide (145 mg, 0.39 mmol) was added, followed by PdCl₂(dppf).CH₂Cl₂ (30 mg, 0.040 mmol) and K₂CO₃ (162 mg, 1.17 mmol). The reaction mixture was degassed for 30 min. and stirred overnight at 80° C. The reaction mixture was filtered through Celite and washed with 50 mL of EtOAc. The filtrate was concentrated and washed with 10% NH₄Cl, brine, and dried (Na₂SO₄). Solvent was removed under vacuum. The residue was purified by preparative TLC (5% MeOH/DCM), and the desired product was obtained as a white solid. ¹H NMR (400 MHz, CDCl₃, ppm): 8.85 (s, 1H); 8.12 (d, J=8.0 Hz, 1H); 7.95 (m, 1H); 7.54 (t, J=9.6 Hz, 2H); 7.46 (s, 1H); 6.96 (s, 1H); 6.06 (m, 1H); 4.89 (s, 2H); 4.68 (s, 1H); 4.58 (m, 1H); 3.87 (m, 1H); 3.74 (m, 1H); 3.41 (m, 1H); 3.22 (m, 1H); 2.49 (s, 1H); 2.09 (s, 3H). MS (m/z): 421 [M+H].

### Example 18

### Compound of Structure

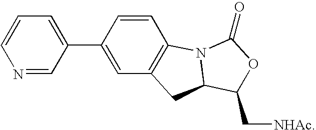

Scheme for Compound of Example 18:

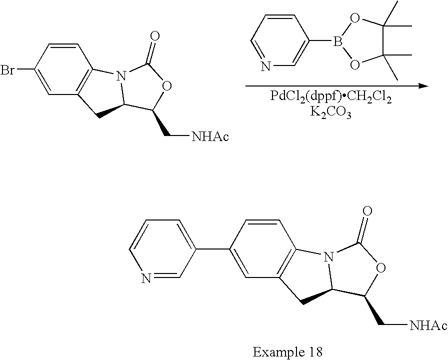

Compound of Example 18. The Compound of Example 18 was prepared as described for the last step in the preparation of Compound of Example 16, except that the Intermediate 30 was substituted for 3-(4,4,5,5-tetramethyl-1,3,2-dioxaborolan-2-yl)pyridine. ¹H NMR (300 MHz, CDCl₃, ppm): 8.79 (d, J=1.8 Hz, 1H), 8.59 (m, 1H); 7.80 (m, 1H), 7.54-7.40 (m, 3H), 7.53 (m, 1H), 6.0 (br. t, 1H), 4.70-4.50 (m, 2H), 3.78-3.64 (m, 2H), 3.43-3.12 (m, 2H), 2.08 (s, 3H). MS (m/z): 324 [M+H].

An exemplary approach to synthesis of the compound of this example is described in PCT WO 91/07409, the contents of which are hereby incorporated by reference herein.

### Example 19

### Compound of Structure

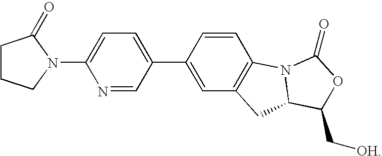

Scheme for Compound of Example 19:

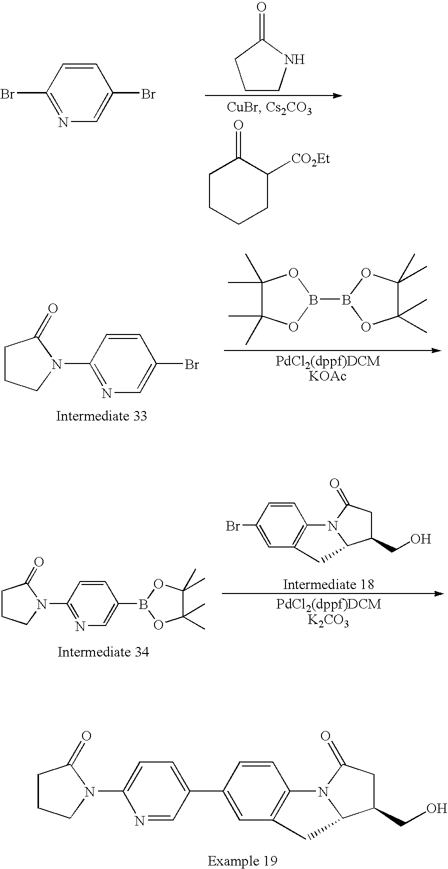

Intermediate 33. CuBr (143 mg, 1.0 mmol), ethyl 2-oxocyclohexanecarboxylate (320 μL, 2.0 mmol) and Cs₂CO₃ (6.84 g, 21 mmol) were placed in a 50 mL flask and purged with N2. DMSO (5 mL) was added and the mixture was stirred for 30 min at r.t. A solution of 2,5-dibromopyridine (2.37 g, 10 mmol) and 2-pyrrolidinone (1.02 g, 12 mmol) in DMSO (5 mL) was added and the mixture was heated at 90° C. overnight. The reaction was quenched with sat. NH₄Cl solution and extracted with EtOAc. The organic layers were combined and washed with brine, dried (Na₂SO₄) and concentrated. The residue was purified by column (EtOAc/petroleum ether=1:6 to 1:3). The desired product was obtained as a pale yellow solid (214 mg).

Intermediate 34. Intermediate 33 (214 mg, 0.89 mmol) was placed in a 10 mL flask. Bis(pinacolato)diborane (451 mg, 2.0 eq.) was added, followed by KOAc (261 mg, 3.0 eq.) and the catalyst (67 mg, 0.1 eq.). The flask was purged with N2 and DMSO (3 mL) was added. The mixture was degassed for 1 h before heating up to 80° C. and keeping this temperature o.n. The mixture was diluted with EtOAc and passed through a short Celite pad. The Celite was washed with EtOAc, and the filtrate was concentrated and washed with brine. The combined organic layers were dried (Na₂SO₄) and concentrated. The residue was purified by column (EtOAc/petroleum ether=1:3 to 1:1). The desired product (Intermediate 34) was obtained as a white solid (120 mg, 47%). ¹HNMR (400 MHz, DMSO-d₆, ppm): 8.57 (d, J=0.8 Hz, 1H); 8.33 (dd, J=8.4, 0.8 Hz, 1H); 8.00 (dd, J=8.4, 1.6 Hz, 1H); 4.00 (t, J=7.2 Hz, 2H); 2.60 (t, J=8.0 Hz, 2H); 2.05 (m, 2H), 1.31 (m, 12H).

Compound of Example 19. Intermediate 34 (120 mg, 0.42 mmol) was mixed with Intermediate 18 (142 mg, 1.2 eq.), K₂CO₃ (115 mg, 2.0 eq.) and PdCl₂(dppf)DCM (34 mg, 0.1 eq.). The flask was purged with N², and 6 mL of 1,4-dioxane was added. The mixture was degassed for 1 h at r.t. and then heated at 70° C. o.n. The mixture was passed through a Celite pad and washed with EtOAc. The filtrate was concentrated and washed with brine. The combined organic layers were dried (Na₂SO₄) and concentrated. The residue was purified by column (EtOAc/petroleum ether=3:1 to 100% EtOAc). The desired product was obtained as a solid (50 mg, 33%). ¹H NMR (400 MHz, DMSO-d₆, ppm): δ 8.67 (d, J=2.0 Hz, 1H); 8.37 (d, J=8.8 Hz, 1H); 8.09 (dd, J=8.8, 2.8 Hz, 1H); 7.65 (s, 1H); 7.60 (d, J=8.0 Hz, 1H); 7.34 (d, J=8.0 Hz, 1H); 5.29 (s, 1H); 4.72 (m, 1H); 4.65 (dd, J=16.4, 9.2 Hz, 1H); 4.04 (t, J=7.2 Hz, 2H); 3.74 (m, 2H); 3.28 (m, 2H); 2.61 (t, J=8.0 Hz, 2H); 2.07 (m, 2H). MS (m/z): 366 [M+H].

### Example 20

### Compound of Structure

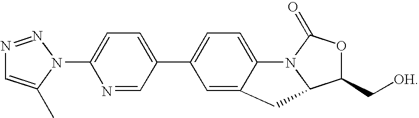

Scheme for Compound of Example 20:

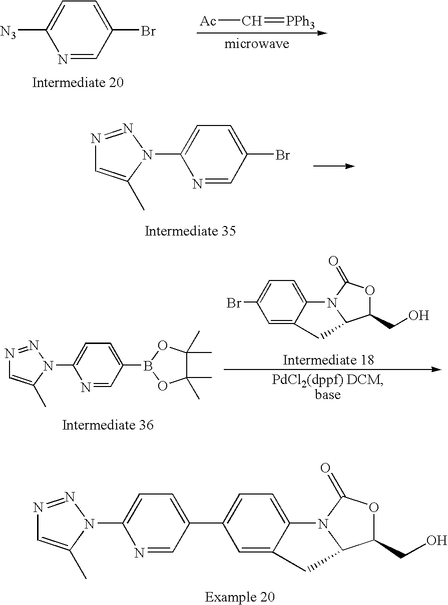

Intermediate 35. A mixture of 2-azido-5-bromopyridine (Intermediate 20) (1.0 g, 5.0 mmol), triphenylacetomethylenephosphine (1.6 g, 5.0 mmol) and silica gel (10 g, 100-200 mesh) was introduced into a microwave oven in an open container. The reaction mixture was irradiated for 4 min and then purified directly by column (EtOAc/Petroleum Ether=1:10). The product was obtained as a solid (0.8 g, 80%).

Intermediate 36. Intermediate 35 (500 mg, 2.1 mmol) was placed in a 10 mL flask. Bis(pinacolato)diborane (800 mg, 1.5 eq.) was added, followed by KOAc (620 mg, 3.0 eq.) and the catalyst (230 mg, 0.15 eq.). The flask was purged with N2 and 1,4-dioxane (6 mL) was added. The mixture was degassed for 1 h before heating up to 75° C. and keeping this temperature o.n. The mixture was diluted with EtOAc and passed through a short Celite pad. The Celite was washed with EtOAc, and the filtrate was concentrated and washed with brine. The combined organic layers were dried (Na₂SO₄) and concentrated. The residue was purified by column (EtOAc/petroleum ether=1:4). The desired product was obtained as a white solid (300 mg, 50%).

Compound of Example 20. Intermediate 36 (106 mg, 0.37 mmol) was mixed with Intermediate 18 (90 mg, 0.31 mmol), K₂CO₃ (100 mg, 2.2 eq.) and the catalyst (40 mg, 0.15 eq.). The flask was purged with N², and 3 mL of 1,4-dioxane was added. The mixture was degassed for 1 h at r.t. and then heated at 80° C. o.n. The mixture was passed through a Celite pad and washed with EtOAc. The filtrate was concentrated and washed with brine. The combined organic layers were dried (Na₂SO₄) and concentrated. The residue was purified by preparative TLC (EtOAc/petroleum ether 3:1). The desired product was obtained as a solid (27 mg, 20%). ¹H NMR (400 MHz, DMSO-d₆, ppm): δ 8.91 (d, J=2.0 Hz, 1H); 8.36 (dd, J=2.0, 8.8 Hz, 1H); 8.00 (d, J=8.0 Hz, 1H); 7.78 (s, 1H); 7.75 (s, 1H); 7.71 (d, J=8.8 Hz, 1H); 7.38 (d, J=8.0 Hz, 1H); 5.30 (t, J=6.0 Hz, 1H); 4.74 (m, 1H); 4.67 (m, 1H); 3.71 (m, 2H); 3.29 (d, J=8.4 Hz, 1H); 2.59 (s 3H). MS (m/z): 364 [M+H].

### Example 21

### Compound of Structure

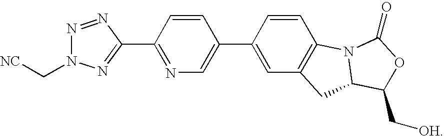

Scheme for Compound of Example 21:

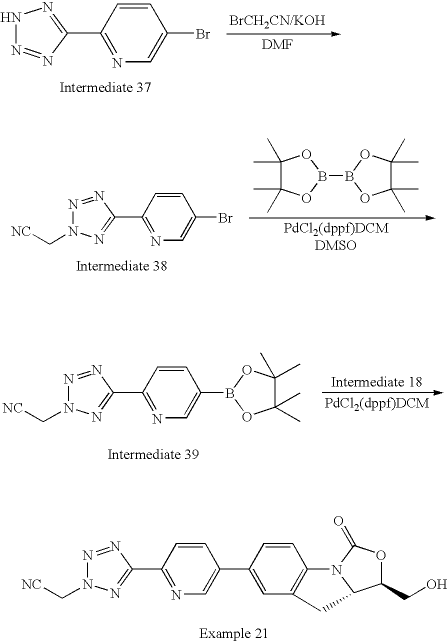

Intermediate 38. KOH (1.7 g, 30.6 mmol) was added to a solution of Intermediate 37 (3.46 g, 15.3 mmol) in 35 mL of DMF at 0° C. Then BrCH₂CN (3.2 mL, 45.9 mmol) was added dropwise. The mixture was stirred at r.t. o.n. Then 400 mL of ice-water was added and extracted with EtOAc. The organic phase was washed with brine, dried and evaporated. Purification by silica gel column (petroleum ether/EtOAc=1/3) gave 2.1 g of Intermediate 38 (yield 52%).

Intermediate 39. Intermediate 38 (1.0 g, 3.77 mmol), pinacol diborane (2.0 g, 7.87 mmol), KOAc (1.2 g, 12.2 mmol) and PdCl₂(dppf)DCM (430 mg, 0.57 mmol) were dissolved in 15 mL of DMSO and the mixture was degassed with Ar for 45 min. The mixture was heated at 80° C. o.n. Then 50 mL of H₂O and 40 mL of EtOAc was added to the mixture. The mixture was filtered, extracted with EtOAc and washed with brine. The mixture was purified by silica gel column to give Intermediate 39 (600 mg) as a white solid.

Compound of Example 21. Intermediate 39 (188 mg, 0.6 mmol), Intermediate 18 (85.6 mg, 0.3 mmol), K₂CO₃ (82.8 mg, 0.6 mmol) and PdCl₂(dppf)DCM (33.7 mg, 0.045 mmol) were placed in a 10 mL flask and 4 mL of dioxane. The mixture was degassed with Ar for 45 min, then stirred at 70° C. overnight. EtOAc (40 mL) was added, the mixture was filtered, extracted with EtOAc and washed with brine. The mixture was purified by silica gel column and compound of example 21 (42 mg) was obtained as a white solid. ¹HNMR: (400 MHz, DMSO-d₆, ppm); δ 9.15 (s, 1H); 8.38 (dd, J=1.3 Hz, 2H); 7.84 (s, 1H); 7.79 (d, J=2.0 Hz, 1H); 7.41 (d, J=2.0 Hz, 1H); 6.25 (s, 2H); 5.29 (t, J=1.5 Hz, 1H); 4.74 (m, J=0.8 Hz, 1H); 4.69 (m, J=1.9 Hz, 1H); 3.77 (m, 2H). MS (m/z): 390 [M+H].

### Example 22

### Compound of Structure

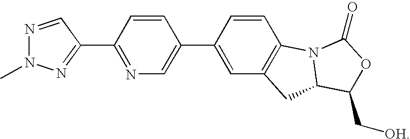

Scheme for Compound of Example 22:

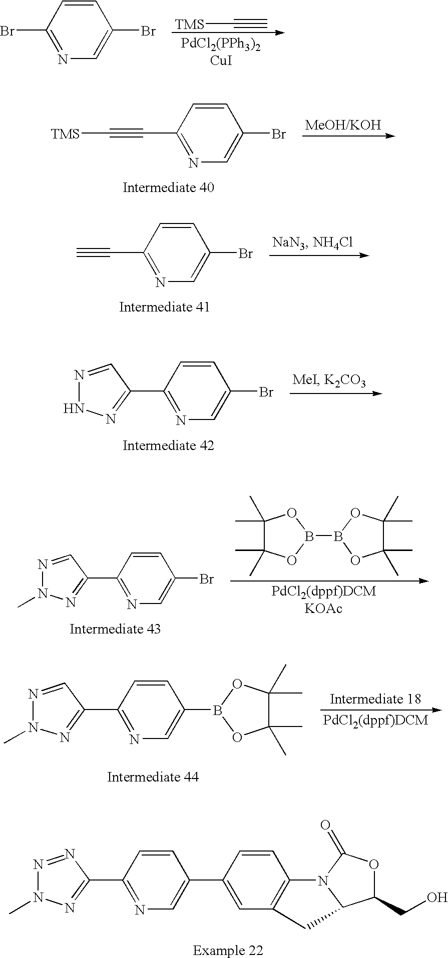

Intermediate 40. A mixture of 2,5-dibromopyridine (23.7 g, 100 mmol), PdCl₂(PPh₃)₂ (2.1 g, 3 mmol) and CuI (0.95 g, 5 mmol) in 200 mL of CH₃CN was degassed with Ar for 15 min and then ethynyltrimethylsilane (15.1 mL, 105 mmol) and i-Pr₂NH were added at 0° C. The mixture was stirred at r.t. o.n. Then the mixture was poured into 500 mL of water and extracted with EtOAc. The organic phase was washed with brine, dried and evaporated to give 23 g of crude product which was used directly for the next step.

Intermediate 41. A mixture of Intermediate 40 (23 g, crude), KOH (8.0 g) in 100 mL of MeOH and 40 mL of H₂O was stirred at r.t. for 1.5 h and evaporated. 200 mL of water was added and the mixture was extracted with EtOAc. The organic phase was washed with brine, dried and evaporated. Purification by silica gel column gave 9.8 g of Intermediate 41. MS (m/z): 183 [M+H].

Intermediate 42. Intermediate 41 (4.3 g, 23.6 mmol), NaN₃ (1.84 g, 28.3 mmol), and NH₄Cl (3.8 g, 70.8 mmol) were dissolved in DMF (50 mL) and the mixture was stirred at 120° C. o.n. The mixture was poured into ice-water (250 mL) and PH was adjusted to 2 with 6 N HCl (aq). The mixture was stirred for 1 h and filtered to give Intermediate 41 (3.7 g) as a white solid. MS (m/z): 226 [M+H].

Intermediate 43. Intermediate 42 (560 mg, 2.5 mmol), K₂CO₃ (340 mg, 2.5 mmol) and MeI (0.15 mL, 2.5 mmol) were dissolved in DMF (7 mL) and the mixture was stirred at 45° C. o.n. Water (50 mL) was added and the mixture was extracted with EtOAc, washed with brine, dried and evaporated. Purification by silica gel column gave Intermediate 43 (120 mg) as a white solid. MS (m/z): 240 [M+H].

Intermediate 44. Intermediate 43 (420 mg, 1.76 mmol), pinacol diborane (893 mg, 3.51 mmol), KOAc (517 mg, 5.28 mmol) and PdCl₂(dppf)DCM (127 mg, 0.17 mmol) were placed in a 10 mL flask and 5 mL of DMSO was added and the mixture was degassed with Ar for 45 min. The mixture was stirred at 80° C. o.n. Then 30 mL of H₂O and 40 mL of EtOAc were added to the mixture. The mixture was filtered, extracted with EtOAc and washed with brine. Purification by silica gel column gave Intermediate 44 (550 mg) as a white solid.

Compound of Example 22. Intermediate 44 (120 mg, 0.4 mmol), Intermediate 18 (58 mg, 0.2 mmol), K₂CO₃ (56 mg, 0.4 mmol) and PdCl₂(dppf)DCM (15 mg, 0.02 mmol) were mixed and placed in a 10 mL flask. Dioxane (3 mL) was added and the mixture was degassed with Ar for 45 min. The mixture was then stirred at 70° C. o.n. EtOAc (40 mL) was added to the mixture and the mixture was filtered. Purification by silica gel column gave compound of example 22 (38 mg) as a white solid. ¹H NMR (400 MHz, DMSO-d₆, ppm): δ 8.89 (d, J=2.0 Hz, 1H); 8.26 (s, 1H); 8.17 (dd, J=8.0, 2.4 Hz, 1H); 7.97 (d, J=8.0 Hz, 1H); 7.73 (s, 1H); 7.66 (d, J=8.0 Hz, 1H); 7.38 (d, J=8.4 Hz, 1H); 5.29 (t, J=2.0 Hz, 1H); 4.66˜4.73 (m, 2H); 4.25 (s, 3H); 3.78 (m, 2H); 3.26-3.31 (m, 2H). MS (m/z): 364 [M+H].

### Example 23

### Compound of Structure

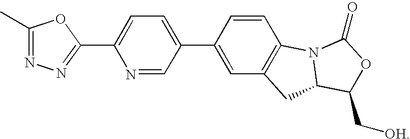

Scheme for Compound of Example 23:

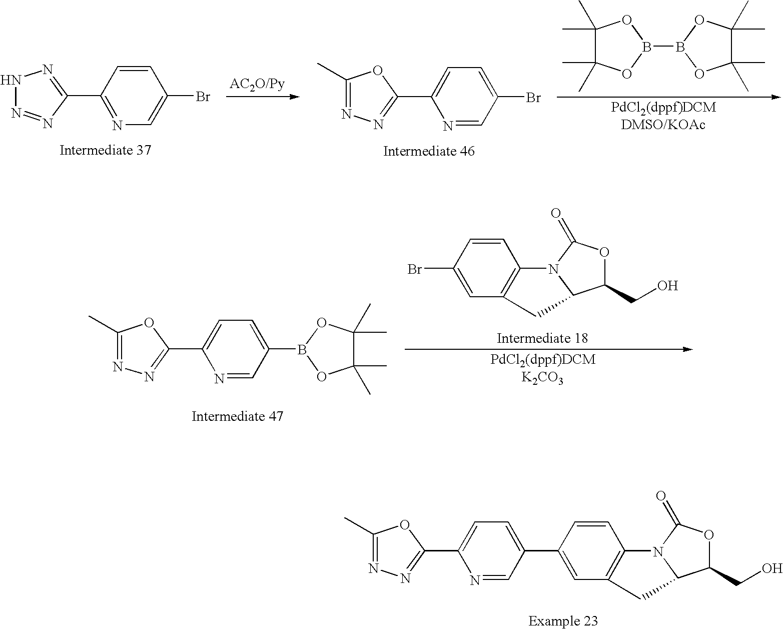

Intermediate 46. Intermediate 37 (5.3 g, 23.4 mol) was dissolved in Ac₂O (80 mL) at r.t. and pyridine (9.2 mL) was added. The mixture was heated to 130° C. for 1.5 h. Then the solution was poured into ice-water (400 mL) and PH was adjusted to 3-4 with 6 N HCl (aq). The mixture was stirred for 20 min and filtered. The desired product was collected as a white solid (4.25 g, 75%).

Intermediate 47. Intermediate 46 (1.0 g, 4.2 mmol), pinacol diborane (2.1 g, 8.3 mmol), KOAc (1.23 g, 12.5 mmol) and PdCl₂(dppf)DCM (312 mg, 0.4 mmol) were mixed and placed in a 25 mL flask. DMSO (10 mL) was added and the mixture was degassed with Ar for 45 min, then heated at 80° C. for 2.0 h. Water (30 mL) and EtOAc (10 mL) were added, and the mixture was filtered, extracted with EtOAc and washed with brine. Purification by silica gel column gave the desired product as a white solid (419 mg, 35%). ¹H NMR (400 MHz, DMSO-d₆, ppm): δ 8.91 (m, 1H); 8.22-8.25 (dd, J=7.6, 1.6 Hz, 1H); 8.14˜8.18 (dd, J=8.0, 1.2 Hz, 1H); 2.63 (s, 3H); 1.35 (s, 12H).

Compound of Example 23. Intermediate 47 (800 mg, 2.79 mmol), Intermediate 18 (500 mg, 1.76 mmol), K₂CO₃ (534 mg, 3.87 mmol) and PdCl₂(dppf)DCM (132 mg, 0.176 mmol) were mixed and placed in a 50 mL flask. Dioxane (30 mL) was added and the mixture was degassed with Ar for 45 min, then heated at 70° C. o.n. EtOAc was added and the mixture was filtered. Purification by silica gel column gave compound of example 23 (205 mg) as a white solid. ¹H (400 MHz, DMSO-d₆, ppm): δ 9.07 (d, J=6.0 Hz, 1H); 8.32 (dd, J=7.6, 6.4 Hz, 1H); 8.21 (d, J=8.4 Hz, 1H); 7.81 (s, 1H); 7.76 (d, J=8.0 Hz, 1H); 7.41 (d, J=8.4 Hz, 1H); 4.77 (m, 1H); 4.71 (dd, J=16.0, 7.6 Hz, 1H); 3.81 (dd, J=12.8, 3.6 Hz, 1H); 3.74 (dd, J=12.8, 3.6 Hz, 1H); 3.33 (dd, J=9.2, 3.2 Hz, 2H); 3.18 (s, 1H); 2.64 (s, 3H). MS (m/z): 365 [M+H].

### Example 24

### Compound of Structure

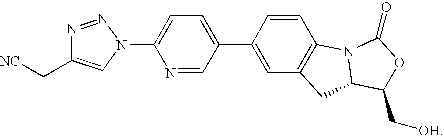

Scheme for Compound of Example 24:

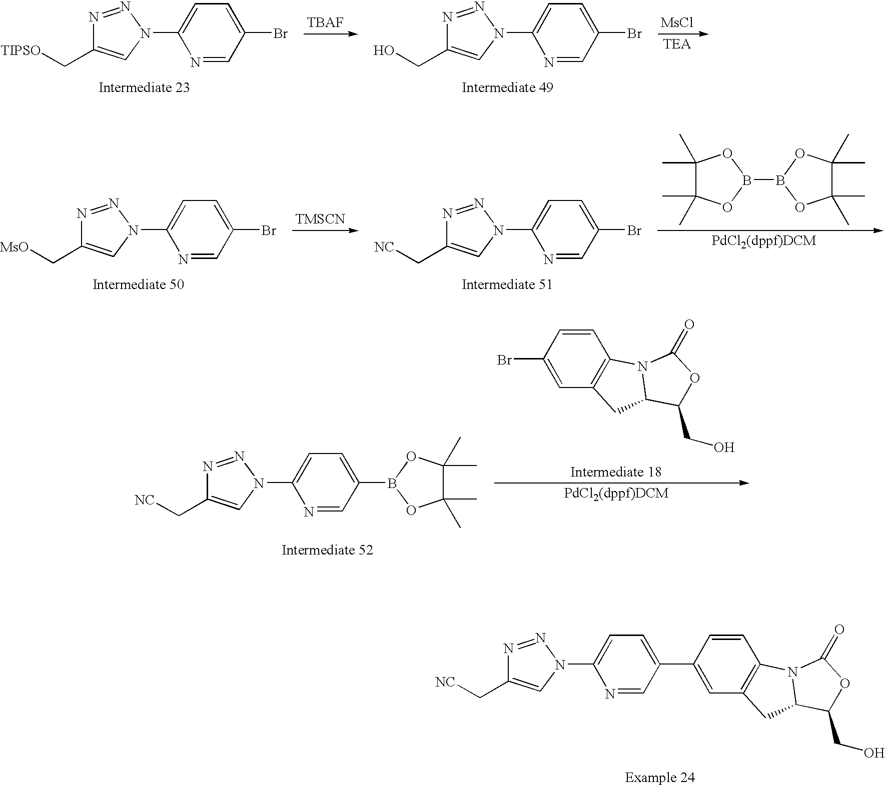

Intermediate 49. TBAF (767 mg, 2.4 mmol) was added to Intermediate 23 (500 mg, 1.2 mmol) in THF (10 mL) at 5° C. The mixture was allowed to warmed up to r.t. and stirred for 2 h. Volatiles were removed under vacuum. The residue was dissolved in EtOAc and washed with water, brine, and dried (MgSO₄). Solvent was removed under vacuum. Purification by chromatography (silica gel, petroleum ether:EtOAc=5:1) gave the product as a solid. MS (m/z): 255 [M+H].

Intermediate 50. MsCl (0.1 mL, 1.4 mmol) was added to Intermediate 49 (300 mg, 1.2 mmol) and Et₃N in DCM (10 mL) at 0° C. The reaction mixture was stirred at 0° C. for 30 min. Volatiles were removed under vacuum. The residue was dissolved in EtOAc and washed with water, brine, and dried (MgSO₄). Solvent was removed under vacuum and the product was obtained as a solid. MS (m/z): 333 [M+H].

Intermediate 51. TBAF in THF (1.9 mL, 1.9 mmol) was added to a mixture of Intermediate 50 (310 mg, 0.9 mmol) and TMSCN (184 mg, 1.8 mmol) in DMSO (5 mL). The reaction mixture was stirred at r.t. for 3 h. The mixture was taken into water, then filtered to afford the product as a solid. MS (m/z): 264 [M+H].

Intermediate 52. 4,4,4′,4′,5,5,5′,5′-Octamethyl-2,2′-bi(1,3,2-dioxaborolane) (750 mg, 2.96 mmol) and Intermediate 51 (520 mg, 1.97 mmol) were dissolved in 1,4-dioxane (10 mL). The solution was flushed with N², and KOAc (582 mg, 5.93 mmol) and PdCl₂(dppf) DCM (220 mg, 0.29 mmol) were added. The mixture was again flushed with N₂ 3 times, capped with a septum, and heated at 75° C. o.n. The reaction was then cooled down to r.t. The mixture was filtered and washed with DCM and EtOAc. The filtrate was concentrated in vacuo, and purified by silica gel column (2% MeOH/DCM). The product was obtained as an off white solid. MS (m/z): 312 [M+H].

Compound of Example 24. Intermediate 18 (90 mg, 0.31 mmol) and Intermediate 52 (118 mg, 0.38 mmol) were dissolved in 1,4-dioxane (3 mL). The solution was flushed with N2. K₂CO₃ (96 mg, 0.69 mmol) and PdCl₂(dppf) DCM (36 mg, 0.05 mmol) were added to the mixture. The mixture was again flushed with N₂ 3 times, capped with a septum, and heated at 75° C. o.n. The reaction was then cooled down to r.t., filtered, and washed with DCM and EtOAc. The filtrate was concentrated in vacuo, and purified by silica gel column (5% MeOH/DCM) to give the product as an off white solid. ¹H NMR (400 MHz, DMSO-d₆, ppm); δ 8.90 (dd, J=2.2, 0.6 Hz, 1H); 8.85 (s, 1H); 8.40 (dd, J=8.8, 2.4 Hz, 1H); 8.19 (d, J=9.2 Hz, 1H); 7.78 (s, 1H); 7.72 (dd, J=8.0, 2.0 Hz, 1H); 7.39 (d, J=8.4 Hz, 1H); 5.31 (t, J=5.6 Hz, 1H); 4.73 (m, 1H); 4.68 (m, 1H); 4.26 (s, 2H); 3.78 (m, 1H); 3.70 (m, 1H); 3.29 (d, J=8.8 Hz, 2H). MS (m/z): 389 [M+H].

### Example 25

### Compound of Structure

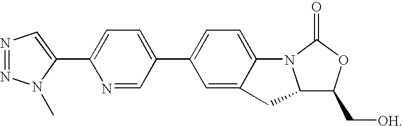

Scheme for Compound of Example 25:

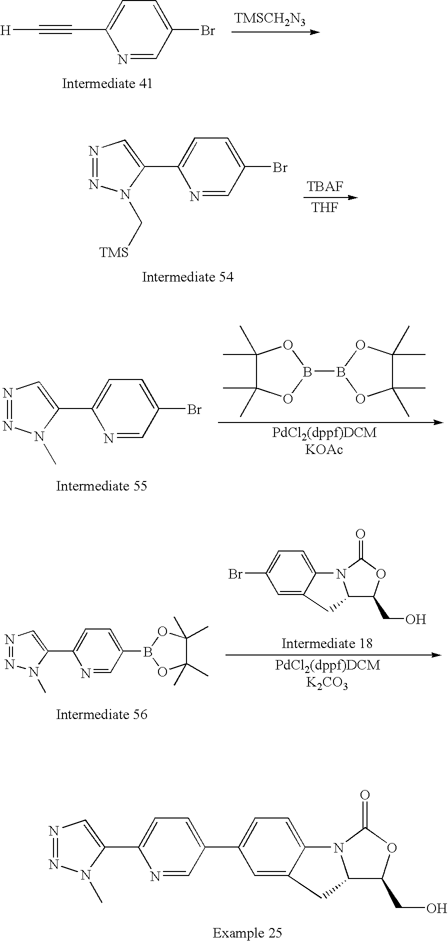

Intermediate 54. (Azidomethyl)trimethylsilane (0.6 mL, 4.0 mmol) was added to Intermediate 41 (500 mg, 2.76 mmol) in toluene (10 mL), and the reaction mixture was warmed up to 80° C. and stirred o.n. Solvent was removed under vacuum and the residue was purified by column chromatography (petroleum ether:EtOAc=6:1). The product was obtained as a yellow solid. MS (m/z): 311, 313 [M+H].

Intermediate 55. TBAF (775 mg, 2.46 mmol) was added with stirring to Intermediate 54 (383 mg, 1.23 mmol) in dry THF (10 mL) at r.t. After 2 h the solvent was removed under vacuum and water (10 mL) was added. The mixture was extracted with EtOAc. The organic layer was washed with brine, and dried (Na₂SO₄). Solvent was evaporated under vacuum to afford the product as a yellow solid. ¹H NMR (400 MHz, CDCl₃, ppm): δ 8.63 (d, J=2.4 Hz, 1H), 8.10 (s, 1H), 8.08 (d, J=8.8 Hz, 1H), 7.90 (dd, J=8.8, 2.4 Hz, 1H), 4.17 (s, 3H). MS (m/z): 239, 241 [M+H].

Intermediate 56. 4,4,4′,4′,5,5,5′,5′-Octamethyl-2,2′-bi(1,3,2-dioxaborolane) (486 mg, 1.88 mmol), Intermediate 55 (300 mg, 1.25 mmol), KOAc (368 mg, 3.75 mmol), and PdCl₂(dppf) DCM (143 mg, 0.19 mmol) were dissolved in DMF (5 mL). The mixture was flushed with N₂ 3 times, capped with a septum, and heated at 70° C. o.n. The reaction was then cooled down to r.t., filtered, and washed with EtOAc. The filtrate was washed with water, brine, and dried (Na₂SO₄). Volatiles were removed under vacuum and the residue was purified by column chromatography (petroleum ether:EtOAc=2:1). The product was obtained as a yellow solid. MS (m/z): 287 [M+H].

Compound of Example 25. Intermediate 18 (70 mg, 0.25 mmol), intermediate 56 (79 mg, 0.28 mmol), K₂CO₃ (76 mg, 0.55 mmol), and PdCl₂(dppf) DCM (19 mg, 0.03 mmol) were dissolved in 1,4-dioxane (3 mL). The mixture was flushed with N₂ 3 times, capped with a septum, and heated at 70° C. for 5 h. The reaction was then cooled down to r.t., filtered, and washed with DCM and EtOAc. The filtrate was concentrated in vacuo, and purified by silica gel column (3% MeOH/DCM) to give the product as a pale yellow solid. NMR (400 MHz, DMSO-d₆, ppm): δ8.89 (d, J=2.0 Hz, 1H); 8.60 (s, 1H); 8.17 (dd, J=2.4 Hz, 1H); 8.09 (d, J=8.0 Hz, 1H); 7.72 (s, 1H); 7.68 (d, J=8.0 Hz, 1H); 7.38 (d, J=8.4 Hz, 1H); 5.29 (t, J=2.0 Hz, 1H); 4.66-4.73 (m, 2H); 4.13 (s, 3H); 3.26-3.31 (m, 2H). MS (m/z): 364 [M+H].

### Example 26

### Compound of Structure

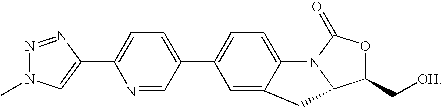

Scheme for Compound of Example 26:

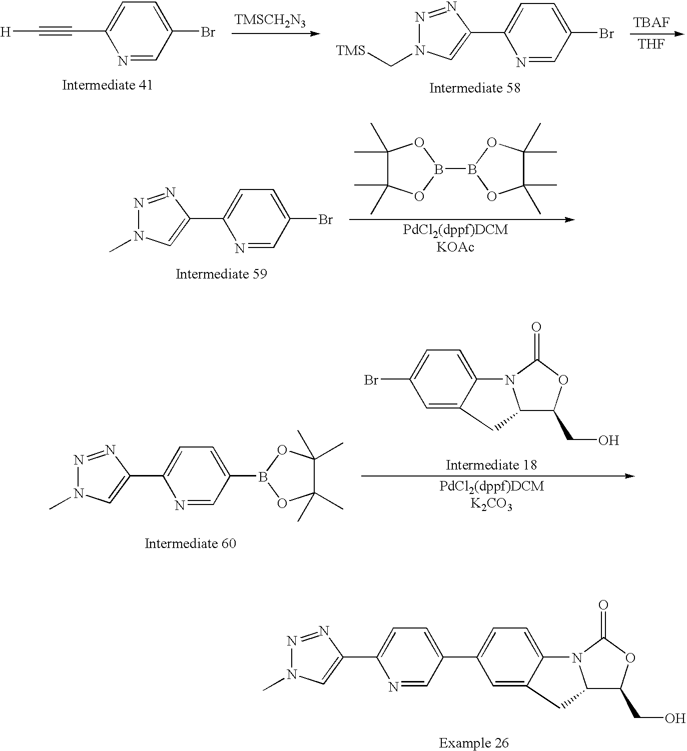

Intermediate 58. (Azidomethyl)trimethylsilane (0.6 mL, 4.0 mmol) was added to Intermediate 41 (500 mg, 2.76 mmol) in toluene (10 mL), and the reaction mixture was warmed up to 80° C. and stirred o.n. Solvent was removed under vacuum and the residue was purified by column chromatography (petroleum ether:EtOAc=6:1). The product was obtained as a yellow solid. MS (m/z): 311, 313 [M+H].

Intermediate 59. TBAF (860 mg, 2.74 mmol) was added with stirring to Intermediate 58 (425 mg, 1.37 mmol) in dry THF (10 mL) at r.t. After 2 h solvent was removed under vacuum and water (10 mL) was added. The mixture was extracted with EtOAc. The organic layer was washed with brine, and dried (Na₂SO₄). Solvent was evaporated under vacuum to afford the product as a yellow solid. ¹H NMR (400 MHz, CDCl₃, ppm): δ 8.76 (d, J=2.4 Hz, 1H), 8.00 (s, 1H), 7.93 (dd, J=8.0, 2.4 Hz, 1H), 7.53 (d, J=8.0 Hz, 1H), 4.40 (s, 3H). MS (m/z): 239, 241 [M+H].

Intermediate 60. 4,4,4′,4′,5,5,5′,5′-Octamethyl-2,2′-bi(1,3,2-dioxaborolane) (648 mg, 2.55 mmol), Intermediate 59 (400 mg, 1.67 mmol), KOAc (490 mg, 5.0 mmol), and PdCl₂(dppf) DCM (190 mg, 0.25 mmol) were dissolved in DMF (5 mL). The mixture was flushed with N₂ 3 times, capped with a septum, and heated at 60° C. o.n. The reaction was then cooled down to r.t., filtered, and washed with EtOAc. The filtrate was washed with water, brine, and dried (Na₂SO₄). Volatiles were removed under vacuum, and the residue was purified by column chromatography (petroleum ether:EtOAc=2:1) to afford the product as yellow solid. MS (m/z): 287 [M+H].

Compound of Example 26. Intermediate 18 (90 mg, 0.32 mmol), Intermediate 60 (109 mg, 0.38 mmol), K₂CO₃ (98 mg, 0.71 mmol), and PdCl₂(dppf) DCM (25 mg, 0.03 mmol) were dissolved in 1,4-dioxane (3 mL). The mixture was flushed with N₂ 3 times, capped with a septum, and heated at 70° C. for 8 h. The reaction was then cooled down to r.t., filtered, and washed with DCM and EtOAc. The filtrate was concentrated in vacuo, and purified by silica gel (5% MeOH/DCM) to afford the product as a yellow solid. ¹H NMR (400 MHz, DMSO, ppm): δ 9.03 (d, J=2.4 Hz, 1H); 8.34 (s, 1H); 8.24 (dd, J=8.4, 2.8 Hz, 1H); 8.00 (d, J=8.4 Hz, 1H); 7.77 (s, 1H); 7.73 (d, J=8.0 Hz, 1H); 7.39 (d, J=8.4 Hz, 1H); 5.30 (t, J=5.6 Hz, 1H); 4.73 (m, 1H); 4.68 (m, 1H); 4.37 (s, 3H); 3.77 (m, 1H); 3.72 (m, 1H); 3.31 (br, 1H); 3.29 (br, 1H). MS (m/z): 364 [M+H].

### Example 27

### Compound of Structure

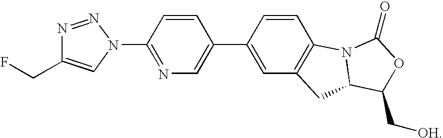

Scheme for Compound of Example 27:

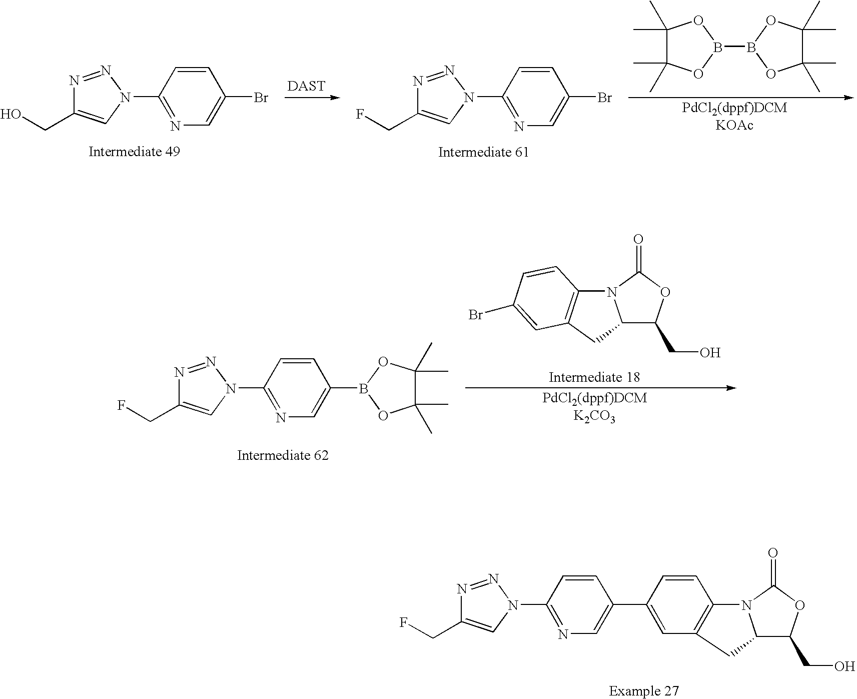

Intermediate 61. Diethylamino sulphur trifluoride (1.61 g, 3.92 mmol) was added slowly to intermediate 49 (0.5 g, 1.96 mmol) in THF (8 mL) at 0° C. The solution was stirred for about 1 h. Then the mixture was concentrated, extracted with ethyl acetate, washed with water and brine, dried (MgSO₄). Solvent was removed and the product was obtained as a white solid (0.42 g, 84%). MS (m/z): 257 [M+H]

Intermediate 62. 4,4,4′,4′,5,5,5′,5′-octamethyl-2,2′-bi(1,3,2-dioxaborolane) (490 mg, 1.9 mmol), KOAc (300 mg, 3.0 mmol) and PdCl₂(dppf)DCM (80 mg, 0.1% equiv.) were added to a stirred solution of intermediate 63 (250 mg, 0.97 mmol) in DMF (15 mL). The mixture was degassed with Ar before heated to 80° C. After 4-5 h, the mixture was diluted with water and then filtered. The filtrate was extracted with DCM, and the organic layer was combined, washed with water, brine and dried (MgSO₄). After removing the solvent, the residue was purified by column chromatography (EtOAc/Petroleum Ether=1:1). The product was obtained as a yellow solid (170 mg, 64%). MS (m/z): 305 [M+H].

Compound of Example 27. Intermediate 18 (90 mg, 0.31 mmol) and Intermediate 62 (116 mg, 0.38 mmol) were dissolved in 1,4-dioxane (3 mL). The solution was flushed with N2. K₂CO₃ (96 mg, 0.69 mmol) and PdCl₂(dppf) DCM (36 mg, 0.05 mmol) were added. The mixture was degassed with N², and then heated to 75° C. After stirring for about 4 h, the reaction was cooled down to r.t. and filtered. The filtrate was washed with water, brine and extracted with EtOAc. The organic layers were collected and concentrated in vacuo. The residue was purified by column chromatography (5% MeOH/DCM). The product was obtained as an off white solid. ¹H NMR (400 MHz, DMSO-d6, ppm): δ 9.11 (d, J=3.2 Hz, 1H); 8.91 (d, J=2.0 Hz, 1H); 8.41 (dd, J=8.6, 2.4 Hz, 1H); 8.39 (d, J=8.8 Hz, 1H); 7.78 (s, 1H); 7.73 (d, J=8.0 Hz, 1H); 7.39 (d, J=8.0 Hz, 1H); 5.56 (s, 1H); 5.53 (s, 1H); 5.31 (t, J=5.6 Hz, 1H); 4.68-4.75 (m, 2H); 3.71-3.78 (m, 2H); 3.29 (s, 1H). MS (m/z): 382 [M+H].

### Example 28

### Compound of Structure

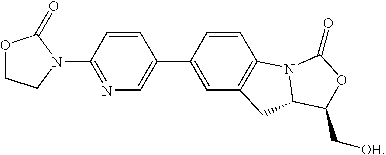

Scheme for Compound of Example 28:

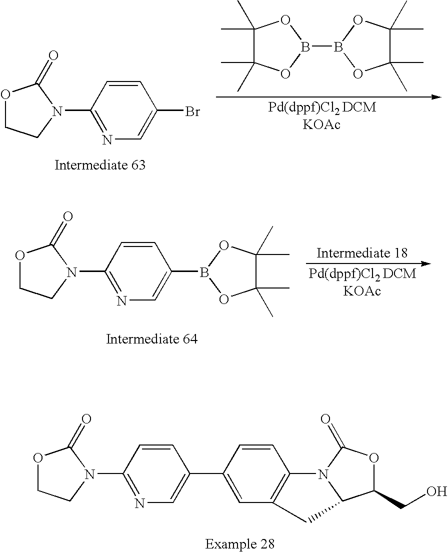

Intermediate 64. Intermediate 63 (214 mg, 0.88 mmol) was dissolved in 5 mL DMSO. KOAc (260 mg, 2.65 mmol) was added, followed by Pd(dppf)Cl₂ (66 mg, 0.09 mmol [symbol] and bis(pinacolato)diboron (450 mg 1.77 mmol). The mixture was stirred for 10 min, flushed with Ar 3 times, and then heated at 80° C. for 2 h. The reaction was then cooled down to r.t., diluted with ice/water (50 mL) and filtered. The organic layers were combined, washed with water (100 mL), brine (100 mL) and dried (Na₂SO₄). Solvent was removed under vacuum and the residue was purified by column chromatography (20% EtOAc/Petroleum Ether) to afford 120 mg of the desired product.

Compound of Example 28. Intermediate 63 (80 mg, 0.28 mmol) was dissolved in 2.5 mL of 1,4-dioxane and 0.5 mL of water. Intermediate 18 (65 mg, 0.23 mmol) was added, followed by Pd(dppf)Cl₂ (21 mg, 0.03 mmol) and K₂CO₃ (97 mg, 0.70 mmol). The flask was purged with Ar and degassed for 30 min. The mixture was heated to 70° C. After 3 h, the mixture was cooled to r.t., poured into ice/water (25 mL) and filtered. The filtrate was extracted with ethyl acetate (20 mL×3). The organic layers were collected, washed with water (20 mL), brine (20 mL) and dried (Na₂SO₄). Volatiles were removed under vacuum, and the crude product was purified by column chromatography (5% MeOH/DCM) to afford 32 mg of the title compound. ¹HNMR: (400 MHz, DMSO-d₆, ppm): δ 8.66 (d, J=1.2 Hz, 1H); 8.10-8.16 (m, 2H); 7.66 (s, 1H); 7.56 (d, J=8.0 Hz, 2H); 7.33 (d, J=8.0 Hz, 1H); 5.28 (t, J=5.6 Hz, 1H); 4.70˜4.74 (m, 1H); 4.61-4.66 (m, 1H); 4.48 (t, J=8.0 Hz, 2H); 4.21 (t, J=8.0 Hz, 2H); 4.76-4.80 (m, 1H); 4.76˜4.80 (m, 1H); 3.67˜3.72 (m, 1H); 3.29 (d, J=8.0 Hz, 1H). MS (m/z): 368 [M+H].

### Example 29

### Compound of Structure

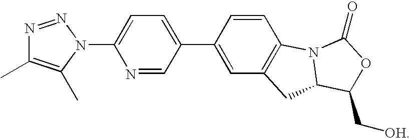

Scheme for Compound of Example 29:

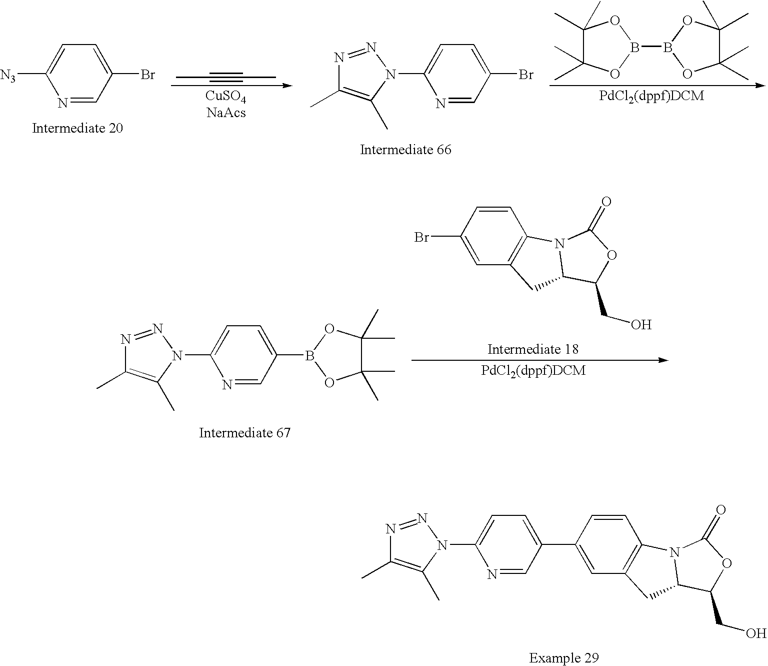

Intermediate 66. Intermediate 20 (400 mg, 2.0 mmol), but-2-yne (2.0 mL) and toluene (2.0 mL) were placed in a pressured tube and heated at 120° C. o.n. Volatiles were removed under vacuum. The residue was purified by column chromatography (petroleum ether: EtOAc=20:1). The product was obtained as a solid. MS (m/z): 253 [M+H].

Intermediate 67. 4,4,4′,4′,5,5,5′,5′-octamethyl-2,2′-bi(1,3,2-dioxaborolane) (376 mg, 1.5 mmol) and Intermediate 66 (250 mg, 1.0 mmol) were dissolved in 1,4-dioxane (10 mL). The solution was flushed with N2. KOAc (290 mg, 3.0 mmol) was added, followed by PdCl₂(dppf) DCM (111 mg, 0.15 mmol). The mixture was again flushed with N₂ 3 times, capped with a septum, and heated at 70° C. o.n. The reaction was then cooled down to r.t., filtered, and washed with DCM and EtOAc. The filtrate was concentrated in vacuo, and purified by column chromatography (2% MeOH/DCM). The product was obtained as an off white solid. MS (m/z): 301 [M+H].

Compound of Example 29. Intermediate 18 (100 mg, 0.35 mmol) and Intermediate 67 (126 mg, 0.42 mmol) were dissolved in 1,4-dioxane (2 mL). The solution was flushed with N2. K₂CO₃ (97 mg, 0.70 mmol) was added, followed by PdCl₂(dppf) DCM (52 mg, 0.07 mmol). The mixture was again flushed with N₂ 3 times, capped with a septum, and heated at 80° C. o.n. The reaction was then cooled down to r.t., filtered, and washed with DCM and EtOAc. The filtrate was concentrated in vacuo, and purified by column chromatography (5% MeOH/DCM). The product was obtained as an off white solid. ¹H NMR (400 MHz, CDCl₃, ppm): δ 8.70 (s, 1H); 8.04 (t, J=9.8 Hz, 2H); 7.56 (m, 2H); 7.48 (s, 1H); 4.84 (m, 1H); 4.66 (s, 1H); 4.11 (d, J=12.8 Hz, 1H); 3.92 (d, J=10.4 Hz, 1H); 3.39 (m, 1H); 3.23 (m, 1H); 2.60 (s, 3H); 2.37 (s, 3H). MS (m/z): 378 [M+H].

### Example 30

### Compound of Structure

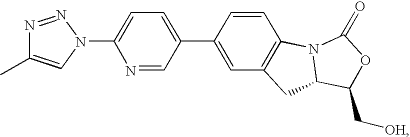

Scheme for Compound of Example 30:

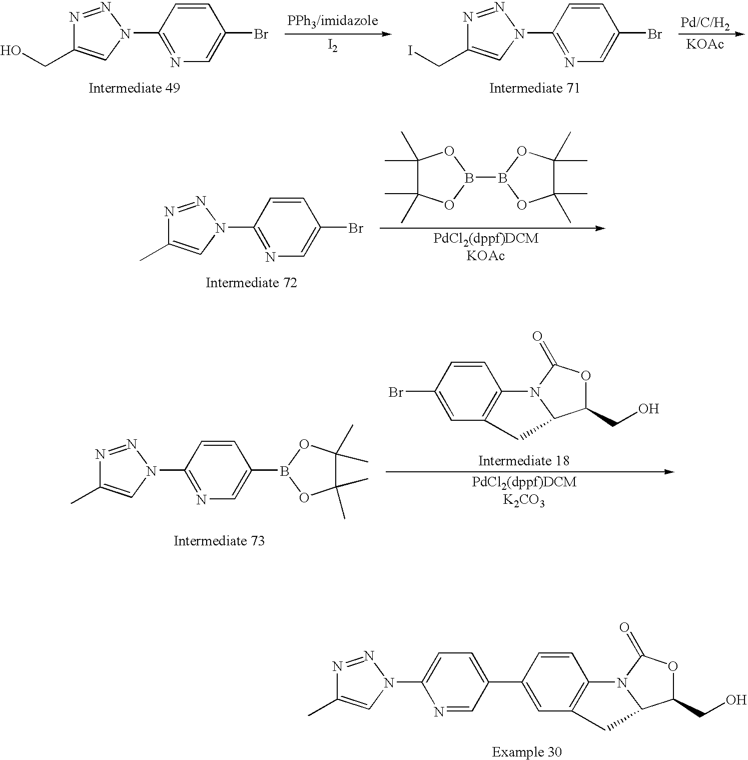

Intermediate 71. To a solution of PPh₃ (703 mg, 2.68 mmol) and imidazole (405 mg, 5.96 mmol) in DCM (28 mL) was slowly added a solution of iodine (754 mg, 2.98 mmol) in 42 mL of DCM at 0° C. After 15 min, a solution of Intermediate 49 (190 mg, 0.74 mmol) in 3.0 mL of DCM was added into the mixture. The reaction mixture was stirred for 20 min under ice-water followed by 16 h at ambient temperature. The reaction mixture was quenched with water and DCM was removed. The mixture was extracted with EtOAc, The EtOAc extracts were washed with water, brine, dried (MgSO₄) and concentrated. The residue was purified by column chromatography to give Intermediate 71 as a white solid (246 mg, 91%). MS (m/z): 365 [M+H]

Intermediate 72. Intermediate 71 (500 mg, 1.37 mmol) was dissolved in MeOH (60 mL). KOAc (202 mg, 2.1 mmol) was added, followed by Pd/C (Cat.). The reaction mixture was stirred under H₂ at r.t. o.n. Solvent was removed and EtOAc was added, and the mixture was filtered. The filtrate was washed with brine, dried (Na₂SO₄) and concentrated. The residue was purified by crystallization to give the product as a white solid (198 mg, 60%). MS (m/z): 239 [M+H].

Intermediate 73. Intermediate 72 (228 mg, 0.95 mmol), pinacol diborane (485 mg, 1.9 mmol), KOAc (280 mg, 2.85 mmol) and PdCl₂(dppf)DCM (72 mg, 0.095 mmol) were mixed and placed in a 10 mL flask. DMSO (3.5 mL) was added and the mixture was degassed with Ar for 45 min. The mixture was heated at 80° C. for 3.5 h. H₂O (30 mL) and EtOAc (10 mL) was added, and the mixture was filtered. The filtrate was extracted with EtOAc and washed with brine, dried (Na₂SO₄) and concentrated. Purification by column chromatography gave the desired product (96 mg, 35%).

Compound of Example 30. Intermediate 73 (86 mg, 0.30 mmol), Intermediate 18 (71 mg, 0.25 mmol), K₂CO₃ (69 mg, 0.5 mmol) and PdCl₂(dppf)DCM (28 mg, 0.037 mmol) were mixed and placed in a 10 mL flask. Dioxane (3 mL) was added and the mixture was degassed with Ar for 45 min. The mixture was heated at 70° C. o.n. EtOAc (40 mL) was added, and the mixture was filtered. The filtrate was extracted with EtOAc and washed with brine, dried (Na₂SO₄) and concentrated. Purification by column chromatography gave the desired product (21 mg, 23%). ¹HNMR (400 MHz, DMSO-d₆, ppm): δ 8.84 (s, 1H); 8.60 (s, 1H); 8.32˜8.35 (d, J=10.8 Hz, 1H); 8.12-8.14 (d, J=8.4 Hz, 1H); 7.74 (s, 1H); 7.67˜7.70 (d, J=8.0 Hz, 1H); 7.36-7.38 (d, J=8.4 Hz, 1H); 5.28˜5.31 (t, J=11.6 Hz, 1H); 4.65-4.74 (m, 2H); 3.7-3.77 (m, 2H); 3.28-3.30 (d, J=9.2 Hz, 2H); 2.35 (s, 3H). MS (m/z): 364 [M+H].

### Example 31

### Compound of Structure

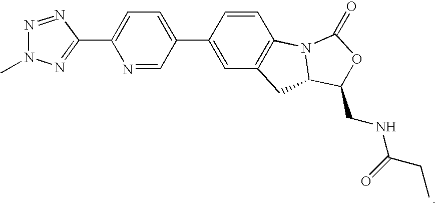

Scheme for Compound of Example 31:

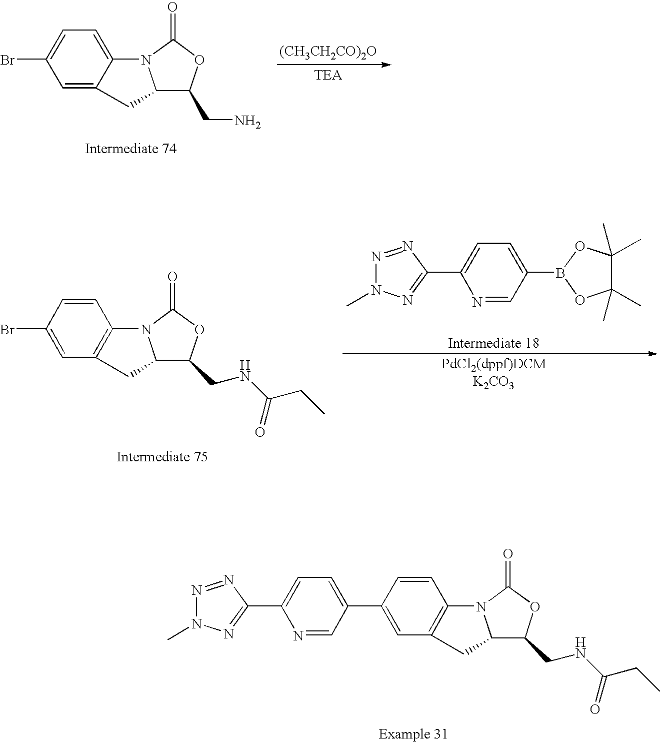

Intermediate 75. Acetic anhydride (335 μL, 2.6 mmol) was added to a mixture of Intermediate 74 (368 mg, 1.3 mmol) and Triethyl amine (360 μL, 2.6 mmol) in THF (5 mL) at r.t. After stirring for 1 h, water was added and the mixture was extracted with EtOAc. The organic layer was washed with sat. NH₄Cl, brine, and dried (Na₂SO₄). Solvent was removed under vacuum to afford the product as a pale yellow solid and used directly for the next step.

Compound of Example 31. 2-(2-Methyl-2H-tetrazol-5-yl)-5-(4,4,5,5-tetramethyl-1,3,2-dioxaborolan-2-yl)pyridine (344 mg, 1.20 mmol), Intermediate 75 (340 mg, 1.0 mmol), K₂CO₃ (276 mg, 2.0 mmol) and PdCl₂(dppf) DCM (75 mg, 0.10 mmol) were dissolved in 1,4-dioxane (5 mL). The mixture was flushed with N₂ 3 times, capped with a septum, and heated at 80° C. o.n. The reaction was then cooled down to r.t., filtered, and washed with DCM and EtOAc. The filtrate was concentrated in vacuo, and purified by column chromatography (1% MeOH/DCM). The product was obtained as a white solid. ¹H NMR (400 MHz, DMSO-d₆, ppm): δ 9.12 (dd, J=2.0, 0.4 Hz, 1H); 8.31 (m, 3H); 7.81 (s, 1H); 7.76 (dd, J=8.0, 1.6 Hz, 1H); 7.34 (d, J=8.4 Hz, 1H); 4.77 (m, 1H); 4.57 (dd, J=16.8, 9.2 Hz, 1H); 4.46 (s, 3H); 3.56 (t, J=5.2 Hz, 2H); 3.28 (d, J=8.8 Hz, 2H); 2.17 (dd, J=15.2, 7.6 Hz, 2H); 1.03. (t, J=7.6 Hz, 3H). MS (m/z): 420 [M+H].

### Example 32

### Compound of Structure

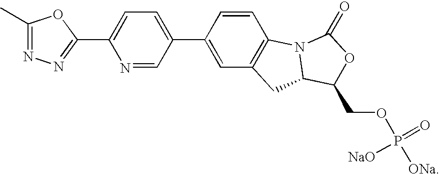

Scheme for Compound of Example 32:

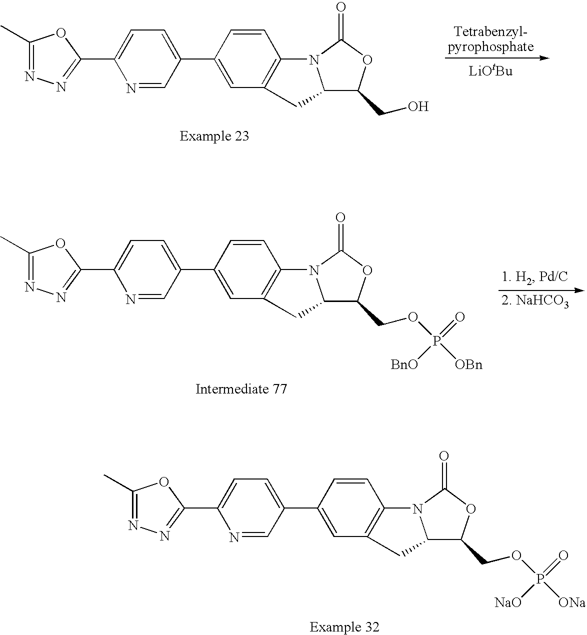

Intermediate 77. This compound was prepared analogously to the procedure in preparation of Compound of Example 4 (Method A), except that Compound of Example 1 was substituted for Compound of Example 23. Reagents used: LiO^{t}Bu in THF (1.5 mL, 1.5 mmol), Intermediate 1 (219 mg, 0.6 mmol) in DMF (4 mL), tetrabenzyl diphosphate (810 mg, 1.5 mmol) in DMF (4 mL). MS (m/z): 625 [M+H].

Compound of Example 32. This compound was prepared analogously to the procedure in preparation of Compound of Example 5 (Method B), except that Intermediate 14 was substituted for Intermediate 77. Reagents used: NaHCO₃ (12.7 mg, 0.15 mmol) in water (1 mL), Pd/C (15 mg), Intermediate 77 (50 mg, 0.08 mmol) in THF (1 mL). ¹H NMR (400 MHz, D₂O, ppm): δ 8.49 (s, 1H); 7.91 (d, J=8.0 Hz, 1H); 7.81 (d, J=8.4 Hz, 1H); 7.36 (s, 1H); 7.30 (d, J=8.8 Hz, 1H); 7.13 (d, J=8.8 Hz, 1H); 4.82 (m, 1H); 4.66 (m, 1H); 4.08 (m, 2H); 3.28 (m, 1H); 3.09 (m, 1H); 2.55 (s, 3H). MS (m/z): 445 [M-2Na+3H] (in agreement with the ionized diphosphoric acid form).

### Example 33

### Compound of Structure

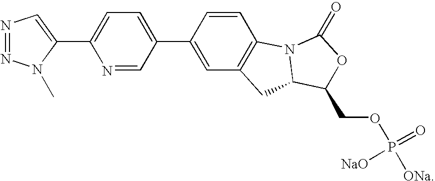

Scheme for Compound of Example 33:

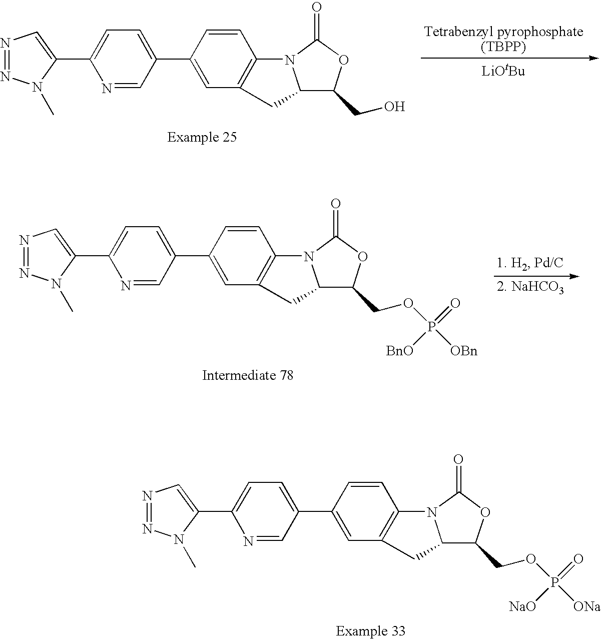

Intermediate 78. This intermediate was prepared analogously to the procedure in preparation of Compound of Example 4 (Method A), except that Compound of Example 1 was substituted for Compound of Example 25. Reagents used: LiO^{t}Bu in THF (1.93 mL, 1.93 mmol), Compound of example 25 (280 mg, 0.77 mmol) in DMF (3 mL), tetrabenzyl diphosphate (1.04 g, 1.93 mmol) in DMF (2 mL).

Compound of Example 33. This compound was prepared analogously to the procedure in preparation of Compound of Example 5 (Method B), except that Intermediate 14 was substituted for Intermediate 78. Reagents used: NaHCO₃ (12.8 mg, 0.15 mmol) in water (1 mL), Pd/C (20 mg), Intermediate 78 (50 mg, 0.08 mmol) in THF (1 mL). ¹H NMR (400 MHz, D₂O, ppm): δ 8.39 (s, 1H); 8.09 (s, 1H); 7.81 (d, J=8.0 Hz, 1H); 7.67 (d, J=8.4 Hz, 1H); 7.30 (s, 1H); 7.29 (s, 1H); 7.15 (d, J=8.8 Hz, 1H); 4.62 (m, 2H); 4.02 (m, 5H); 3.29 (dd, J=16.0, 8.4 Hz, 1H); 3.08 (dd, J=12.0, 4.0 Hz, 1H). MS (m/z): 444 [M-2Na+3H] (in agreement with the ionized diphosphoric acid form).

### Example 34

### Compound of Structure

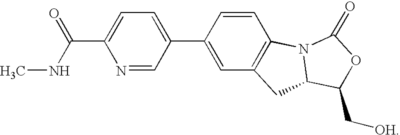

Scheme for Compound of Example 34:

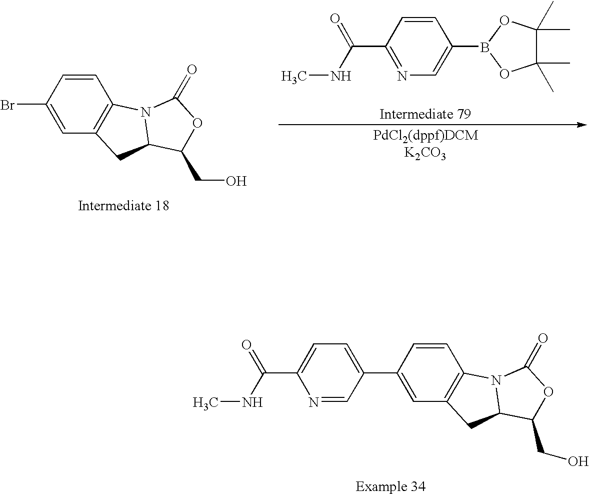

Compound of Example 34. The Compound was prepared as described for the last step in the preparation of Compound of Example 30, except that Intermediate 73 was substituted for Intermediate 79. ¹H NMR (300 MHz, CD₃OD, ppm): 8.73 (br, 1H), 8.11 (m, 1H); 8.02 (m, 1H), 7.53-7.46 (m, 3H), 4.80-4.71 (m, 1H), 4.65-3.60 (m, 1H), 3.96-3.91 (dd, J=4.20 and 12.30 Hz, 1H), 3.87-3.81 (dd, J=3.90 and 12.60, 1H), 3.44-3.32 (m, 1H), 3.26-3.16 (m, 1H). MS (m/z): 340 [M+H].

### Example 35

### Compound of Structure

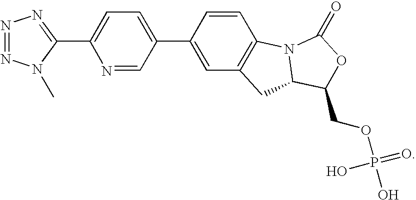

Scheme for Compound of Example 35:

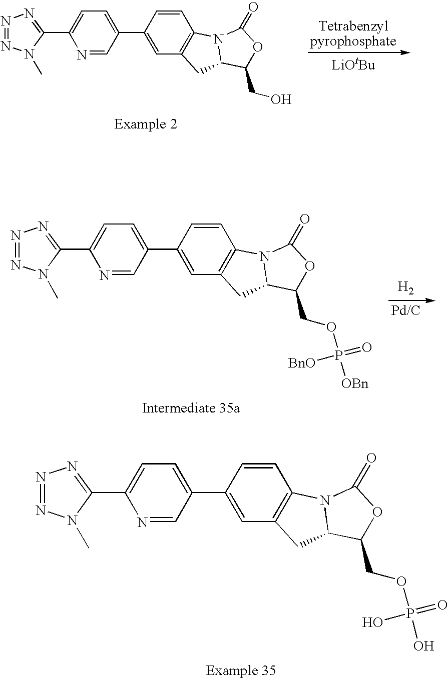

Compound of Example 35. This compound was prepared analogously to the procedure in preparation of Compound of Example 4 (Method A), except that Compound of Example 1 was substituted for Compound of Example 2. ¹H NMR (300 MHz, DMSO-d₆, ppm): 9.03 (s, 1H), 8.28-8.16 (m, 2H), 7.87 (s, 1H), 7.67 (d, J=7.80 Hz, 1H), 7.39-7.36 (m, 1H), 5.00-4.70 (m, 1H), 4.41 (s, 3H), 4.20-3.90 (m, 2H), 3.70-3.00, overlapped with H₂O, 2H). MS (m/z): 445 [M+H].

### Example 36

### Compound of Structure

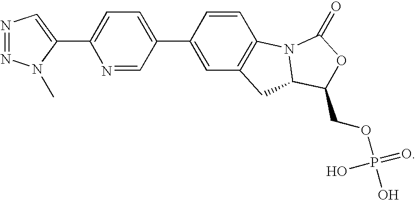

Scheme for Compound of Example 36:

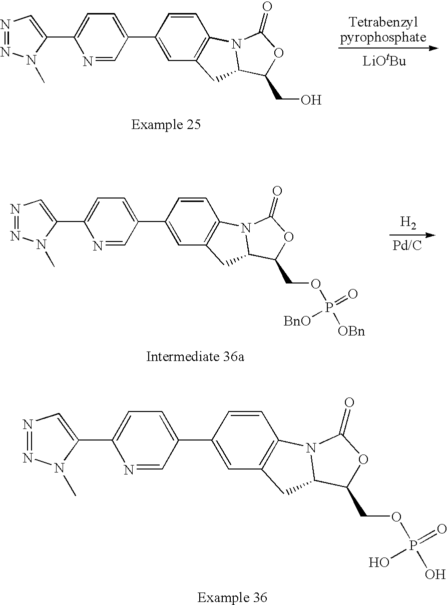

Compound of Example 36. This compound was prepared analogously to the procedure in preparation of Compound of Example 4 (Method A), except that Compound of Example 1 was substituted for Compound of Example 25. MS (m/z): 444 [M+H].

### Example 37

### Compound of Structure

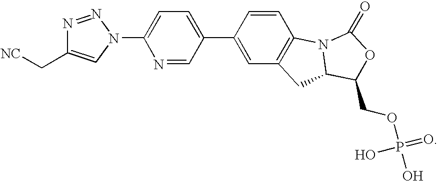

Scheme for Compound of Example 37:

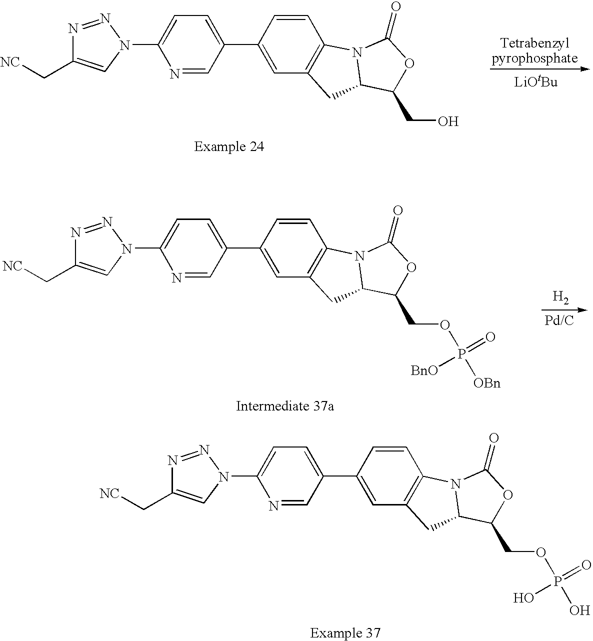

Compound of Example 37. This compound was prepared analogously to the procedure in preparation of Compound of Example 4 (Method A), except that Compound of Example 1 was substituted for Compound of Example 24. MS (m/z): 469 [M+H].

### Example 38

### Compound of Structure

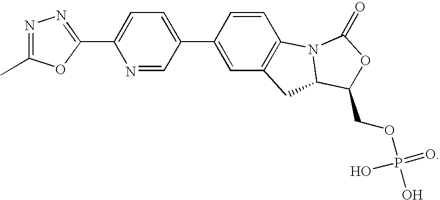

Scheme for Compound of Example 38:

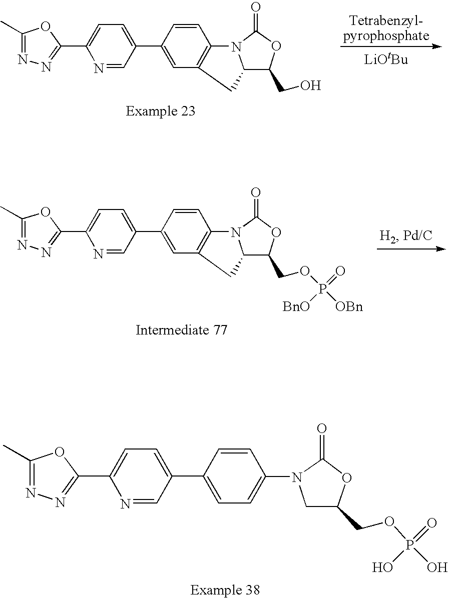

Compound of Example 38. This compound was prepared analogously to the procedure in preparation of Compound of Example 4 (Method A), except that Compound of Example 1 was substituted for Compound of Example 23. MS (m/z): 445 [M+H].

### Example 39

### Compound of Structure

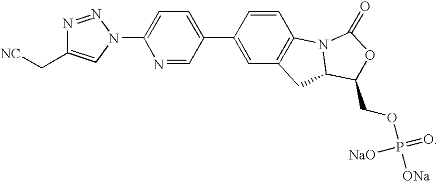

Scheme for Compound of Example 39:

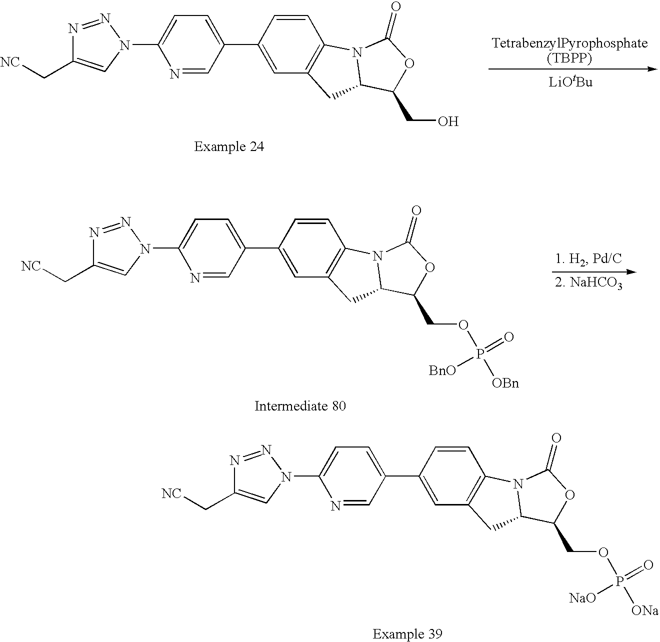

Intermediate 80. This compound was prepared according to a similar procedure in preparation of Compound of Example 4 (Method A), except that Compound of Example 1 was substituted for Compound of Example 24. Reagents used: 1 M LiO^{t}Bu in THF (2.5 mL, 2.5 mmol), Compound of Example 24 (400 mg, 1.03 mmol) in DMF (5 mL), tetrabenzyl diphosphate (1.4 g, 2.5 mmol) in DMF (5 mL). MS (m/z): 649 [M+H].

Compound of Example 39. This compound was prepared according to a similar procedure in preparation of Compound of Example 5 (Method B), except that Intermediate 14 was substituted for Intermediate 80. NaHCO₃ (24.4 mg, 0.29 mmol) in water (2 mL), Pd/C (30 mg), Intermediate 80 (100 mg, 0.15 mmol) in THF (2 mL). ¹H NMR (400 MHz, D₂O+NaHCO₃, ppm); [symbol] 8.32 (s, 1H); 8.26 (s, 1H); 7.86 (d, J=8.4 Hz, 1H); 7.62 (d, J=8.8 Hz, 1H); 7.26 (s, 1H); 7.19 (d, J=8.0 Hz, 1H); 7.05 (d, J=8.0 Hz, 1H); 4.03 (m, 4H); 3.24 (m, 1H); 3.04 (m, 1H); 4.80 (overlapped by D₂O, 2H). MS (m/z): 469 [M-2Na+3H] (in agreement with the ionized diphosphoric acid form).

### Example 40

### Compound of Structure

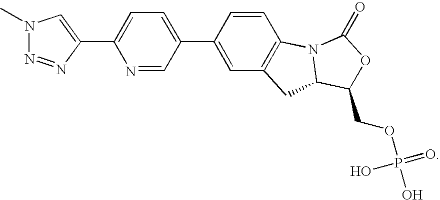

Scheme for Compound of Example 40:

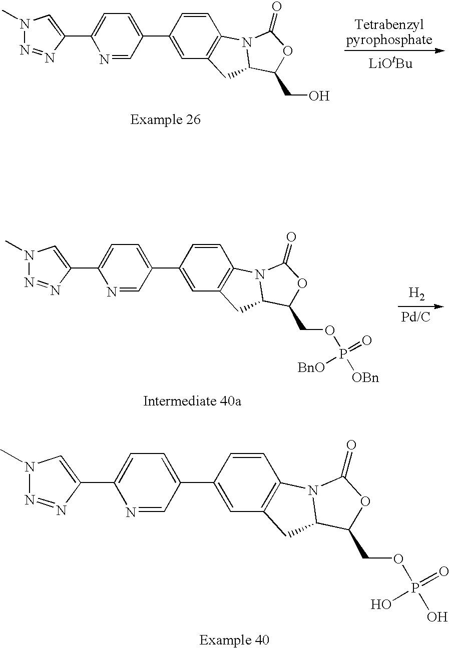

Compound of Example 40. This compound is prepared according to a similar procedure in preparation of Compound of Example 4, except that Compound of Example 1 is substituted for Compound of Example 26.

### Example 41

### Compound of Structure

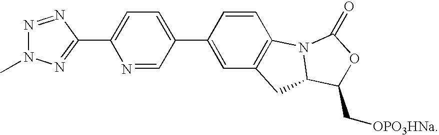

Scheme for Compound of Example 41:

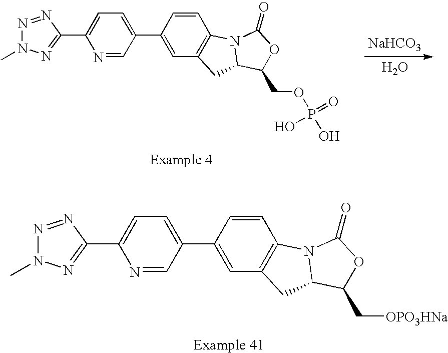

Compound of Example 41. This compound is made according to a similar procedure in the preparation of Compound of Example 5 (Method A), except that Na₂CO₃ is substituted for NaHCO₃, and 1.0 eq. NaHCO₃ is used for 1.0 eq of the Compound of Example 4.

### Example 42

### Compound of Structure

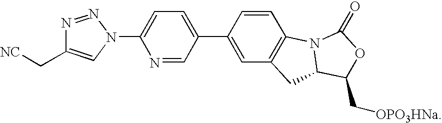

Scheme for Compound of Example 42:

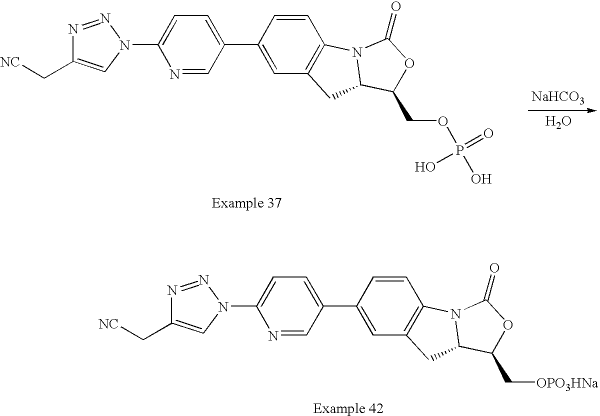

Compound of Example 42. This compound is made according to a similar procedure in the preparation of Compound of Example 5 (Method A), except that Compound of Example 4 is substituted by Compound of Example 37, Na₂CO₃ is substituted for NaHCO₃, and 1.0 eq. NaHCO₃ is used for 1.0 eq of the Compound of Example 37.

### Utility and Testing

Compounds of the subject invention exhibit potent activities against a variety of microorganisms, including gram positive microorganisms. Accordingly, compounds of the subject invention have useful antibacterial activity. Thus, compounds of the present invention are useful antimicrobial agents and may be effective against a number of human and veterinary pathogens, including gram positive aerobic bacteria such as multiply-resistant staphylococci, enterococci, and streptococci, as well as anaerobic microorganisms such as bacteroides and clostridia species, and acid-fast microorganisms such as Mycobacterium tuberculosis and Mycobacterium avium.

Compounds of this invention can have useful activity against a variety of pathogenic microorganisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pennsylvania, USA. Minimum inhibitory concentration (MIC) refers to the lowest concentration of drug (μg/mL) that inhibits visible growth of the organism. Lower MIC values indicate a higher antibacterial activity. Typically, compounds of present invention have useful potency against Gram-positive or Gram-negative pathogens with MIC values of ≦16 μg/mL. The useful activity of compounds of the present invention against a clinical isolate of methicillin-resistant Staphylococcus aureus (MRSA; from the Massachusetts General Hospital, USA) is illustrated by the MIC data of Table 1.

TABLE 1
Antibacterial Activity (MIC) Against MRSA

| | MRSA, |
|---|---|
| | MIC, |
| EXAMPLES | μg/mL |
| Linezolid | 2.0 |
| Example 1 | 0.5 |
| Example 2 | 0.5 |
| Example 3 | 0.25 |
| Example 7 | 1 |
| Example 12 | 1 |
| Example 13 | 1 |
| Example 14 | 1 |
| Example 15 | 4 |
| Example 16 | 0.5 |
| Example 17 | 1 |
| Example 21 | 0.25 |
| Example 23 | 0.25 |
| Example 24 | 0.25 |
| Example 25 | 0.25 |
| Example 21 | 0.25 |
| Example 23 | 0.25 |
| Example 24 | 0.25 |
| Example 25 | 0.25 |
| Example 26 | 1 |
| Example 27 | 0.25 |
| Example 28 | 0.25 |
| Example 29 | 1 |
| Example 30 | 0.25 |

The in vivo activity of compounds of this invention is exemplified by the data summarized in the following Table 2. Determination of in vivo efficacy was performed by inoculating mice intraperitoneally with cultures of infecting organism using Linezolid as control. The compound was dissolved in DMSO with 20% cyclodextrin, diluted with deionized water, and administered orally (p.o.). The mice were dosed 1 hours after infection. Mortality was recorded after 72 hours. The 50% effective dose (ED₅₀, mg/kg) was calculated at the end of the test.

TABLE 2
In vivo Antibacterial Efficacy (ED₅₀) Against S. aureus

| | ED₅₀ |
|---|---|
| EXAMPLES | mg/kg, p.o. |
| Example 1 | 4.2 |
| Example 2 | 6.1 |
| Example 3 | 3.3 |
| Example 5 | 5.6 |
| Example 21 | 9.5 |
| Example 23 | 5.3 |
| Example 24 | 9.8 |
| Example 25 | 9.6 |
| Example 32 | 10.0 |
| Example 33 | 9.1 |
| Example 39 | 10.0 |

Monoamine oxidase inhibitory and myelosuppression (i.e. bone marrow or hematopoietic toxicity) for compounds invented herein can be assessed using established protocols as described below.

Human monoamine oxidase (MOA) A type enzyme inhibition activity for select compounds was measured using a commercial MAO assay kit MAO-Glo™ from Promega Co. (USA). The assay was performed as described in the company's technical bulletin “MAO-Glo™ Assay”. The protocol involves an incubation of the MAO A enzyme (BD Gentest™) with a luminogenic MAO substrate to produce an enzymatic product which is converted to luciferin by a coupled reaction. The released luciferin undergoes further transformation to generate light that is detected and measured. The amount of the light is directly proportional to the activity of MAO. Percent inhibition at several concentrations is established relative to the uninhibited control rate, and the IC₅₀ (μg/mL) values are calculated. A low IC₅₀ value indicates that the tested inhibitor possesses a strong affinity or binding to MAO enzyme, thus being a stronger inhibitor, as compared to the compound with a higher IC₅₀ value. The MAO inhibition data for select compound of this invention are illustrated in the Table 3 below.

As evident from the data of the Table 3, certain compounds of the present invention offer a significantly reduced MAO inhibition over the current antibacterial therapy standard of this class linezolid (Zyvox^{R}).

TABLE 3
Monoamine Oxidase A Inhibition

| | MAO A |
|---|---|
| EXAMPLES | IC₅₀, μg/mL |
| Linezolid | 4.1 |
| Example 7 | >100 |
| Example 13 | >100 |
| Example 14 | >100 |
| Example 16 | 16.8 |

Myelosuppressive potential (hematopoietic or bone marrow toxicity) was evaluated using human CD34⁺ bone marrow cells, generally following methods described by Leach in International Patent Publication No. WO 2006/097828. Thus, an oxazolidinone compound was incubated with fresh human bone marrow cells for 9-10 days at 37° C. in 5% CO₂ atmosphere. At end of the incubation period, the bone marrow toxicity was accessed by measuring inhibition (IC₅₀, μg/mL) of CD34⁺ cell growth using a luminescence assay. Lower IC₅₀ value indicates a higher myelosuppression potential with enhanced probability of undesired adverse effects in vivo, while a higher IC₅₀ value indicates reduced bone marrow toxicity. Results are illustrated by the Table 4 below. For comparison, the data are represented as normalized IC₅₀ ratio indicating the fold of improvement over the reference compound of Example 18, based on IC₅₀ for each compound in the CD34 assay. Thus, the higher ratio in Table 4 indicates a beneficially reduced myelosuppression potential (hematopoietic toxicity) for a compound of the present invention, over the comparator compound of Example 18.

TABLE 4
Bone Marrow CD34⁺ Cells Growth Inhibition (IC₅₀)

| EXAMPLES | CD34 IC₅₀ RATIO |
|---|---|
| Example 1 | 4.6 |
| Example 2 | 3.3 |
| Example 12 | 2.5 |
| Example 13 | 2.5 |
| Example 14 | 2.0 |
| Example 17 | 4.5 |
| Example 18 | 1 |
| Example 20 | 14.8 |
| Example 21 | 2.3 |
| Example 22 | 8.3 |
| Example 23 | 2.5 |
| Example 24 | 1.6 |
| Example 25 | 4.9 |
| Example 26 | 11.4 |
| Example 27 | 1.6 |
| Example 28 | 2.3 |
| Example 29 | 15.5 |
| Example 30 | 4.1 |

As evident from the data of the Table 4, certain compounds of the present invention featuring groups Z (incorporated into the formula I) offer a significantly reduced bone marrow inhibition over analogs lacking the aforementioned structural element, exemplified by the data for the compound of Example 18, wherein Z is H. This beneficial effect can be translated into attenuated mammalian toxicity, including but not limited to sub-acute tolerability and repeated dose toxicity.

Thus, the biological testing data of Tables 2 and 3 illustrate that certain compounds of this invention offer an excellent antibacterial activity and efficacy in vivo beneficially coupled with a significantly reduced propensity for monoamine oxidase inhibition and myelosuppression toxicity.

### Administration and Pharmaceutical Formulations

In general, the compounds of the subject invention can be administered in a therapeutically effective amount by any of the accepted modes of administration for agents that serve similar utilities. By way of example, compounds of the subject invention may be administered orally, parenterally, transdermally, topically, rectally, or intranasally. The actual amount of a compound of the subject invention, i.e., the active ingredient, will depend on a number of factors, such as the severity of the disease, i.e., the infection, to be treated, the age and relative health of the subject, the potency of the compound used, the route and form of administration, and other factors, all of which are within the purview of the attending clinician.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

When employed as pharmaceuticals, the compounds of the subject invention are usually administered in the form of pharmaceutical compositions. These compounds can be administered by a variety of routes including oral, parenteral, transdermal, topical, rectal, and intranasal.

Compounds provided herein are effective as injectable, oral, inhaleable, or topical compositions. Such compositions are prepared in a manner well known in the pharmaceutical art and comprise at least one active compound.

This invention also includes pharmaceutical compositions which contain, as the active ingredient, one or more of the compounds of the subject invention above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The quantity of active component, that is the compound according to the subject invention, in the pharmaceutical composition and unit dosage form thereof may be varied or adjusted widely depending upon the particular application, the potency of the particular compound and the desired concentration.

The compositions are preferably formulated in a unit dosage form, each dosage containing from about 0.1 to about 2000 mg, more usually about 1 to about 900 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of the subject invention above is employed at no more than about 20 weight percent of the pharmaceutical composition, more preferably no more than about 15 weight percent, with the balance being pharmaceutically inert carrier(s).

An active compound is effective over a wide dosage range and is generally administered in a pharmaceutically or therapeutically effective amount. It, will be understood, however, that the amount of the compound actually administered can be determined by a physician, in the light of the relevant circumstances, including the condition to be treated, the severity of the bacterial infection being treated, the chosen route of administration, the actual compound administered, the age, weight, and response of the individual patient, the severity of the patient's symptoms, and the like.

In therapeutic use for treating, or combating, bacterial infections in warm-blooded animals, compounds or pharmaceutical compositions thereof can be administered orally, topically, transdermally, and/or parenterally at a dosage to obtain and maintain a concentration, that is, an amount, or blood-level of active component in the animal undergoing treatment which will be antibacterially effective. Generally, such antibacterially or therapeutically effective amount of dosage of active component (i.e., an effective dosage) will be in the range of about 0.1 mg/kg to about 250 mg/kg, more preferably about 1.0 mg/kg to about 50 mg/kg of body weight/day.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

The following formulation examples illustrate representative pharmaceutical compositions of the present invention. Amount of a compound of present invention in a formulation composition can be in a range of 10-10000 mg. Preferably, said amount can be in a range of 20-900 mg. More preferably, said amount can be in a range of 50-750 mg, or even more preferably, in a range of 200-600 mg.

### Formulation Example 1

Hard gelatin capsules containing the following ingredients are prepared:

| | Quantity |
|---|---|
| Ingredient | (mg/capsule) |
| Active Ingredient | 200-600 |
| Starch | 100-300 |
| Magnesium stearate | 5-15 |

The above ingredients are mixed and filled into hard gelatin capsules for oral administration.

### Formulation Example 2

A tablet formula is prepared using the ingredients below:

| | Quantity |
|---|---|
| Ingredient | (mg/tablet) |
| Active Ingredient | 50-750 |
| Cellulose, microcrystalline | 100-250 |
| Colloidal silicon dioxide | 10-20 |
| Stearic acid | 5-10 |

The components are blended and compressed to form tablets for oral administration.

### Formulation Example 3

A dry powder inhaler formulation is prepared containing the following components:

| Ingredient | Weight % |
|---|---|
| Active Ingredient | 100-600 |
| Lactose | 40-100 |

The active ingredient is mixed with the lactose and the mixture is added to a dry powder inhaling appliance.

### Formulation Example 4

Tablets, each containing 200-600 mg of active ingredient, are prepared as follows

| | Quantity | |
|---|---|---|
| Ingredient | (mg/tablet) | |
| Active Ingredient | 200-600 | mg |
| Starch | 15-45 | mg |
| Microcrystalline cellulose | 10-35 | mg |
| Polyvinylpyrrolidone | 5-10 | mg |
| (as 10% solution in sterile water) | | |
| Sodium carboxymethyl starch | 5-10 | mg |
| Magnesium stearate | 0.5-2 | mg |
| Talc | 1.0-5 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets for oral administration.

### Formulation Example 5

Capsules, each containing 200-600 mg of medicament are made as follows:

| | Quantity |
|---|---|
| Ingredient | (mg/capsule) |
| Active Ingredient | 200-600 mg |
| Starch | 75-150 mg |
| Magnesium stearate | 1-4 mg |

The active ingredient, starch and magnesium stearate are blended, passed through a No. 20 mesh U.S. sieve, and filled into hard gelatin capsules for oral administration.

### Formulation Example 6

Suppositories, each containing 200-600 mg of active ingredient are made as follows:

| Ingredient | Amount |
|---|---|
| Active Ingredient | 200-600 mg |
| Saturated fatty acid glycerides to | 1000-2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 200-600 mg of medicament per 7 mL dose are made as follows:

| Ingredient | Amount | |
|---|---|---|
| Active Ingredient | 200-600 | mg |
| Xanthan gum | 2-8 | mg |
| Sodium carboxymethyl cellulose (11%) | | |
| Microcrystalline cellulose (89%) | 20-50 | mg |
| Sucrose | 1.0-1.75 | g |
| Sodium benzoate | 10-20 | mg |
| Flavor and Color | q.v. | |
| Purified water to | 5-7 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

| | Quantity |
|---|---|
| Ingredient | (mg/capsule) |
| Active Ingredient | 200-600 mg |
| Starch | 200-410 mg |
| Magnesium stearate | 3-6 mg |

The active ingredient, starch, and magnesium stearate are blended, passed through a No. 20 mesh U.S. sieve, and filled into hard gelatin capsules for oral administration.

### Formulation Example 9

A subcutaneous formulation may be prepared as follows:

| Ingredient | Quantity | |
|---|---|---|
| Active Ingredient | 200-600 | mg |
| Corn Oil | 1.0-1.5 | mL |

### Formulation Example 10

A topical formulation may be prepared as follows:

| Ingredient | Quantity | |
|---|---|---|
| Active Ingredient | 1-10 | g |
| Emulsifying Wax | 20-30 | g |
| Liquid Paraffin | 10-20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions that can transiently open the blood-brain barrier.

Other suitable formulations for use in the present invention can be found in Remington's Pharmaceutical Sciences, Mace Publishing Company, Philadelphia, Pa., 17th ed. (1985).

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference.

As noted above, the compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

The disclosures of each and every patent, patent application and publication (for example, journals, articles and/or textbooks) cited herein are hereby incorporated by reference in their entirety. Also, as used herein and in the appended claims, singular articles such as “a”, “an” and “one” are intended to refer to singular or plural. While the present invention has been described herein in conjunction with a preferred aspect, a person with ordinary skills in the art, after reading the foregoing specification, can affect changes, substitutions of equivalents and other types of alterations to the invention as set forth herein. Each aspect described above can also have included or incorporated therewith such variations or aspects as disclosed in regard to any or all of the other aspects. The present invention is also not to be limited in terms of the particular aspects described herein, which are intended as single illustrations of individual aspects of the invention. Many modifications and variations of this invention can be made without departing from its spirit and scope, as will be apparent to those skilled in the art. Functionally equivalent methods within the scope of this invention, in addition to those enumerated herein, will be apparent to those skilled in the art from the foregoing descriptions. It is to be understood that this invention is not limited to particular methods, reagents, process conditions, materials and so forth, which can, of course, vary. It is also to be understood that the terminology used herein is for the purpose of describing particular aspects only, and is not intended to be limiting. Thus, it is intended that the specification be considered as exemplary.

What is claimed is:

1. A compound according to formula I 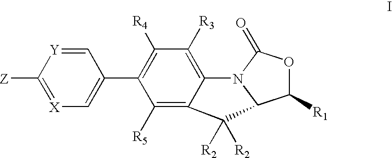
or a pharmaceutically acceptable salt, solvate, or hydrate thereof, wherein:
R¹ is CH₂OH, CH₂NHC(═O)C₁₋₅alkyl, CH₂NHC(═O)OC₁₋₅alkyl, CH₂NH-Het¹, CH₂O-Het¹CH₂Het¹, CH₂Het², CH₂OPO₃H₂, CH₂OC(═O)CH₂(CH₂)ₘOPO₃H₂, or CH₂OC(═O)CH(NH₂)C₁₋₄alkyl, wherein m is 1, 2, or 3;
R² is H or F;
R³, R⁴, and R⁵ are each independently H, F, Cl, CN, CH₃, or OH;
X and Y are each independently CH, CF, N, or N⁺—O⁻;
Z is Het¹Het², 4 to 7-membered heterocyclic group, CN, CONH₂, CONHC₁₋₆alkyl, NH—C(═O)H, NH—C(═O)C₁₋₆alkyl, NH—SO₂C₁₋₆alkyl, NH—C(═O)OC₁₋₆alkyl, NHC(═O)NHC₁₋₆alkyl, 4-(Het¹-CH₂—W—CH₂)— or 4-(Het²-CH₂—W—CH₂)—, wherein W is CH₂, NH, NC₁₋₅alkyl, NC(═O)OC₁₋₅alkyl, NC(═O)C₁₋₅alkyl, NC(═O)Het¹, O, S, S(O), and n is 0, 1, or 2;
each Het¹ is independently a carbon-connected tetrazole, 1,2,3-triazole, 1,2,4-triazole, 1,3,4-oxadiazole, 1,3,4-thiadiazole, 1,2,4-oxadiazole, oxazole, thiazole, isoxazole, isothiazole, isoxazoline, or pyrazole group; and
each Het² is independently a nitrogen-connected tetrazole, 1,2,3-triazole, 1,2,4-triazole, oxazolidinone, pyrrolidin-2-one, imidazolidin-2-one, pyrazole, or imidazole group.

2. The compound of claim 1, with a proviso that, when R¹ is CH₂NHCOR′, wherein R′ is selected from
H;
C₁₋₁₂alkyl,
C₁₋₁₂alkyl optionally substituted with 1-3 Cl;
CH₂OH;
CH₂OC₁₋₁₂alkyl;
C₃₋₁₂cycloalkyl;
phenyl optionally substituted with 1-3 of groups OH, OMe, OEt, NO₂, halo, COOH, SO₃H, or NR″R′″, wherein R″ and R′″ are selected from H and C₁₋₁₂alkyl;
furanyl;
tetrahydrofuranyl;
2-thiophene;
pyrrolidinyl;
pyridinyl;
OC₁₋₁₂alkyl;
NH₂;
NHC₁₋₁₂alkyl;
NHPh;
COPh; and
X is CH or CF, and Y is N or N⁺—O⁻; then
Z is other than H, C₁₋₄alkyl, NO₂, NH₂, CN, COOH, OC₁₋₄alkyl, or halo.

3. The compound of claim 1, wherein X is CH or CF, and Y is N or N⁺—O⁻, with a proviso that
when R¹ is CH₂NHCOR′, wherein R′ is selected from
H;
C₁₋₁₂alkyl,
C₁₋₁₂alkyl optionally substituted with 1-3 Cl;
CH₂OH;
CH₂OC₁₋₁₂alkyl;
C₃₋₁₂cycloalkyl;
phenyl optionally substituted with 1-3 of groups OH, OMe, OEt, NO₂, halo, COOH, SO₃H, or NR″R′″, wherein R″ and R′″ are selected from H and C₁₋₁₂alkyl;
furanyl;
tetrahydrofuranyl;
2-thiophene;
pyrrolidinyl;
pyridinyl;
OC₁₋₁₂alkyl;
NH₂;
NHC₁₋₁₂alkyl;
NHPh;
COPh; then
Z is other than H, NO₂, NH₂, NHC(═O)C₁₋₄alkyl, CN, COOH, OC₁₋₄alkyl, or halo.

4. The compound of claim 1, wherein Z is Het¹ or Het².

5. The compound of claim 1, wherein R¹ is CH₂OH.

6. The compound of claim 1, wherein R² is H; and R³, R⁴ and R⁵ are each independently selected from H and F.

7. The compound of claim 1 selected from:

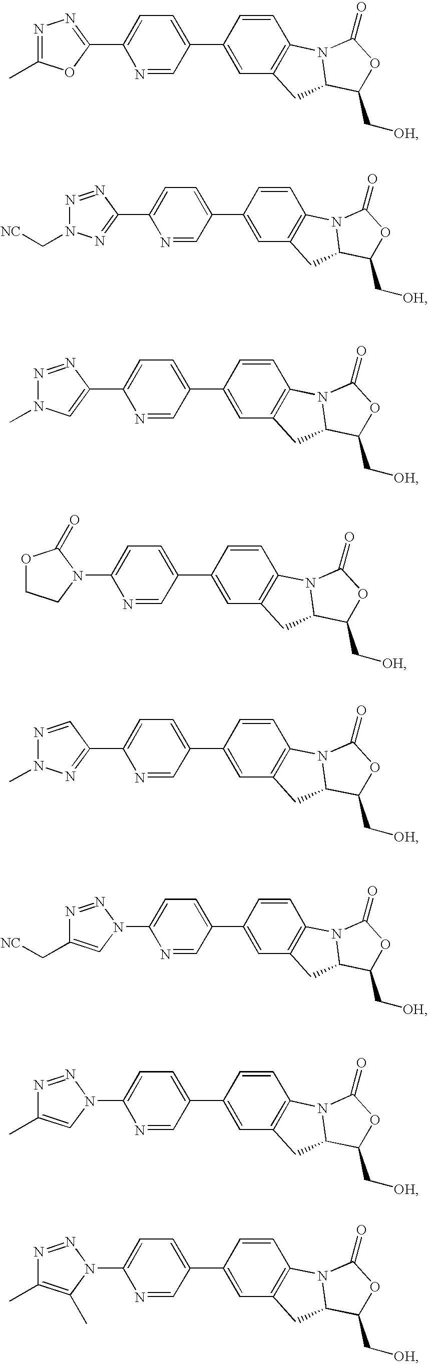

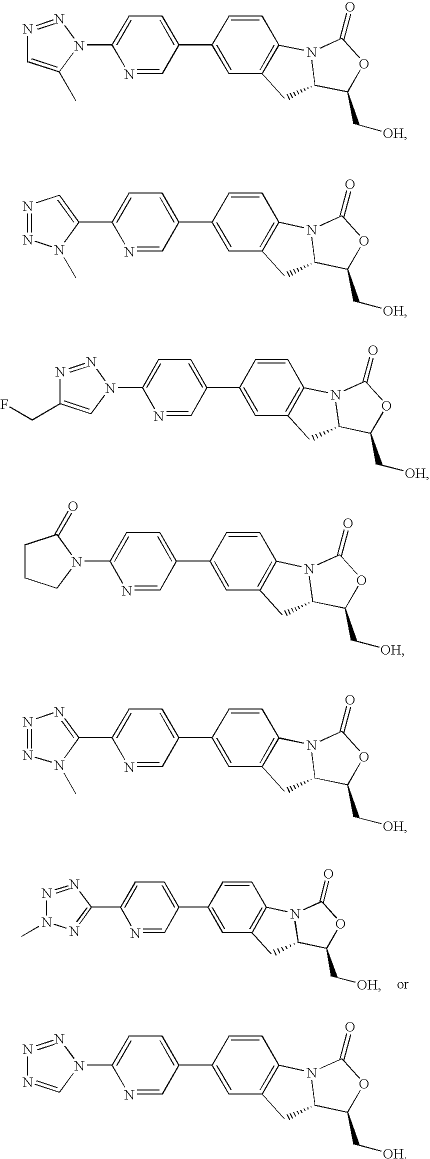

8. The compound of claim 1 selected from:

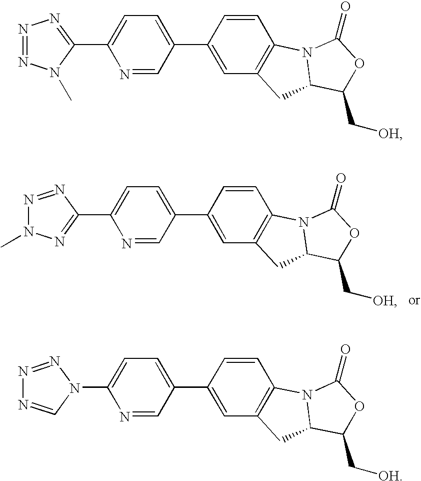

9. The compound of claim 1 selected from:

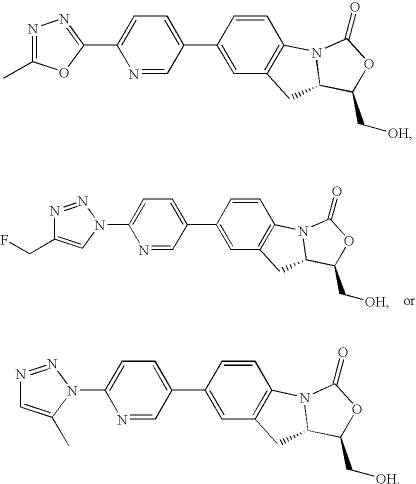

10. The compound of claim 1 selected from:

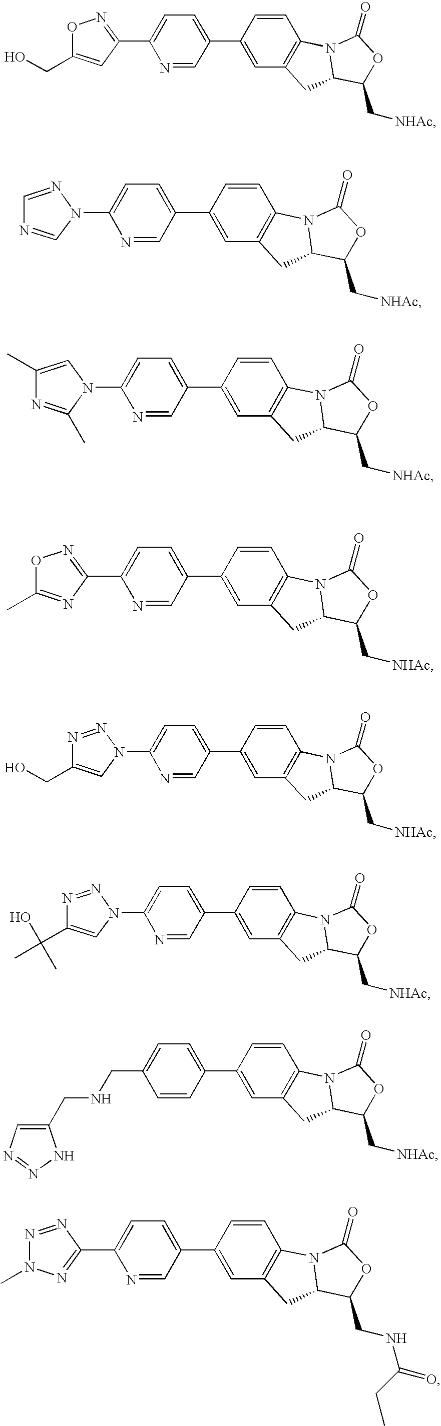

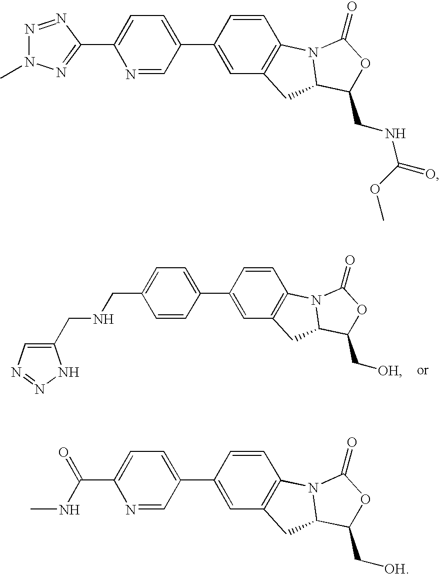

11. The compound of claim 1 selected from:

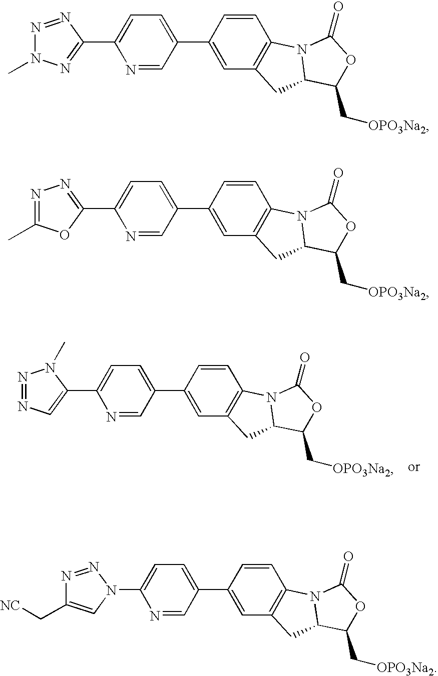

12. The compound of claim 1 selected from:

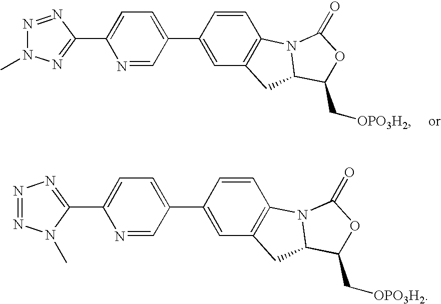

13. The compound of claim 1 selected from:

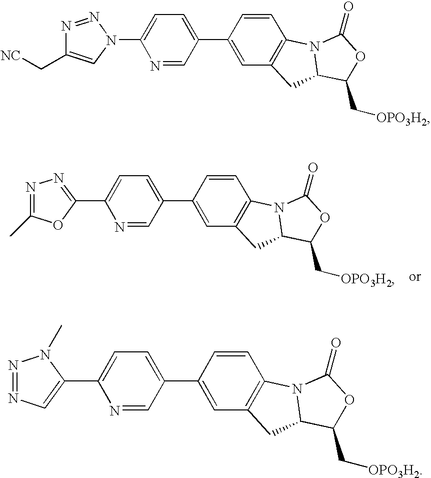

14. The compound of claim 1 selected from:

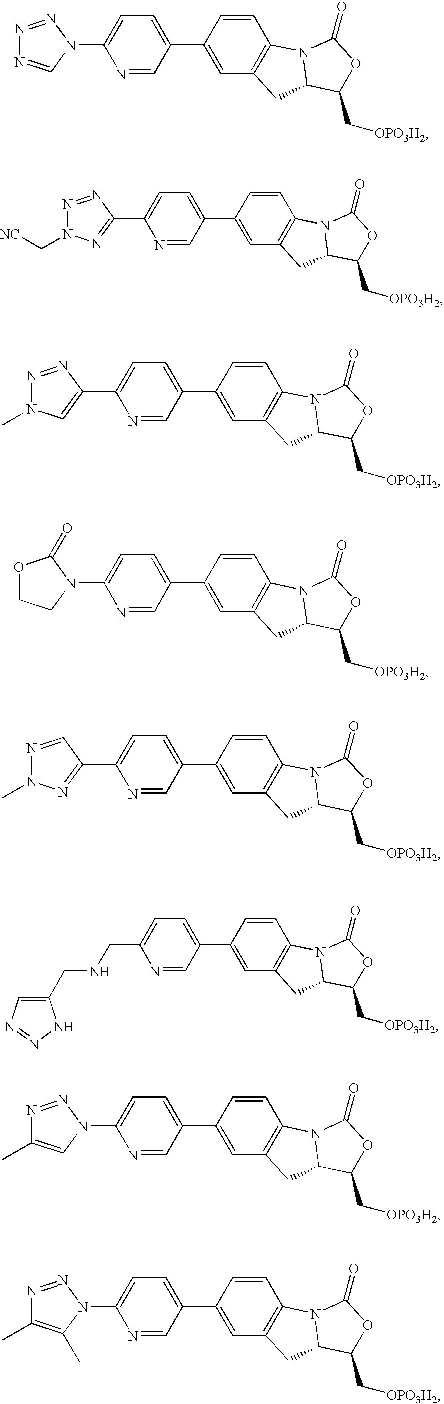

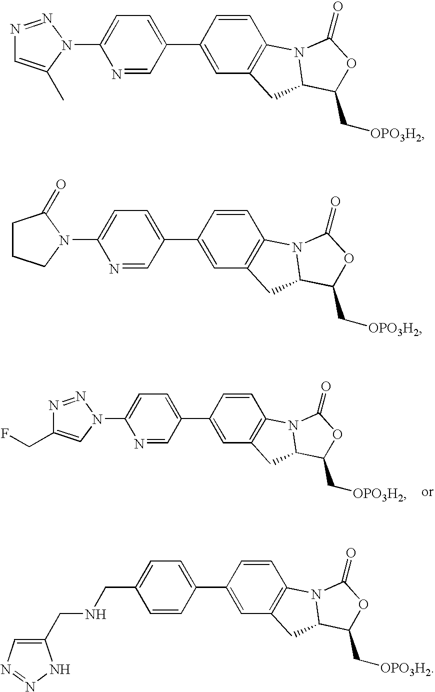

15. The compound of claim 1 selected from:

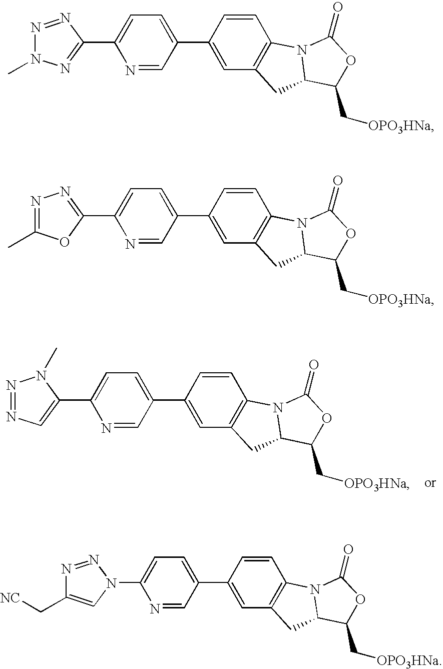

16. The compound of claim 1 selected from:

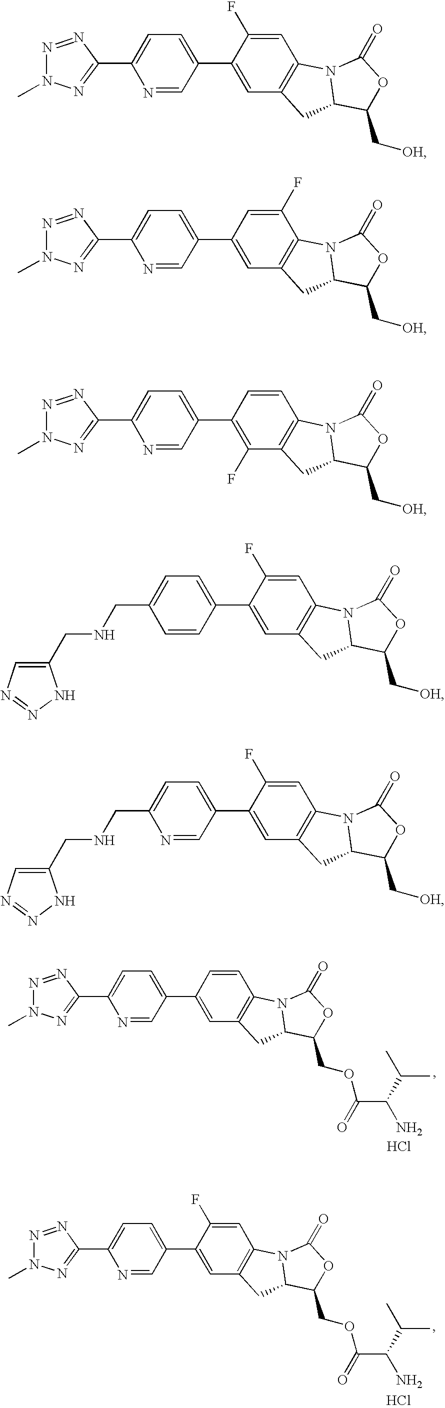

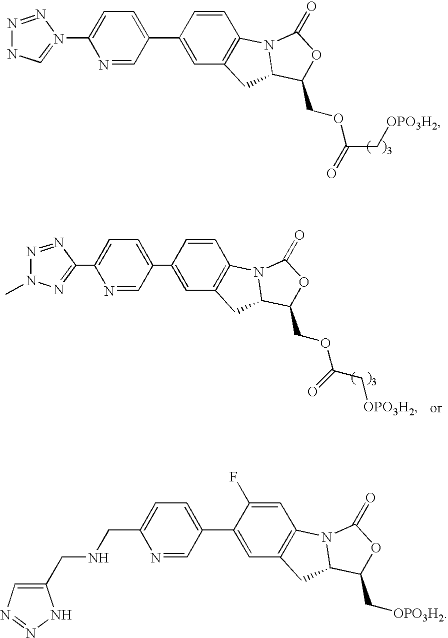

17. A method for the treatment of a microbial infection in a mammal comprising administering to the mammal a therapeutically effective amount of a compound of claim 1.

18. The method according to claim 17, wherein the compound is administered to the mammal orally, parenterally, transdermally, topically, rectally, or intranasally in a pharmaceutical composition.

19. The method according to claim 17, wherein the compound is administered once-daily in an amount of from about 1 to about 75 mg/kg of body weight/day.

20. A pharmaceutical composition comprising a therapeutically effective amount of a compound of claim 1 and a pharmaceutically acceptable carrier.
